SEQUENCE LISTING (2 sequences)

SEQ ID NO: 1 - DNA, 1554 bp, Bacillus amyloliquefaciens
ctttatcggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagkacggtcgcaagactgaaactcaaaggaattgacgggggcccgcacaagcggkggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctcctttct

SEQ ID NO: 2 - DNA, 3246 bp, Bacillus amyloliquefaciens
atgggtgatttccctattatgacagataccggtacttttatcatcaacggtgcagaacgtgttatcgtatctcagcttgttcggtctccaagtgtatatttcagtggtaaagtagacaagaacggtaaaaaaggttttaccgcgactgtcattccaaaccgtggcgcatggttagaatacgaaactgatgcgaaagatgttgtgtatgtccgcattgatcgcacacgtaagttgccggttacggttcttttgcgtgctctcggcttcggttccgaccaagagattctcgatctcattggtgagaacgaatatctccgcaatacactggataaggacaacactgaaaacagtgacaaagcgcttcttgaaatctatgagcgccttcgtcccggagagccgcctacagtagaaaacgcaaaaagcttgctggattcccgtttcttcgatccgaagcgatacgaccttgcgaatgtaggacgctataaaattaataaaaagcttcatatcaagaaccgcctgtttaaccagcgccttgcagaaacactggtggatccggaaaccggtgaaattctcgctgaaaaagggcagattcttgacagaagaacacttgataaagtactgccatacttagaaaatggaatcggcttcagaaagctttatcctaacggcggcgttgtcgaggatgaagtgatgcttcaatccattaaaatctatgctcctaccgatgcagaaggagagcagacgatcaatgtgatcggcaatgcttacatcgaagaggcgattaaaaacattacgcctgctgatattatttcttctatcagctacttcttcaacctcctgcacggagtgggcgacactgatgatatcgaccatctcggaaaccgccgtctgcgttctgtaggtgagctcctgcaaaaccaattccgtatcggtttaagccggatggaacgtgtcgtacgtgaaagaatgtctattcaagacacgaatacaattacgccgcagcagctgattaacatcagacctgttattgcgtctattaaagagttcttcggaagctcacagctttctcaattcatggatcagacgaacccgcttgctgaattgacgcacaaacgccgtctgtcagctctcggaccgggcggtttgacacgtgagcgcgcaggtatggaagtacgtgacgttcactactctcactatggccgtatgtgtccgattgaaacgcctgagggcccgaacatcggtttgatcaactcattgtcatcatttgcgaaagtaaaccgctttggtttcattgagacgccataccgccgcgttgatcctgaaacaggaaaagtaacgcctagaatcgactacctgactgctgatgaagaggataactatgtcgtagcccaagcgaatgctaagctgagcgatgacggttctttcttggatgacagcatcgtagcgcgtttcagaggggaaaacaccgttgtagcccgcaaccgcgtggattacatggacgtatctcctaaacaggttgtatctgctgcgacagcatgtattccgttcttggaaaacgatgactcgaaccgcgccctcatgggagcgaacatgcagcgtcaggctgtgcctttgatgcagccggaagctccgatcgtcggaacgggtatggaatacgtatccggtaaagactccggtgcagccgttatttgtaaacaccctggtatcgttgaacgggtggaagcgaaaaacgtatgggtgcgccgctatgaagaaattgacggccaaaaagtaaaaggcaacctggataagtacagcttgctgaaatttgtccgctccaaccaaggtacgtgctacaaccagcgtccgatcgtcagtgtcggcgatgaagtagtcaaaggagaaatccttgctgacggaccttcaatggagcttggtgaacttgctctcggccgcaacgtaatggtcggcttcatgacatgggatggttacaactatgaggatgccatcatcatgagtgaacgccttgtgaaagatgatgtatacacatctattcacattgaagaatatgaatcagaagcacgtgatacaaagcttgggccggaagagatcacccgcgatattccaaacgtaggggaagacgcgcttcgcaatcttgatgaccgcggaattatccgtatcggtgcggaagtcaacgacggagaccttctcgtaggtaaagtaacgcctaaaggtgtaactgagcttacggctgaagaacgccttctgcatgcgatctttggagaaaaagcgcgtgaagtccgtgatacttctctccgtgtgcctcacggcggcggcggaattatccacgacgtaaaagtcttcaaccgtgaagacggcgacgaacttcctccgggagtgaaccagcttgtacgcgtatatatcgttcagaaacgtaagatttctgaaggtgataaaatggccggacgtcacggaaacaaaggggttatctcgaagattcttcctgaagaagatatgccttaccttcctgacggcacgccgatcgatatcatgcttaacccgctgggtgtaccatcacgtatgaatatcggtcaggtattagaacttcacatgggtatggctgcccgctacctcggcattcacatcgcgtcacctgtatttgacggcgcgcgtgaagaagatgtgtgggaaacacttgaagaagcaggcatgtcaagagacgctaaaacagttctttatgacggccgtacgggtgaaccgtttgacaaccgtgtatctgtcggaatcatgtacatgatcaaactggcacacatggttgatgataaacttcatgcccgttctacaggtccttactcacttgttacgcagcagcctctcggcggtaaagcccaattcggcggacagcgtttcggtgagatggaggtttgggcgcttgaagcttacggcgcagcttacacgcttcaagaaatcctgactgtgaagtccgatgacgtggtcggacgtgtgaaaacatatgaagccatcgtcaaaggcgacaatgttccagagcctggtgttccggaatcattcaaagtattgatcaaagagcttcaaagcttaggtatggacgtgaaaatcctttcaggcgatgaagaagaaatagaaatgagagatctagaagacgaggaagatgcgaaacaagctgacggccttgcattatcaggtgatgaagcgccggaagaaacagcatctccagacgttgaacgtgacgcagtaacgaaagaatag

(19) United States
(12) Patent Application Publication
(10) Pub. No.: US 2016/0186273 A1
(43) Pub. Date: Jun. 30, 2016

(54) BACILLUS AMYLOLIQUEFACIENS RTI301 COMPOSITIONS AND METHODS OF USE FOR BENEFITING PLANT GROWTH AND TREATING PLANT DISEASE

(71) Applicant: FMC Corporation, Philadelphia, PA (US)

(72) Inventors: Safiyh Taghavi, Chapel Hill, NC (US); Daniel van der Lelie, Chapel Hill, NC (US)

(21) Appl. No.: 14/980,123

(22) Filed: Dec. 28, 2015

Related U.S. Application Data
(60) Provisional application No. 62/097,203, filed on Dec. 29, 2014.

(51) Int. Cl. C12R 1/07; A01N 63/02; A01N 25/00; C12N 1/20

(52) U.S. Cl. CPC C12R 1/07; C12N 1/20; A01N 63/02; A01N 25/00

(57) ABSTRACT

Compositions and methods include a new strain of Bacillus amyloliquefaciens having growth promoting activity and activity against plant pathogens. The compositions are useful for benefiting plant growth and/or conferring protection against a pathogenic infection when applied to plant foliage, flowers, fruits, bark, roots, seeds, callus tissue, grafts, cuttings, surrounding soil or growth medium, and soil or growth medium concomitant with sowing seed and planting callus tissue, grafts, and cuttings. The compositions containing the Bacillus amyloliquefaciens RTI301 strain can be applied alone or in combination with other microbial, biological, or chemical insecticides, fungicides, nematicides, bacteriocides, herbicides, plant extracts, plant growth regulators, and fertilizers. In one example, the Bacillus amyloliquefaciens RTI301 strain can be delivered to the plant as part of an integrated pest management program, with other microbial or chemical insecticides, fungicides, nematicides, bacteriocides, herbicides, plant extracts, and plant growth regulators.

### CROSS REFERENCE TO RELATED APPLICATIONS

This application claims the benefit of U.S. provisional application number 62/097,203 filed Dec. 29, 2014, the disclosure of which is hereby incorporated herein by reference in its entirety.

### TECHNICAL FIELD

The presently disclosed subject matter relates to compositions comprising an isolated strain of Bacillus amyloliquefaciens RTI301 for application to plant foliage, plant fruits and flowers, plant seeds and roots, and the soil surrounding plants to benefit plant growth and to treat plant plant disease(s).

### BACKGROUND

A number of microorganisms having beneficial effects on plant growth and health are known to be present in the soil, to live in association with plants specifically in the root zone (Plant Growth Promoting Rhizobacteria “PGPR”), or to reside as endophytes within the plant. Their beneficial plant growth promoting properties include nitrogen fixation, iron chelation, phosphate solubilization, inhibition of non-beneficial microrganisms, resistance to pests, Induced Systemic Resistance (ISR), Systemic Acquired Resistance (SAR), decomposition of plant material in soil to increase useful soil organic matter, and synthesis of phytohormones such as indole-acetic acid (IAA), acetoin and 2,3-butanediol that stimulate plant growth, development and responses to environmental stresses such as drought. In addition, these microorganisms can interfere with a plant's ethylene stress response by breaking down the precursor molecule, 1-aminocyclopropane-1-carboxylate (ACC), thereby stimulating plant growth and slowing fruit ripening. These beneficial microorganisms can improve soil quality, plant growth, yield, and quality of crops. Various microorganisms exhibit biological activity such as to be useful to control plant diseases. Such biopesticides (living organisms and the compounds naturally produced by these organisms) are safer and more biodegradable than synthetic fertilizers and pesticides.

Fungal phytopathogens, including but not limited to Botrytis spp. (e.g. Botrytis cinerea), Fusarium spp. (e.g. F. oxysporum and F. graminearum), Rhizoctonia spp. (e.g. R. solani), Magnaporthe spp., Mycosphaerella spp., Puccinia spp. (e.g. P. recondita), Phytopthora spp. and Phakopsora spp. (e.g. P. pachyrhizi), are one type of plant pest that can cause servere economic losses in the agricultural and horticultural industries. Chemical agents can be used to control fungal phytopathogens, but the use of chemical agents suffers from disadvantages including high cost, lack of efficacy, emergence of resistant strains of the fungi, and undesirable environmental impacts. In addition, such chemical treatments tend to be indiscriminant and may adversely affect beneficial bacteria, fungi, and arthropods in addition to the plant pathogen at which the treatments are targeted. A second type of plant pest are bacterial pathogens, including but not limited to Erwinia spp. (such as Erwinia chrysanthemi), Pantoea spp. (such as P. citrea), Xanthomonas (e.g. Xanthomonas campestris), Pseudomonas spp. (such as P. syringae) and Ralstonia spp. (such as R. soleacearum) that cause servere economic losses in the agricultural and horticultural industries. Similar to pathogenic fungi, the use of chemical agents to treat these bacterial pathogens suffers from disadvantages. Viruses and virus-like organisms comprise a third type of plant disease-causing agent that is hard to control, but to which bacterial microorganisms can provide resistance in plants via induced systemic resistance (ISR). Thus, microorganisms that can be applied as biofertilizer and/or biopesticide to control pathogenic fungi, viruses, and bacteria are desirable and in high demand to improve agricultural sustainability. A final type of plant pathogen includes plant pathogenic nematodes and insects, which can cause severe damage and loss of plants.

Some members of the species Bacillus have been reported as biocontrol strains, and some have been applied in commercial products (Joseph W. Kloepper, et al. 2004, Phytopathology Vol. 94, No. 11, 1259-1266). For example, strains currently being used in commercial biocontrol products include: Bacillus pumilus strain QST2808, used as active ingredient in SONATA and BALLAD-PLUS, produced by BAYER CROP SCIENCE; Bacillus pumilus strain GB34, used as active ingredient in YIELDSHIELD, produced by BAYER CROP SCIENCE; Bacillus subtilis strain QST713, used as the active ingredient of SERENADE, produced by BAYER CROP SCIENCE; Bacillus subtilis strain GBO3, used as the active ingredient in KODIAK and SYSTEM3, produced by HELENA CHEMICAL COMPANY. Various strains of Bacillus thuringiensis and Bacillus firmus have been applied as biocontrol agents against nematodes and vector insects and these strains serve as the basis of numerous commercially available biocontrol products, including NORTICA and PONCHO-VOTIVO, produced by BAYER CROP SCIENCE. In addition, Bacillus strains currently being used in commercial biostimulant products include: Bacillus amyloliquefaciens strain FZB42 used as the active ingredient in RHIZOVITAL 42, produced by ABiTEP GmbH, as well as various other Bacillus subtilus species that are included as whole cells including their fermentation extract in biostimulant products, such as FULZYME produced by JHBiotech Inc.

The presently disclosed subject matter provides microbial compositions and methods for their use in benefiting plant growth and disease prevention and control.

### SUMMARY OF THE INVENTION

In one embodiment, a composition is provided comprising a biologically pure culture of Bacillus amyloliquefaciens RTI301 deposited as ATCC No. PTA-121165, or a mutant thereof having all the identifying characteristics thereof, for application to a plant for one or both of benefiting plant growth or conferring protection against a pathogenic infection in a susceptible plant.

In one embodiment, a plant seed is provided coated with a composition comprising spores of a biologically pure culture of Bacillus amyloliquefaciens RTI301 deposited as ATCC No. PTA-121165, or a mutant thereof having all the identifying characteristics thereof, present in an amount suitable to benefit plant growth and/or to confer protection against a pathogenic infection in a susceptible plant.

In one embodiment, a composition is provided for one or both of benefiting plant growth or conferring protection against pathogenic infection in a susceptible plant, the composition comprising a biologically pure culture of Bacillus amyloliquefaciens RTI301 deposited as ATCC No. PTA-121165, or a mutant thereof having all the identifying characteristics thereof, in an amount suitable to benefit plant growth and/or to confer protection against pathogenic infection in the susceptible plant; and one or a combination of a microbial, biological or a chemical insecticide, fungicide, nematicide, bacteriocide, herbicide, plant extract, plant growth regulator, or fertilizer, in an amount suitable to benefit plant growth and/or to confer protection against pathogenic infection in the susceptible plant.

In one embodiment, a method is provided for one or both of benefiting growth of a plant or conferring protection against pathogenic infection in a susceptible plant, the method comprising delivering a composition comprising a biologically pure culture of Bacillus amyloliquefaciens RTI301 deposited as ATCC No. PTA-121165, or a mutant thereof having all the identifying characteristics thereof to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium, in an amount suitable to benefit the plant growth and/or to confer protection against pathogenic infection in the susceptible plant.

In one embodiment, a method is provided for one or both of benefiting growth of a plant or conferring protection against pathogenic infection in a susceptible plant, the method comprising delivering a composition comprising a biologically pure culture of Bacillus amyloliquefaciens RTI301 deposited as ATCC No. PTA-121165, or a mutant thereof having all the identifying characteristics thereof, in an amount suitable for benefiting the plant growth and/or conferring protection against the pathogenic infection; and one or a combination of a microbial, a biological, or a chemical insecticide, fungicide, nematicide, bacteriocide, herbicide, plant extract, plant growth regulator, or fertilizer in an amount suitable for benefiting the plant growth and/or conferring protection against the pathogenic infection, to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In one embodiment, a method is provided for one or both of benefiting growth of a plant or conferring protection against pathogenic infection in a susceptible plant, the method comprising delivering a combination of a first composition comprising a biologically pure culture of Bacillus amyloliquefaciens RTI301 deposited as ATCC No. PTA-121165, or a mutant thereof having all the identifying characteristics thereof in an amount suitable to benefit the plant growth and/or to confer protection against pathogenic infection in the susceptible plant; and a second composition comprising one or a combination of a microbial, a biological, or a chemical insecticide, fungicide, nematicide, bacteriocide, herbicide, plant extract, plant growth regulator, or fertilizer, in an amount suitable to benefit the plant growth and/or to confer protection against pathogenic infection in the susceptible plant, to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In one embodiment, a method is provided for one or both of benefiting growth of a plant or conferring protection against pathogenic infection in a susceptible plant, the method comprising: planting a seed of the plant or regenerating a vegetative cutting/tissue of the plant in a suitable growth medium, wherein the seed has been coated or the vegetative cutting/tissue has been inoculated with a composition comprising a biologically pure culture of Bacillus amyloliquefaciens RTI301 deposited as ATCC PTA-121165, or a mutant thereof having all the identifying characteristics thereof, wherein growth of the plant from the seed or the vegetative cutting/tissue is benefited and/or protection against pathogenic infection is conferred.

In one embodiment, a method is provided for benefiting plant growth by conferring protection against or reducing pathogenic infection in a susceptible plant while minimizing the build-up of resistance against the treatment, the method comprising delivering to the susceptible plant in separate applications and in altering time intervals a first composition and a second composition, wherein each of the first and second compositions are delivered in an amount suitable to to confer protection against or reduce pathogenic infection in the plant, wherein the first composition comprises a biologically pure culture of Bacillus amyloliquefaciens RTI301 deposited as ATCC No. PTA-121165, or a mutant thereof having all the identifying characteristics thereof, and wherein the second composition comprises one or more chemical active agents having fungicidal or a bacteriocidal properties, and wherein the first and second compositions are delivered in the altering time intervals to one or a combination of foliage of the plant, bark of the plant, fruit of the plant, flowers of the plant, seed of the plant, roots of the plant, a cutting of the plant, a graft of the plant, callus tissue of the plant, or soil or growth medium surrounding the plant, wherein the total amount of the chemical active agent(s) required to confer protection against and/or reduce the pathogenic infection is decreased and the build-up of resistance against the treatment is minimized.

In one embodiment, a composition is provided, the composition including at least one of an isolated Fengycin MA compound, an isolated Fengycin MB compound, an isolated Fengycin MC compound, an isolated Dehydroxyfengycin MA compound, an isolated Dehydroxyfengycin MB compound, an isolated Dehydroxyfengycin MC compound, an isolated Fengycin H compound, an isolated Dehyroxyfengycin H compound, an isolated Fengycin I compound, and an isolated Dehyroxyfengycin I compound in an amount suitable to confer one or both of a growth benefit on the plant or protection against a pathogenic infection in a susceptible plant, the Fengycin and Dehyroxyfengycin compounds having the formula:

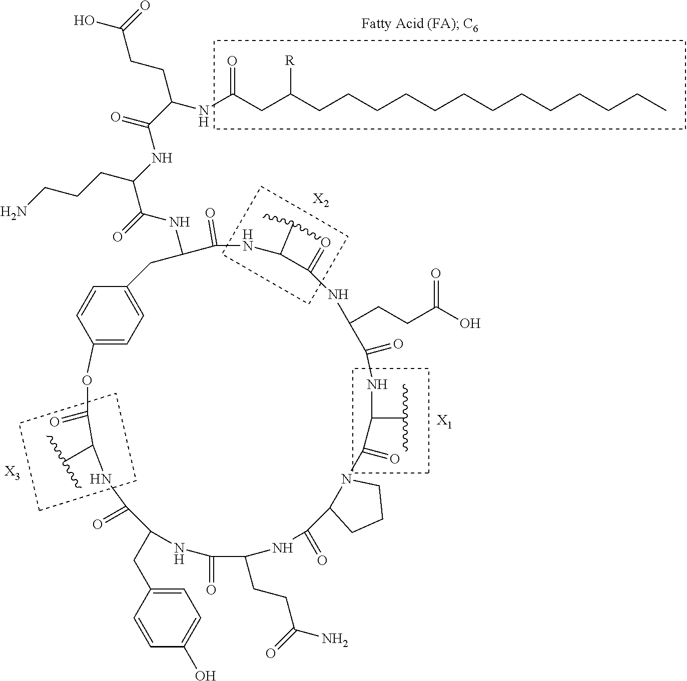

wherein R is OH, n ranges from 8 to 20, FA is linear, iso, or anteiso and: X₁ is Ala, X₂ is Thr, and X₃ is Met for Fengycin MA; X₁ is Val, X₂ is Thr, and X₃ is Met for Fengycin MB; X₁ is Aba, X₂ is Thr, and X₃ is Met for Fengycin MC; X₁ is Val, X₂ is Thr, and X₃ is Hcy for Fengycin H; and X₁ is Ile, X₂ is Thr, and X₃ is Ile for Fengycin I; or wherein R is H, n ranges from 8 to 20, FA is linear, iso, or anteiso and: X₁ is Ala, X₂ is Thr, and X₃ is Met for Dehydroxyfengycin MA; X₁ is Val, X₂ is Thr, and X₃ is Met for Dehydroxyfengycin MB; X₁ is Aba, X₂ is Thr, and X₃ is Met for Dehydroxyfengycin MC; X₁ is Val, X₂ is Thr, and X₃ is Hcy for Dehydroxyfengycin H; and X₁ is Ile, X₂ is Thr, and X₃ is Ile for Dehydroxyfengycin I.

In one embodiment, an extract is provided of a biologically pure culture of a Bacillus amyloliquefaciens strain, the extract including a Fengycin-MA, -MB, -MC, -H, and -I compound and a Dehydroxyfengycin-MA, -MB, -MC, -H, and -I compound and one or a combination of additional Fengycin-and Dehydroxyfengycin-like compounds listed in Table XIII.

In one embodiment, an extract is provided of a biologically pure culture of Bacillus amyloliquefaciens RTI301 deposited as ATCC No. PTA-121165, the extract including a Fengycin-MA, -MB, -MC, -H, and -I compound and a Dehydroxyfengycin-MA, -MB, -MC, -H, and -I compound and one or a combination of additional Fengycin-and Dehydroxyfengycin-like compounds listed in Table XIII.

In one embodiment, a composition is provided for benefiting plant growth, the composition comprising: a biologically pure culture of Bacillus amyloliquefaciens RTI301 deposited as ATCC No. PTA-121165, or a mutant thereof having all the identifying characteristics thereof; and a bifenthrin insecticide.

In one embodiment, a composition is provided for benefiting plant growth, the composition comprising: a biologically pure culture of Bacillus amyloliquefaciens RTI301 deposited as ATCC No. PTA-121165, or a mutant thereof having all the identifying characteristics thereof; and a fungicide comprising one or a combination of an extract from Lupinus albus, a BLAD polypeptide, or a fragment of a BLAD polypeptide.

In one embodiment, a product is provided comprising: a first composition comprising a biologically pure culture of Bacillus amyloliquefaciens RTI301 deposited as ATCC No. PTA-121165, or a mutant thereof having all the identifying characteristics thereof; a second composition comprising one or a combination of a microbial, a biological, or a chemical insecticide, fungicide, nematicide, bacteriocide, herbicide, plant extract, plant growth regulator, or fertilizer, wherein the first and second compositions are separately packaged, and wherein each composition is in an amount suitable for one or both of benefiting plant growth or conferring protection against a pathogenic infection in a susceptible plant; and optionally instructions for delivering in an amount suitable to benefit plant growth, a combination of the first and second compositions to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

### BRIEF DESCRIPTION OF THE FIGURES

FIG. 1 shows a schematic diagram of the genomic organization surrounding and including the unique lantibiotic biosynthesis operon found in Bacillus amyloliquefaciens strain RTI301 as compared to the corresponding regions for two Bacillus amyloliquefaciens reference strains, Bacillus amyloliquefaciens FZB42 and Bacillus amyloliquefaciens TrigoCor1448, according to one or more embodiments of the present invention.

FIG. 2A is a photograph showing plants inoculated with the RTI301 strain. FIG. 2B is a photograph showing control plants. These photographs show the positive effects of Bacillus amyloliquefaciens strain RTI301 on early plant growth in wheat according to one or more embodiments of the present invention. The extracted plants after 13 days growth are shown in the figures.

FIG. 3A is a photograph showing plants inoculated with RTI301. FIG. 3B is a photograph showing control plants. These photographs show the positive effects of Bacillus amyloliquefaciens strain RTI301 on growth in wheat after 28 days according to one or more embodiments of the present invention.

FIG. 4 is a bar graph showing the % disease control (mean) on the y axis 10 days after infection with bean rust (Uromyces appendiculatus) following treatment with each of: RTI301 spores in Spent Fermentation Broth (SFB) (applied at 1×10⁸ cfu/ml), SERENADE OPTIMUM (applied at 1×10⁸ cfu/ml), TACTIC (applied at 0.1875% to all formulations and also used as a blank control), SERENADE OPTIMUM (applied at 4×10⁸ cfu/ml), Tebuconazole (applied at 50 g a.i./ha), and Chlorothalonil (applied at 500 g a.i./ha), according to one or more embodiments of the present invention. The non-treated controls resulted in 23% disease. Values followed by the same letter are not significantly different (p=0.10).

FIG. 5 is a bar graph showing the % disease dontrol (mean) on the y axis 10 days after infection with increasing amounts of bean rust (Uromyces appendiculatus) (50 k to 300 k conidia/ml) after treatment with each of RTI301 spores in Spent Fermentation Broth (SFB) (applied at 1×10⁸ cfu/ml) and SERENADE OPTIMUM (applied at 1×10⁸ and 4×10⁸ cfu/ml) as compared to TACTIC (applied at 0.1875% to all formulations and also used as a blank control) and Tebuconazole (HORIZON; applied at 50 g a.i./ha) according to one or more embodiments of the present invention. The percent disease in the check controls was 50 k=6%, 100 k=6%, 150 k=15%, 200 k=15%, and 300 k=7%. Values followed by the same letter are not significantly different (p=0.10).

FIG. 6 shows graphs of development in time of the percent of fruits infected with Botrytis cinerea pathogen in the untreated control (“UT”) in each of two independent tomato field trials to determine antagonism of the RTI301 strain against this pathogen according to one or more embodiments of the present invention.

FIG. 7 shows graphs of development in time of the percent of fruits infected with Botrytis cinerea pathogen in the untreated control (“UT”) in each of four independent strawberry field trials to determine antagonism of the RTI301 strain against this pathogen according to one or more embodiments of the present invention.

FIG. 8A shows growth of Fusarium graminearum on a 869 agar plate. FIG. 8B shows growth of Fusarium graminearum on a 869 agar plate in the presence of 20 μl of a RTI301 spore solution containing 1×10⁸ CFU/ml (left) or 1×10⁹ CFU/ml (right), respectively. FIG. 8C shows growth of 20 μl of a B. amyloliquefaciens RTI301 spore solution containing 1×10⁸ CFU/ml (left) or 1×10⁹ CFU/ml (right), respectively, on a 869 agar plate. FIG. 8D shows growth of Fusarium graminearum on a 869+1% FRACTURE agar plate. FIG. 8E shows growth of Fusarium graminearum on a 869+1% FRACTURE agar plate in the presence of 20 μl of a RTI301 spore solution containing 1×10⁸ CFU/ml (left) or 1×10⁹ CFU/ml (right), respectively. FIG. 8F shows growth of 20 μl of a B. amyloliquefaciens RTI301 spore solution containing 1×10⁸ CFU/ml (left) or 1×10⁹ CFU/ml (right), respectively, on a 869+1% FRACTURE agar plate. These figures show images of a plate assay showing control of Fusarium graminearum by B. amyloliquefaciens RTI301 in the presence and absence of FRACTURE according to one or more embodiments of the present invention.

FIG. 9A shows growth of Fusarium oxysporum fc. cubense on a 869 agar plate. FIG. 9B shows growth of Fusarium oxysporum fc. cubense on a 869 agar plate in the presence of 20 μl of a RTI301 spore solution containing 1×10⁸ CFU/ml (left) or 1×10⁹ CFU/ml (right), respectively. FIG. 9C shows growth of 20 μl of a B. amyloliquefaciens RTI301 spore solution containing 1×10⁸ CFU/ml (left) or 1×10⁹ CFU/ml (right), respectively, on a 869 agar plate. FIG. 9D shows growth of Fusarium oxysporum fc. cubense on a 869+1% FRACTURE agar plate. FIG. 9E shows growth of Fusarium oxysporum fc. cubense on a 869+1% FRACTURE agar plate in the presence of 20 μl of a RTI301 spore solution containing 1×10⁸ CFU/ml (left) or 1×10⁹ CFU/ml (right), respectively. FIG. 9F shows growth of 20 μl of a B. amyloliquefaciens RTI301 spore solution containing 1×10⁸ CFU/ml (left) or 1×10⁹ CFU/ml (right), respectively, on a 869+1% FRACTURE agar plate. These figures show images of a plate assay showing control of Fusarium oxysporum fc. cubense by B. amyloliquefaciens RTI301 in the presence and absence of FRACTURE according to one or more embodiments of the present invention.

FIG. 10 is a schematic diagram showing both previously reported Fengycin-type and Dehydroxyfengycin-type cyclic lipopeptides produced by microbial species including Bacillus amyloliquefaciens and newly identified (shown in bold type) Fengycin- and Dehydroxyfengycin-type molecules produced by the Bacillus amyloliquefaciens RTI301 isolate according to one or more embodiments of the present invention.

FIG. 11 is a graph showing the percentage of recovered lipopeptides from RTI301 spent fermentation broth (SFB) after acid precipitation according to one or more embodiments of the present invention. The terms “301-AP-Pellet” and “301-AP-Supernatant” refer to the resuspended pellet and supernatant, respectively, obtained after acid precipitation plus centrifugation of the SFB. The percentage was calculated and compared based on the integrated ion abundance of each lipopeptide from the RTI301 spent fermentation broth (301-SFB).

### DETAILED DESCRIPTION

The terms “a,” “an,” and “the” refer to “one or more” when used in this application, including the claims. Thus, for example, reference to “a plant” includes a plurality of plants, unless the context clearly is to the contrary.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and claims, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

For the purposes of this specification and claims, the terms “metabolite” and “compound” are used interchangeably when used in connection with compounds having antimicrobial activity that are produced by the RTI301 strain or other Bacillus amyloliquefaciens strains.

For the purposes of this specification and claims, the phrase “a biologically pure culture of a bacterial strain” refers to one or a combination of: spores of the biologically pure fermentation culture of a bacterial strain, vegetative cells of the biologically pure fermentation culture of a bacterial strain, one or more products of the biologically pure fermentation culture of a bacterial strain, a culture solid of the biologically pure fermentation culture of a bacterial strain, a culture supernatant of the biologically pure fermentation culture of a bacterial strain, an extract of the biologically pure fermentation culture of the bacterial strain, and one or more metabolites of the biologically pure fermentation culture of a bacterial strain.

In certain embodiments of the present invention, compositions and methods are provided that include a biologically pure culture of a newly identified strain of Bacillus amyloliquefaciens for application to a plant for one or both of benefiting plant growth or conferring protection against a pathogenic infection in a susceptible plant. In the compositions and methods of the present invention, the growth benefit of the plant is exhibited by improved seedling vigor, improved root development, improved plant growth, improved plant health, increased yield, improved appearance, improved resistance to plant pathogens, reduced pathogenic infection, or a combination thereof.

A plant-associated bacterium, identified as belonging to the species Bacillus amyloliquefaciens, was isolated from the rhizosphere soil of grape vines growing at a vineyard in Long Island, N.Y. and subsequently tested for plant pathogen antagonistic properties. More specifically, the isolated bacterial strain was identified as a new strain of Bacillus amyloliquefaciens through sequence analysis of highly conserved 16S rRNA and rpoB genes (see EXAMPLE 1). The 16S RNA sequence of the new bacterial isolate (designated “Bacillus amyloliquefaciens RTI301”) was determined to be identical to the 16S rRNA gene sequence of three other known strains of Bacillus amyloliquefaciens, Bacillus amyloliquefaciens strain NS6 (KF177175), Bacillus amyloliquefaciens strain FZB42 (NR_075005), and Bacillus subtilis subsp. subtilis strain DSM 10 (NR_027552). It was also determined that the rpoB gene sequence of RTI301 has sequence similarity to the same gene in Bacillus amyloliquefaciens subsp. plantarum TrigoCor1448 (CP007244) (99% sequence identity; 3 base pair difference); Bacillus amyloliquefaciens subsp. plantarum AS43.3 (CP003838) (99% sequence identity; 7 base pair difference); Bacillus amyloliquefaciens CC178 (CP006845) (99% sequence identity; 8 base pair difference), and Bacillus amyloliquefaciens FZB42 (CP000560) (99% sequence identity; 8 base pair difference). The RTI301 strain was identified as a Bacillus amyloliquefaciens, however, the differences in sequence for the rpoB gene at the DNA level indicate that RTI301 is a new strain of Bacillus amyloliquefaciens. The strain of Bacillus amyloliquefaciens RTI301 was deposited on 17 Apr. 2014 under the terms of the Budapest Treaty on the International Recognition of the Deposit of Microorganisms for the Purposes of Patent Procedure at the American Type Culture Collection (ATCC) in Manassas, Va., USA and bears the Patent Accession No. PTA-121165.

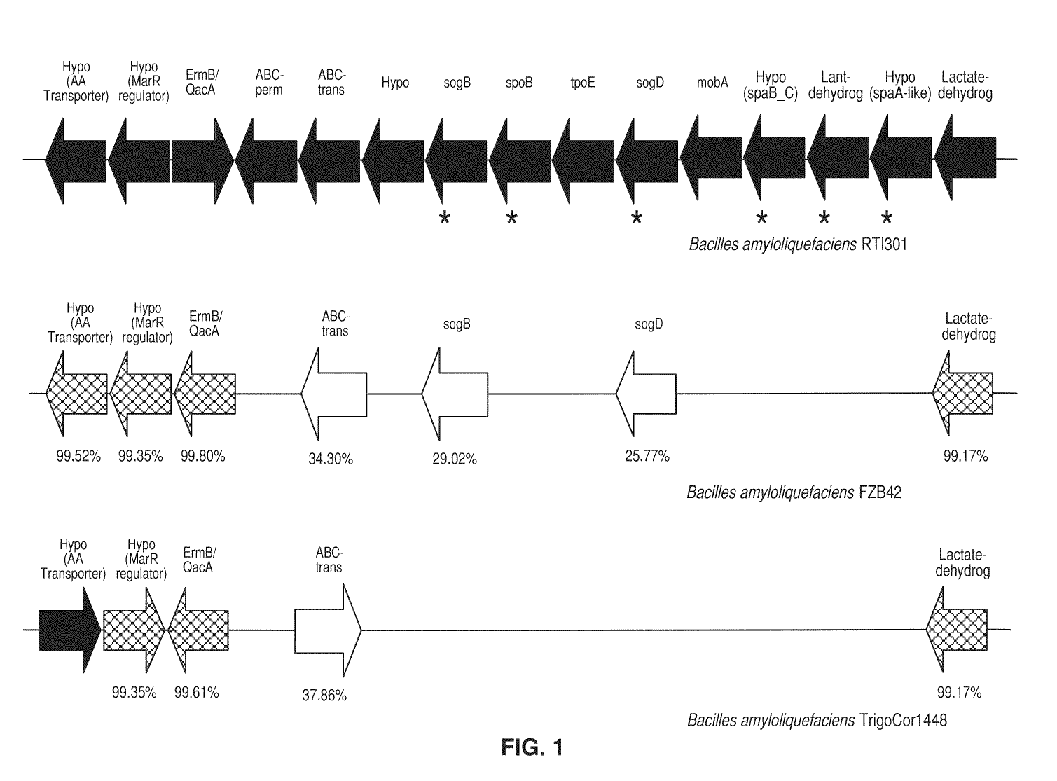

Further sequence analysis of the genome of the Bacillus amyloliquefaciens RTI301 strain revealed that the strain has genes related to lantibiotic biosynthesis for which homologues are lacking in the other closely related Bacillus amyloliquefaciens strains (see EXAMPLE 2). This is illustrated in FIG. 1 which shows a schematic diagram of the genomic organization of the unique lantibiotic biosynthetic cluster found in Bacillus amyloliquefaciens RTI301 and the corresponding region for two known Bacillus amyloliquefaciens reference strains, FZB42 (middle) and TrigoCor1448 (bottom), shown below the RTI301 strain. It can be observed from FIG. 1 that FZB42 and TrigoCor1448 strains lack many of the genes present in this cluster, and there is a low degree of sequence identity within a number of the genes that are present. BLASTn analysis of this cluster against the non-redundant (nr) nucleotide database at NCBI showed high homology to the 5′ and 3′ flanking regions (analogous to the high % similarity in FIG. 1) to B. amyloliquefaciens strains. However, the lantipeptide biosynthetic cluster was unique to RTI301, and no significant homology to any previously sequenced DNA in the NCBI nr database was observed. The data indicate that the newly identified RTI301 has a unique lantibiotic biosynthesis pathway.

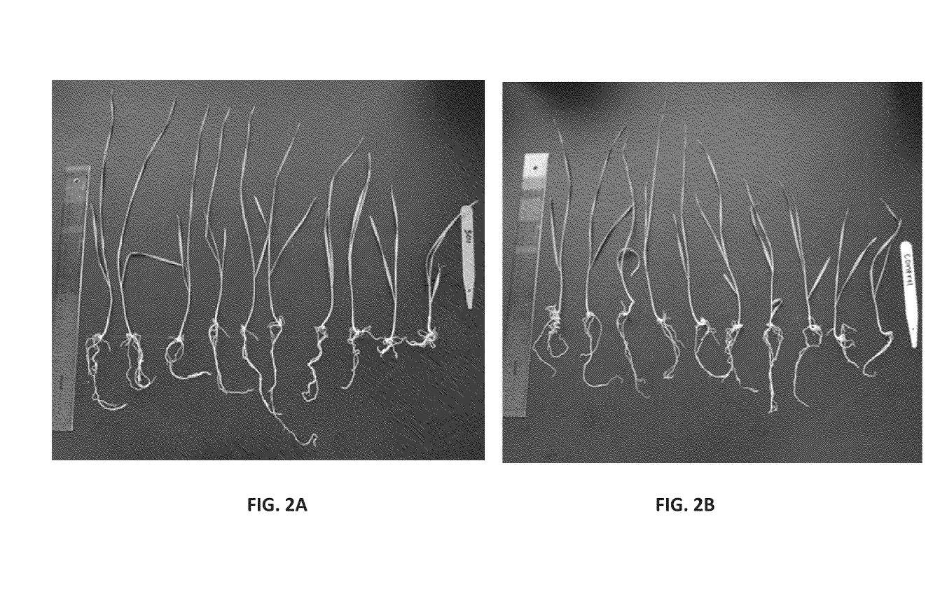

Experiments were performed to determine the growth promoting and antagonisitic activities of the Bacillus amyloliquefaciens RTI301 strain in various plants and under varying conditions. The experimental results are provided in FIGS. 2-11 and in EXAMPLES 3-16 herein that show the ability of the Bacillus amyloliquefaciens RTI301 to benefit plant growth and confer protection against or control plant pathogenic infection as compared to commercially available SERENADE (BAYER CROP SCIENCE, INC) that contains as an active ingredient Bacillus subtilis strain QST713. The experimental results also provide a comparison of the benefits of the new RTI301 strain to commercially available chemical fungicides/bacteriocides. In some cases, application of the Bacillus amyloliquefaciens RTI301 strain alone performed as well as application of a chemical fungicide/bacteriocide. The experimental results also provide an example of the benefits of the new RTI301 strain to enhance the antagonistic properties of FRACTURE (CONSUMO EM VERDE (CEV), BIOTECNOLOGIA DAS PLANTAS S.A., PORTUGAL), a plant extract which contains the BLAD polypeptide as an active ingredient.

In one embodiment of the present invention, a composition is provided that includes a biologically pure culture of Bacillus amyloliquefaciens RTI301 deposited as ATCC No. PTA-121165, or a mutant thereof having all the identifying characteristics thereof, for application to a plant for one or both of benefiting plant growth or conferring protection against a pathogenic infection in a susceptible plant.

In another embodiment, a method is provided for one or both of benefiting growth of a plant or conferring protection against pathogenic infection in a susceptible plant, the method including delivering a composition comprising a biologically pure culture of Bacillus amyloliquefaciens RTI301 deposited as ATCC No. PTA-121165, or a mutant thereof having all the identifying characteristics thereof to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium, in an amount suitable to benefit the plant growth and/or to confer protection against pathogenic infection in the susceptible plant.

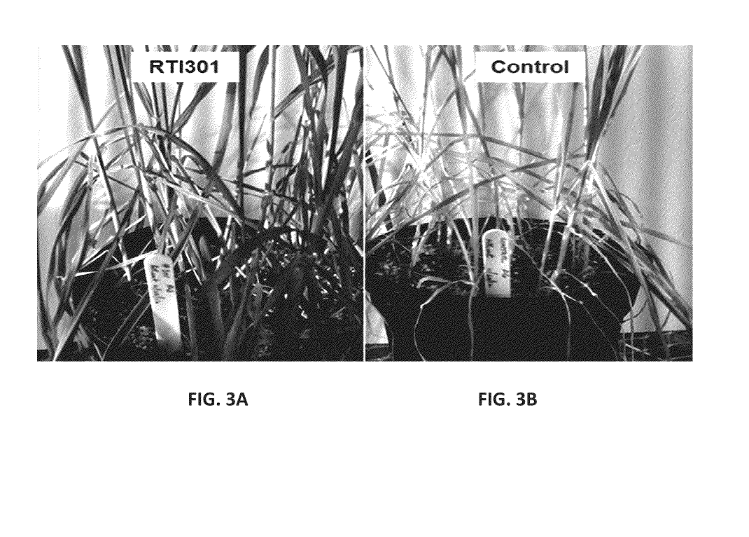

The growth promoting activity of the RTI301 isolate in wheat is described in EXAMPLE 3. Germinated wheat seeds were inoculated for 2 days in a suspension of ˜2×10⁷ CFU/ml of the RTI301 strain and subsequently planted in pots. Photographs of the extracted plants after 13 days growth are shown in FIG. 2. FIG. 2A shows plants inoculated with RTI301 and FIG. 2B shows control plants. Dry weight of the wheat seedlings was determined resulting in an 8.1% increase in dry weight over the non-inoculated control for the RTI301 treated plants. In addition, the beneficial effects of the Bacillus amyloliquefaciens RTI301 strain on early growth in wheat are shown in the images in FIG. 3. FIG. 3A shows 28 day-old wheat plants inoculated with RTI301 and FIG. 3B shows control plants.

The antagonistic properties of the Bacillus amyloliquefaciens RTI301 against several major plant pathogens in plate assays are described in EXAMPLE 4 and phenotypic traits such as phytohormone production, acetoin and indole acetic acid (IAA), and nutrient cycling of the strain are described in EXAMPLE 5.

Beneficial plant associated bacteria, both rhizospheric and endophytic, are known to provide a multitude of benefits to host plants that ranges from resistance to diseases and insects pests and tolerance to environmental stresses including cold, salinity and drought stress. As the plants with inoculated plant growth promoting bacteria acquire more water and nutrient from soils, e.g. due to a better developed root system, the plants grow healthier and are less susceptible to biotic and abiotic stresses. As such the microbial compositions of the present invention can be applied alone or in combination with current crop management inputs such as chemical fertilizers, herbicides, and pesticides to maximize crop productivity. Plant growth promoting effects translate into faster growing plants and increase above ground biomass, a property that can be applied to improve early vigor. One benefit of improved early vigor is that plants are more competitive and out-compete weeds, which directly reduces the cost for weed management by minimizing labor and herbicide application. Plant growth promoting effects also translate into improved root development, including deeper and wider roots with more fine roots that are involved in the uptake of water and nutrients. This property allows for better use of agricultural resources, and a reduction in water used in irrigation needs and/or fertilizer application. Changes in root development and root architecture affect the interactions of the plant with other soil-borne microorganisms, including beneficial fungi and bacteria that help the plant with nutrient uptake including nitrogen fixation and phosphate solubilization. These beneficial microbes also compete against plant pathogens to increase overall plant health and decrease the need for chemical fungicides and pesticides.

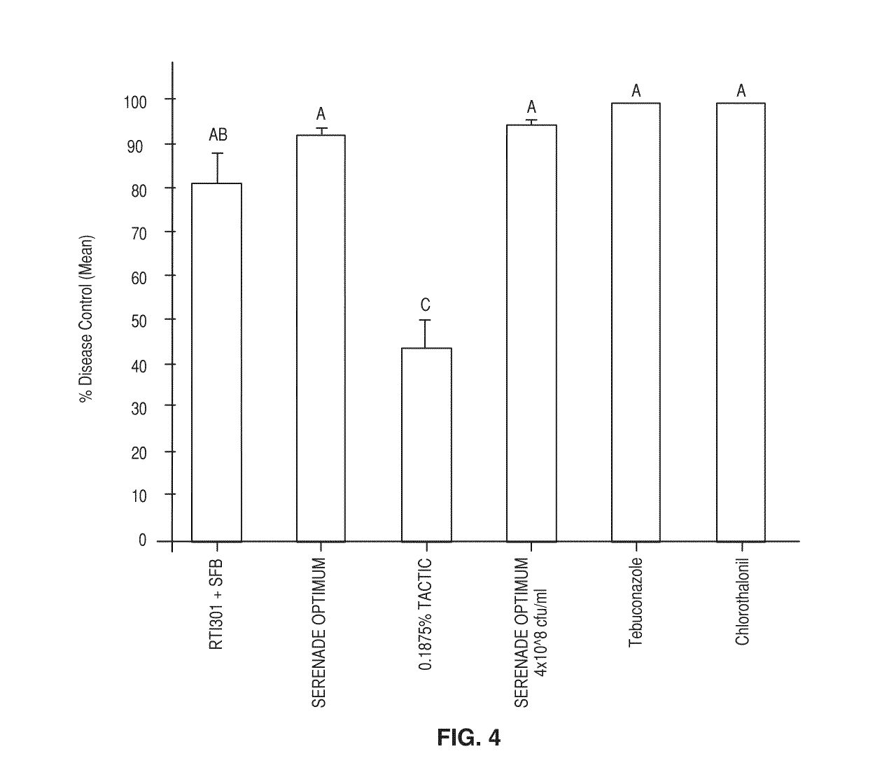

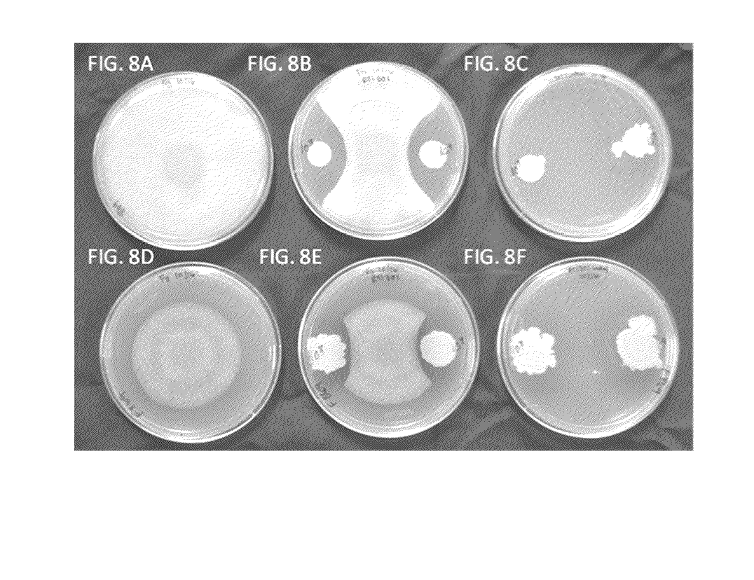

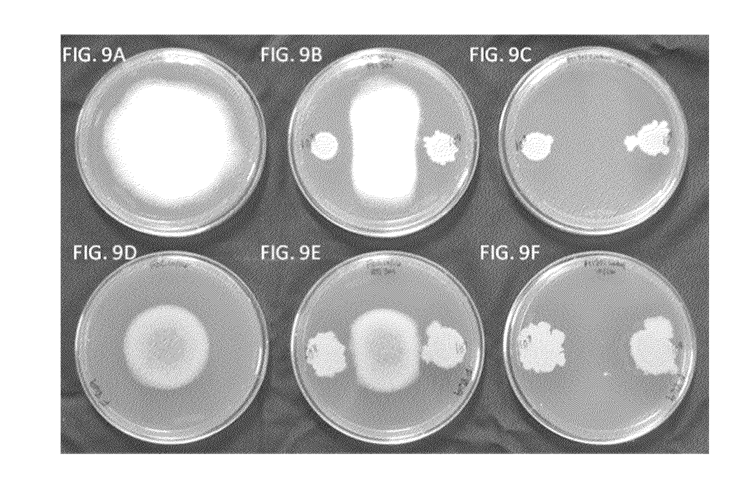

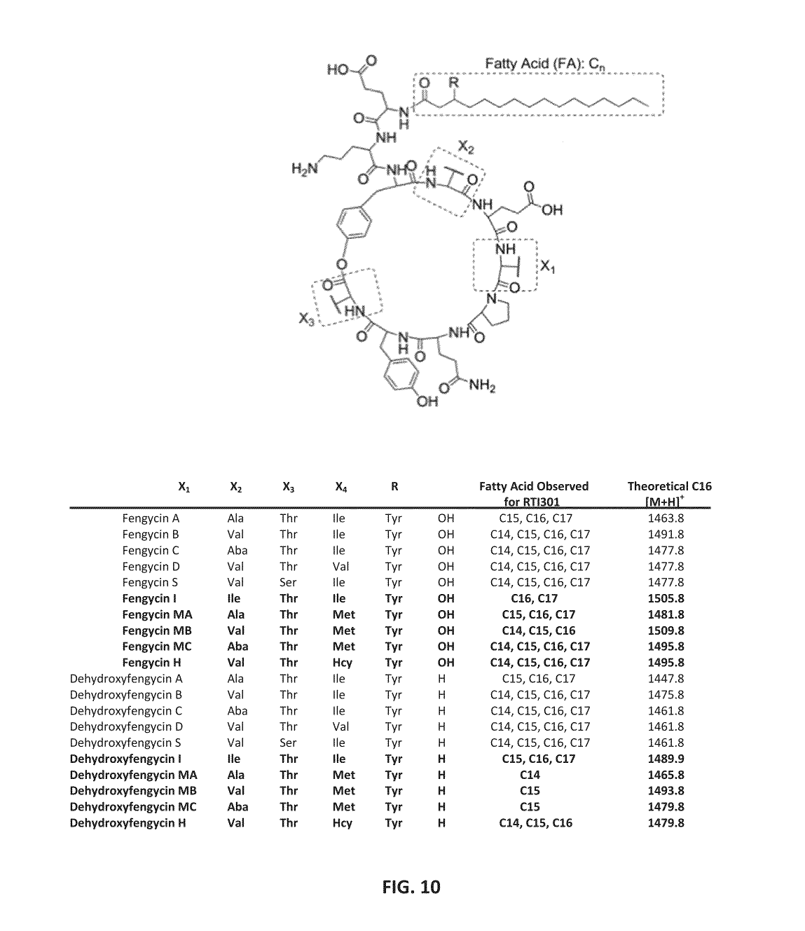

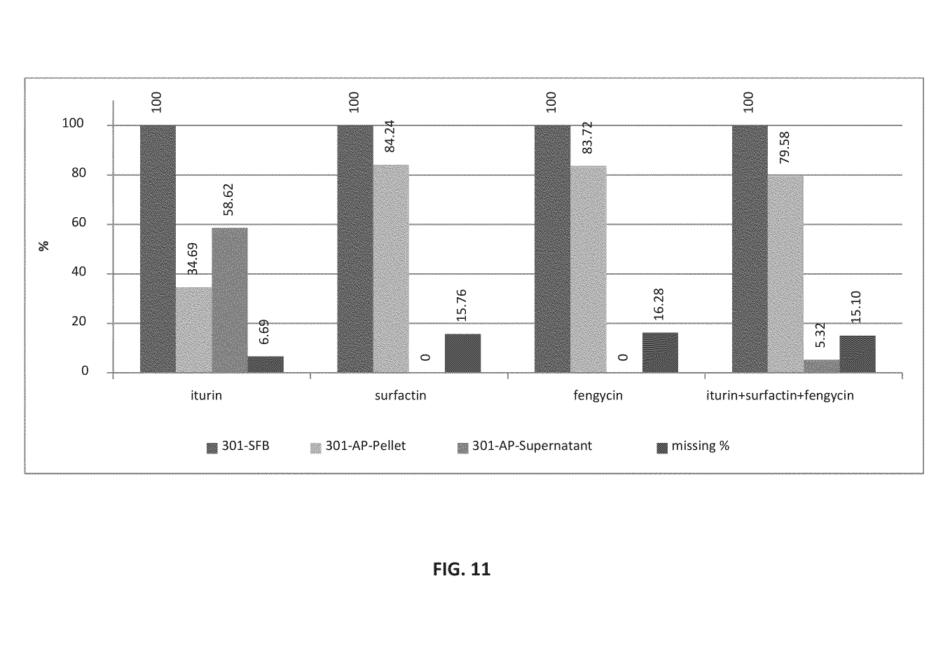

In addition, studies were performed in the greenhouse and in field trials on various crops to determine the ability of the B. amyloliquefaciens RTI301 strain to prevent and/or ameliorate the effects of natural or artificial infection of the plants by a number of common plant pathogens. The results are described in EXAMPLES 6-13 and in FIGS. 4-7. Additional studies describe the antagonistic effects of RTI301 against fungal pathogens in combination with a product containing an antifungal polypeptide (EXAMPLE 14; FIGS. 8-9), and antimicrobial metabolites produced by the RTI301 strain are identified and isolated in EXAMPLES 15 and 16 and FIGS. 10 and 11, respectively.

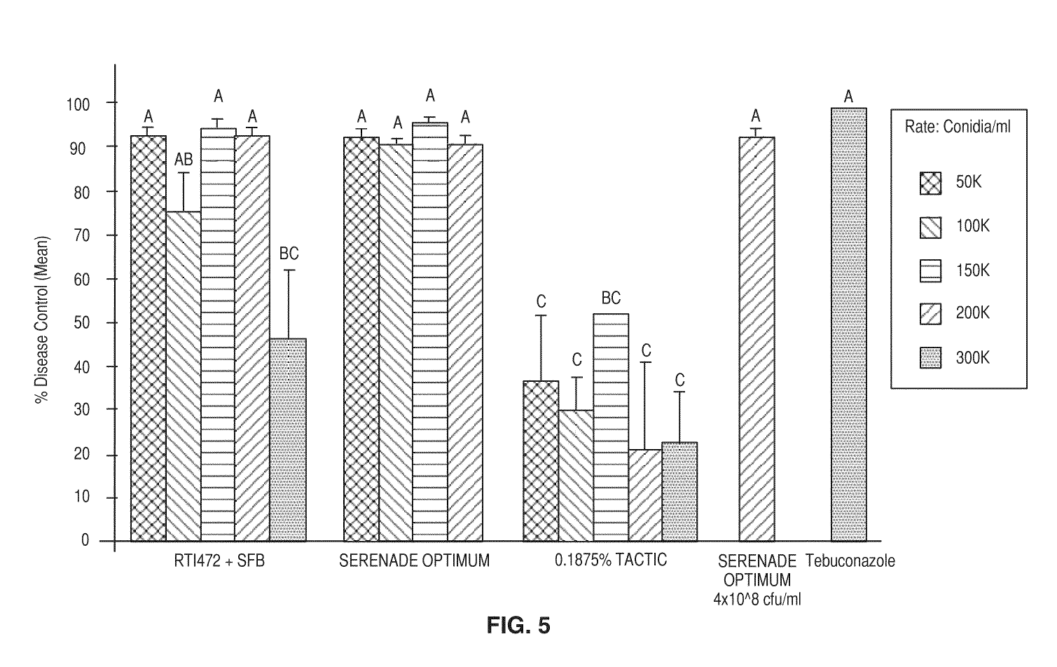

EXAMPLE 6 describes the ability of the B. amyloliquefaciens RTI301 strain to ameliorate the effects of the plant pathogen bean rust (Uromyces appendiculatus) and the plant pathogen Pepper Botrytis Blight (Botrytis cinerea). In a first set of experiments, different formulations of the B. amyloliquefaciens RTI301 strain were tested for foliar application of the RTI301 strain to control Uromyces appendiculatus and Botrytis cinerea. The experimental design was set up such that nine days after infection with the pathogen, the percent of disease control was evaluated for each of: RTI301 spores in Spent Fermentation Broth diluted with water alone (“RTI301+1% SFB”), RTI301 spores in Spent Fermentation Broth diluted with water plus yeast extract (“RTI301+1% SFB+Yeast Extract”), BRAVO WEATHER STIK (500 g a.i./ha Chlorothalonil), HORIZON (50 g a.i./ha Tebuconazole), and SERENADE OPTIMUM at the same spore concentration as the RTI301 strain. The non-treated control (water only) resulted in 28% disease. The results for the Bean Rust and Pepper Botrytis Blight experiments were similar. The results for the Bean Rust experiment are shown in Table III and indicate that the addition of the yeast extract to aid growth of the RTI301 strain on the plant foliage resulted in about a 40% increase in disease control as compared to the RTI301 strain applied without the addition of yeast extract. The amount of disease control exhibited by RTI301+1% SFB+Yeast Extract was similar to that observed for SERENADE OPTIMUM when applied at the same rate (i.e., 1×10⁸ cfu/ml) even though the amount of SFB in the RTI301 formulation was relatively low at 1%, and the SFB can be expected to contain secreted metabolites having antifungal activity. Similar experiments are described in EXAMPLE 6 for disease control of bean rust by the RTI301 strain and the results are shown in Tables IV-V and FIGS. 4-5. The results show comparable control of Bean Rust (Uromyces appendiculatus) after treatment of the bean plant foliage with RTI301 spores as compared to treatment with SERENADE OPTIMUM when applied at the same rate.

Studies were performed in field trials on various crops to determine the ability of the B. amyloliquefaciens RTI301 strain to prevent and/or ameliorate the effects of natural or artificial infection of the plants by a number of common plant pathogens. The results are described in EXAMPLES 7-9. EXAMPLES 7 and 8 describe the control of the plant pathogens powdery mildew on cucumber and Xanthomonas in tomato, respectively. In the trials, the RTI301 was applied to the the crop at the same rate as SERENADE OPTIMUM. Applications were performed 1 to 6 times with 5 to 7 day intervals between applications depending on the crop. The timing of the first application depended on the particular crop and ranged from at the time of planting, a fews weeks after crop emergence, at the beginning of flowering, upon disease emergence, or prior to expectation of disease emergence. The results in EXAMPLE 7 and Table VI show comparable control of powdery mildew in cucumber by RTI301 as compared to SERENADE OPTIMUM when applied at the same rate as a stand-alone biofungicide.

The results in EXAMPLE 8 and Table VII show comparable control of bacterial spot (Xanthomonas) in tomato by RTI301 as compared to SERENADE OPTIMUM when applied at the same rate as a standalone biofungicide. RTI301 as a stand-alone biofungicide showed similar performance as the program using a combination of copper hydroxide and chlorothalonil.

The results described in EXAMPLE 9 and shown in Table VIII show improved or comparable control of wheat head scab, soybean rust, corn rust, cucumber powdery mildew, and bacterial spot on tomato by RTI301 as compared to SERENADE OPTIMUM when applied at the same rate as a stand-alone biofungicide.

The experiments described in EXAMPLE 10 and results shown in Table IX show improved control of Sudden Death Syndrome in soybean by RTI301 in combination with chemical active agents as compared to seeds treated only with the chemical active agents, CRUISERMAXX (SYNGENTA CROP PROTECTION, INC) plus thiophanate methyl fungicide (which combination is referred to herein as “CHEM Control”).

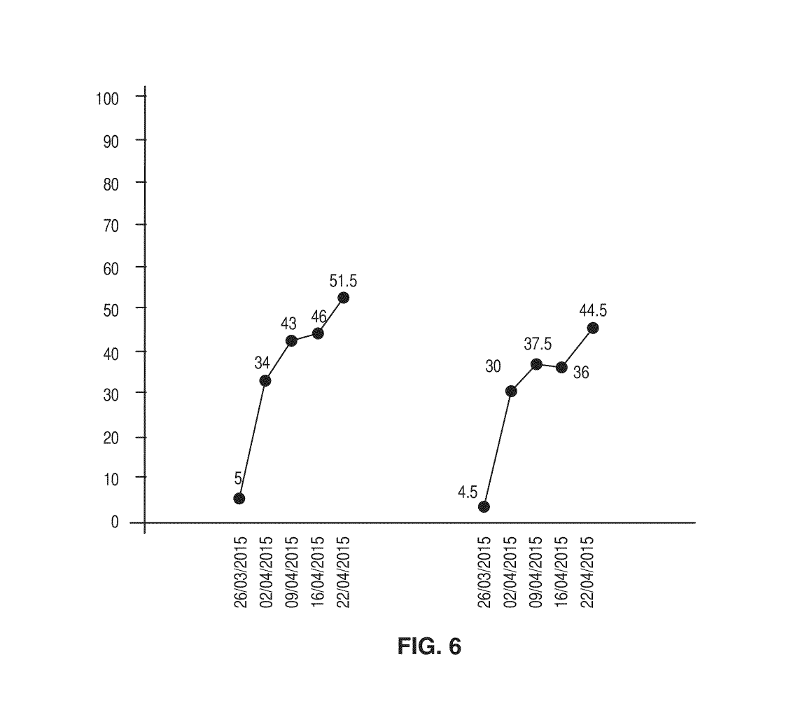

EXAMPLE 11 describes studies performed in field trials of tomato to determine the ability of the B. amyloliquefaciens RTI301 strain to prevent and/or ameliorate the effects of the plant pathogen Brownish Grey Mildew (Botrytis cinerea). The RTI301 strain was compared to application of a combination of chemical active agents referred to as the “FARMER's program” and application of SERENADE MAX having a 10-folder higher concentration of Bacillus subtilis strain QST713 than the RTI301. The development in time of the percent of fruits infected with Botrytis cinerea pathogen in the untreated control (“UT”) in each of the tomato trials is shown in the graphs in FIG. 6. The results in Table X show that the best control of Brownish Grey Mildew on tomatoes was observed for B. amyloliquefaciens RTI301 and the FARMER's program, and outperformed the treatment using SERENADE MAX.

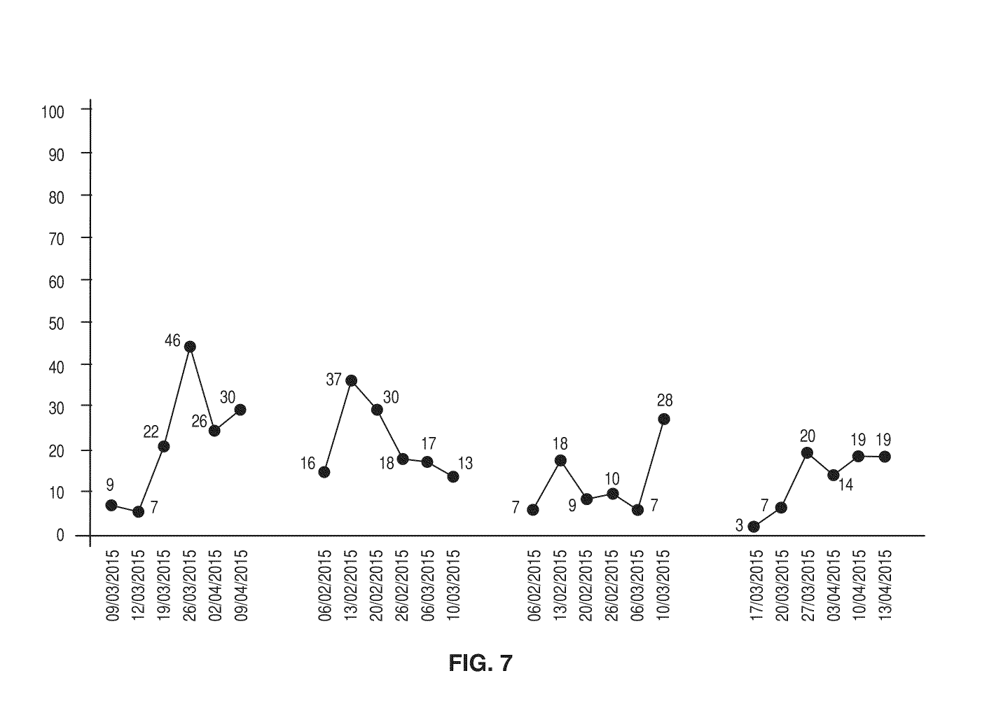

EXAMPLE 12 describes studies performed in field trials of strawberry to determine the ability of the B. amyloliquefaciens RTI301 strain to prevent and/or ameliorate the effects of the plant pathogen Brownish Grey Mildew (Botrytis cinerea). The RTI301 strain was compared to application of a combination of chemical active agents referred to as the “FARMER's program” and application of SERENADE MAX having a 10-folder higher concentration of Bacillus subtilis strain QST713 than the RTI301 strain. The development in time of the percent of fruits infected with Botrytis cinerea pathogen in the untreated control (“UT”) in each of the strawberry trials is shown in the graphs in FIG. 7. The results in Table XI show that improved control of Brownish Grey Mildew on strawberry over the untreated control was observed for all three treatments, B. amyloliquefaciens RTI301, SERENADE MAX, and the FARMER's program, with a slightly higher numerical increase of yield for the treatment with RTI301.

EXAMPLE 13 describes field trials in corn to investigate the effect on plant growth and development after treatment of the plant seed with B. amyloliquefaciens RTI301 strain. The experiment was set up as follows: 1) seed was untreated; 2) seed was treated with a combination of MAXIM, APRON XL, and PONCHO referred to as “CHEM CONTROL”; and 3) seed was treated with CHEM CONTROL plus inoculated with 5.0×10⁺⁵ cfu/seed of strain RTI301. Three field trials were performed in which one had natural disease pressure, one had soil artificially inoculated with Fusarium graminearum, and one had soil artificially inoculated with Rhizoctonia. Notably, in the Rhizoctonia trail, a very large yield benefit of 40.1 bushels per acre was observed for RTI301 plus chemical control over the chemical control alone. In summary, treatment with the chemical control plus RTI301 resulted in an increase in yield for all 3 trials and resulted in a very large increase in yield for the trials inoculated with Rhizoctonia (see Table XII).

EXAMPLE 14 describes an in vitro plate assay that shows the ability of the B. amyloliquefaciens RTI301 strain to enhance the performance of a product sold as FRACTURE to control fungal phytopathogens. The FRACTURE product, a plant extract, contains a polypeptide (BLAD polypeptide) as active ingredient that acts on susceptible fungal pathogens by causing damage to the fungal cell wall and disrupting the inner cell membrane. For the assay, the RTI301 bacterial isolate was grown side by side with pathogenic fungi on agar plates in the presence and absence of 1% FRACTURE. The results of the assays are shown in FIGS. 8A-8F and FIGS. 9A-9F for the plant pathogens Fusarium graminearum and Fusarium oxysporum fc. Cubense, respectively. While addition of 1% FRACTURE to the agar resulted in reduced growth of both pathogens, full inhibition of fungal growth was not achieved. The presence of 1% FRACTURE in the agar medium did not inhibit the growth of B. amyloliquefaciens RTI301. The presence of B. amyloliquefaciens RTI301 in combination with FRACTURE did, however, result in additional inhibition of fungal growth for both Fusarium graminearum and Fusarium oxysporum fc. cubense. Therefore, B. amyloliquefaciens RTI301 can be used to enhance the performance of FRACTURE.

EXAMPLE 15 describes the investigation of the cyclic lipopeptides, Fengycins and Dehydroxyfengycins, produced by the Bacillus amyloliquefaciens RTI301 strain, and surprisingly, the identification of several previously unreported classes of these molecules. It was determined that Bacillus amyloliquefaciens RTI301 produces the previously reported Fengycin A, B and C compounds and the Dehydroxyfengycin A, B and C compounds. Unexpectedly, in addition to these known compounds, it was determined that the RTI301 strain also produces previously unidentified derivatives of these compounds where the L-isoleucine at position 8 of the cyclic peptide chain (referred to as X₃ in FIG. 10) is replaced by L-methionine. The new classes of Fengycin and Dehydroxyfengycin are referred to herein as MA, MB and MC, referring to derivatives of classes A, B and C in which the L-isoleucine at X₃ in FIG. 10 has been replaced by L-methionine. The newly identified molecules are shown in FIG. 10 and in Table XIII. It was further determined that the RTI301 strain produces an additional class of Fengycin and Dehydroxyfengycin that has not been previously identified. In this class, the L-isoleucine of Fengycin B and Dehydroxyfengycin B (position X₃ in FIG. 10) is replaced by L-homo-cysteine (Hcy). These previously unidentified Fengycin and Dehydroxyfengycin metabolites are referred to herein as Fengycin H and Dehydroxyfengycin H and are shown in FIG. 10 and Table XIII. It was further determined that the RTI301 strain produces an additional previously unidentified class of Fengycin and Dehydroxyfengycin metabolites. In this class, the amino acid at position 4 of the cyclic peptide backbone structure (position X₁ in FIG. 10) is replaced by L-isoleucine. These previously unidentified metabolites are referred to herein as Fengycin I and Dehydroxyfengycin I and are shown in FIG. 10 and in Table XIII.

EXAMPLE 16 describes the isolation of antagonistic lipopeptides from B. amyloliquefaciens strain RTI301 spent fermentation broth and an in vitro plate assay showing that the isolated lipopeptides retain their activity against two common plant pathogens. The RTI301 was cultured and an acid precipitate of the culture supernatant was analyzed by LCMS to compare the relative abundance of the iturins, surfactins, and fengycins. FIG. 11 is a graph showing the percentage of recovered lipopeptides from the RTI301 spent fermentation broth after the acid precipitation. The results show that 80% of the total amount of lipopeptides was recovered by acid precipitation. Next, a plate bioassay was performed with the same samples analyzed by LCMS against Botrytis cinerea and Fusarium graminearum. The results showed that the acid precipitated sample has a similar level of antagonistic activity as the starting spent fermentation broth against both Botrytis cinerea and Fusarium graminearum.

In one embodiment, a composition is provided that includes a biologically pure culture of Bacillus amyloliquefaciens RTI472 deposited as ATCC No. PTA-121166, or a mutant thereof having all the identifying characteristics thereof, for application to a plant for one or both of benefiting plant growth or conferring protection against a pathogenic infection in a susceptible plant. The growth benefit of the plant and/or the conferred protection is exhibited by improved seedling vigor, improved root development, improved plant growth, improved plant health, increased yield, improved appearance, improved resistance to plant pathogens, reduced pathogenic infection, or a combination thereof.

In one embodiment, a composition is provided that includes a biologically pure culture of Bacillus amyloliquefaciens RTI301 deposited as ATCC No. PTA-121165, or a mutant thereof having all the identifying characteristics thereof, for application to a plant for one or both of benefiting plant growth or conferring protection against a pathogenic infection in a susceptible plant. The growth benefit of the plant and/or the conferred protection is exhibited by improved seedling vigor, improved root development, improved plant growth, improved plant health, increased yield, improved appearance, improved resistance to plant pathogens, reduced pathogenic infection, or a combination thereof.

The compositions and methods of the present invention are beneficial to a wide range of plants including, but not limited to, monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hardwood cuttings, Chestnuts, Oak, Maple, sugarcane, and sugarbeet.

In one or more embodiments, the plant can include soybean, bean, snap bean, wheat, cotton, corn, pepper, tomato, potato, cassava, grape, strawberry, banana, peanut, squash, pumpkin, eggplant, and cucumber.

In the compositions and methods of the present invention, the pathogenic infection can be caused by a wide variety of plant pathogens including, for example, but not limited to, a plant fungal pathogen, a plant bacterial pathogen, a rust fungus, a Botrytis spp., a Botrytis cinerea, a Botrytis squamosa, an Erwinia spp., an Erwinia carotovora, an Erwinia amylovora, a Dickeya spp., a Dickeya dadantii, a Dickeya solani, an Agrobacterium spp., a Agrobacterium tumefaciens, a Xanthomonas spp., a Xanthomonas axonopodis, a Xanthomonas campestris pv. carotae, a Xanthomonas pruni, a Xanthomonas arboricola, a Xanthomonas oryzae pv. oryzae, a Xylella spp., a Xylella fastidiosa, a Candidatus spp., a Candidatus liberibacter, a Fusarium spp., a Fusarium colmorum, a Fusarium graminearum, a Fusarium oxysporum, a Fusarium oxysporum f. sp. Cubense, a Fusarium oxysporum f. sp. Lycopersici, a Fusarium virguliforme, a Sclerotinia spp., a Sclerotinia sclerotiorum, a Sclerotinia minor, Sclerotinia homeocarpa, a Cercospora/Cercosporidium spp., an Uncinula spp., an Uncinula necator (Powdery Mildew), a Podosphaera spp. (Powdery Mildew), a Podosphaera leucotricha, a Podosphaera clandestine, a Phomopsis spp., a Phomopsis viticola, an Alternaria spp., an Alternaria tenuissima, an Alternaria porri, an Alternaria alternate, an Alternaria solani, an Alternaria tenuis, a Pseudomonas spp., a Pseudomonas syringae pv. Tomato, a Phytophthora spp., a Phytophthora infestans, a Phytophthora parasitica, a Phytophthora sojae, a Phytophthora capsici, a Phytophthora cinnamon, a Phytophthora fragariae, a Phytophthora spp., a Phytophthora ramorum, a Phytophthora palmivara, a Phytophthora nicotianae, a Phakopsora spp., a Phakopsora pachyrhizi, a Phakopsora meibomiae an Aspergillus spp., an Aspergillus flavus, an Aspergillus niger, a Uromyces spp., a Uromyces appendiculatus, a Cladosporium spp., a Cladosporium herbarum, a Rhizopus spp., a Rhizopus arrhizus, a Penicillium spp., a Rhizoctonia spp., a Rhizoctonia solani, a Rhizoctonia zeae, a Rhizoctonia oryzae, a Rhizoctonia caritae, a Rhizoctonia cerealis, a Rhizoctonia crocorum, a Rhizoctonia fragariae, a Rhizoctonia ramicola, a Rhizoctonia rubi, a Rhizoctonia leguminicola, a Macrophomina phaseolina, a Magnaorthe oryzae, a Mycosphaerella spp., Mycosphaerella graminocola, a Mycosphaerella fijiensis (Black sigatoga), a Mycosphaerella pomi, a Mycosphaerella citri, a Magnaporthe spp., a Magnaporthe grisea, a Monilinia spp., a Monilinia fruticola, a Monilinia vacciniicorymbosi, a Monilinia laxa, a Colletotrichum spp., a Colletotrichum gloeosporiodes, a Colletotrichum acutatum, a Colletotrichum Candidum, a Diaporthe spp., a Diaporthe citri, a Corynespora spp., a Corynespora Cassiicola, a Gymnosporangium spp., a Gymnosporangium juniperi-virginianae, a Schizothyrium spp., a Schizothyrium pomi, a Gloeodes spp., a Gloeodes pomigena, a Botryosphaeria spp., a Botryosphaeria dothidea, a Neofabraea spp., a Wilsonomyces spp., a Wilsonomyces carpophilus, a Sphaerotheca spp., a Sphaerotheca macularis, a Sphaerotheca pannosa, a Erysiphe spp., a Stagonospora spp., a Stagonospora nodorum, a Pythium spp., a Pythium ultimum, a Pythium aphanidermatum, a Pythium irregularum, a Pythium ulosum, a Pythium lutriarium, a Pythium sylvatium, a Venturia spp, a Venturia inaequalis, a Verticillium spp., a Ustilago spp., a Ustilago nuda, a Ustilago maydis, a Ustilago scitaminea, a Claviceps spp., a Claviceps puprrea, a Tilletia spp., a Tilletia tritici, a Tilletia laevis, a Tilletia horrid, a Tilletia controversa, a Phoma spp., a Phoma glycinicola, a Phoma exigua, a Phoma lingam, a Cocliobolus sativus, a Gaeumanomyces gaminis, a Colleototricum spp., a Rhychosporium spp., Rhychosporium secalis, a Biopolaris spp., a Helminthosporium spp., a Helminthosporium secalis, a Helminthosporium maydis, a Helminthosporium solai, and a Helminthosporium tritici-repentis, or combinations thereof.

In some embodiments, the pathogenic infection can be caused by one or a combination of: Soybean rust fungi (Phakopsora pachyrhizi, Phakopsora meibomiae) and the plant comprises soybean; Botrytis cinerea (Botrytis Blight) and the plant comprises grape; Botrytis cinerea (Botrytis Blight) and the plant comprises strawberry; Botrytis cinerea (Botrytis Blight) and the plant comprises tomato; Alternaria spp. (e.g. A. solani) and the plant comprises tomato; Alternaria spp. (e.g. A. solani) and the plant comprises potato; Bean Rust (Uromyces appendiculatus) and the plant comprises common bean; Microsphaera diffusa (Soybean Powdery Mildew) and the plant comprises soybean; Mycosphaerella fijiensis (Black sigatoga) or Fusarium oxysporum f. sp. cubense (Panama disease) and the plant comprises banana; Xanthomonas spp. or Xanthomonas oryzae pv. oryzae and the plant comprises rice; Xanthomonas axonopodis and the plant comprises cassava; Xanthomonas campestris and the plant comprises tomato; Botrytis cinerea (Pepper Botrytis Blight) and the plant comprises pepper; Powdery mildew and the plant comprises a cucurbit; Sclerotinia sclerotiorum (white mold) and the plant comprises snap bean; Sclerotinia sclerotiorum (white mold) and the plant comprises potato; Sclerotinia homeocarpa (dollar spot) and the plant comprises turfgrass; Southern White Mold and the plant comprises peanut; Leaf spot (Cercospora/Cercosporidium) and the plant comprises peanut; Fusarium graminearum (Wheat Head Scab) and the plant comprises wheat; Mycosphaerella graminicola (Septoria tritici blotch) and the plant comprises wheat; Stagonospora nodorum (glume blotch and septoria nodorum blotch), and the plant compromises wheat; Erwinia amylovora, and the plant compromises apple, pear and other pome fruits; Venturia inaequalis, and the plant compromises apple, pear and other pome fruits; or Rhizoctonia solani and the plant comprises wheat, rice, turfgrass, soybean, corn, legumes and vegetable crops.

The compositions including the RTI301 strain can be in the form of a liquid, an oil dispersion, a dust, a dry wettable powder, a spreadable granule, or a dry wettable granule.

The compositions benefit plant growth when applied to foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium, when applied in an amount suitable to benefit the plant growth and/or to confer protection against pathogenic infection in the susceptible plant.

The compositions including the RTI301 strain can further include one or a combination of a carrier, a surfactant, a dispersant, or a yeast extract. For the purposes of this specification and claims, the terms “surfactant” and “adjuvant” are used interchangeably. The yeast extract can be delivered at a rate for benefiting plant growth ranging from about 0.01% to 0.2% w/w.

The composition can be in the form of a planting matrix. The planting matrix can be in the form of a potting soil.

In one embodiment, a composition is provided for one or both of benefiting plant growth or conferring protection against pathogenic infection in a susceptible plant, the composition including both a biologically pure culture of Bacillus amyloliquefaciens RTI301 deposited as ATCC No. PTA-121165, or a mutant thereof having all the identifying characteristics thereof, in an amount suitable to benefit plant growth and/or to confer protection against pathogenic infection in the susceptible plant; and one or a combination of a microbial, a biological, or a chemical insecticide, fungicide, nematicide, bacteriocide, herbicide, plant extract, plant growth regulator, or fertilizer, in an amount suitable to benefit plant growth and/or to confer protection against pathogenic infection in the susceptible plant. In this embodiment, the biologically pure culture of Bacillus amyloliquefaciens RTI301 and the one or a combination of a microbial or the chemical insecticide, fungicide, nematicide, bacteriocide, herbicide, plant extract, plant growth regulator, or fertilizer are formulated together.

In one embodiment, the fungicide can include an extract from Lupinus albus. In one embodiment, the fungicide can include a BLAD polypeptide. The BLAD polypeptide can be a fragment of the naturally occurring seed storage protein from sweet lupine (Lupinus albus) that acts on susceptible fungal pathogens by causing damage to the fungal cell wall and disrupting the inner cell membrane. The compositions can include about 20% of the BLAD polypeptide.

In the compositions including Bacillus amyloliquefaciens RTI301, the composition can be in the form of a liquid and the Bacillus amyloliquefaciens RTI301 can be present at a concentration of from about 1.0×10⁸ CFU/ml to about 1.0×10¹² CFU/ml. The composition can be in the form of a dust, a dry wettable powder, a spreadable granule, or a dry wettable granule and the Bacillus amyloliquefaciens RTI301 can be present in an amount of from about 1.0×10⁸ CFU/g to about 1.0×10¹² CFU/g. The composition can be the form of an oil dispersion and the Bacillus amyloliquefaciens RTI301 can be present at a concentration of from about 1.0×10⁸ CFU/ml to about 1.0×10¹² CFU/ml. The Bacillus amyloliquefaciens RTI301 can be in the form of spores or vegetative cells.

In one embodiment, a method is provided for one or both of benefiting growth of a plant or conferring protection against pathogenic infection in a susceptible plant, the method including delivering a composition comprising a biologically pure culture of Bacillus amyloliquefaciens RTI301 deposited as ATCC No. PTA-121165, or a mutant thereof having all the identifying characteristics thereof to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium, in an amount suitable to benefit the plant growth and/or to confer protection against pathogenic infection in the susceptible plant. The composition can be delivered to the foliage of the plant.

In the method, the composition including the Bacillus amyloliquefaciens RTI301 can further include one or a combination of a carrier, a surfactant, a dispersant, or a yeast extract. The yeast extract can be delivered at a rate for benefiting plant growth ranging from about 0.01% to 0.2% w/w.

In another embodiment of the present invention, a method is provided for one or both of benefiting growth of a plant or conferring protection against pathogenic infection in a susceptible plant, the method including delivering a composition comprising a biologically pure culture of Bacillus amyloliquefaciens RTI301 deposited as ATCC No. PTA-121165, or a mutant thereof having all the identifying characteristics thereof, in an amount suitable for benefiting the plant growth and/or conferring protection against the pathogenic infection; and one or a combination of a microbial, a biological, or a chemical insecticide, fungicide, nematicide, bacteriocide, herbicide, plant extract, plant growth regulator, or fertilizer in an amount suitable for benefiting the plant growth and/or conferring protection against the pathogenic infection, to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In one embodiment, a method is provided for one or both of benefiting growth of a plant or conferring protection against pathogenic infection in a susceptible plant, the method including delivering a combination of a first composition comprising a biologically pure culture of Bacillus amyloliquefaciens RTI301 deposited as ATCC No. PTA-121165, or a mutant thereof having all the identifying characteristics thereof in an amount suitable to benefit the plant growth and/or to confer protection against pathogenic infection in the susceptible plant; and a second composition comprising one or a combination of a microbial, a biological, or a chemical insecticide, fungicide, nematicide, bacteriocide, herbicide, plant extract, plant growth regulator, or fertilizer, in an amount suitable to benefit the plant growth and/or to confer protection against pathogenic infection in the susceptible plant, to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium. In one embodiment, the first and second compositions can be delivered to the foliage of the plant.

The first composition can further include one or a combination of a carrier, a surfactant, a dispersant, or a yeast extract. The yeast extract can be delivered at a rate for benefiting plant growth ranging from about 0.01% to 0.2% w/w.

The fungicide of the second composition can include an extract from Lupinus albus. The fungicide of the second composition can include a BLAD polypeptide. The BLAD polypeptide can be a fragment of the naturally occurring seed storage protein from sweet lupine (Lupinus albus) that acts on susceptible fungal pathogens by causing damage to the fungal cell wall and disrupting the inner cell membrane. The fungicide of the second composition can include about 20% of a BLAD polypeptide.

In the compositions and methods of the present invention for delivering RTI301 in combination with a microbial, a biological, or a chemical insecticide, fungicide, nematicide, bacteriocide, herbicide, plant extract, plant growth regulator, or fertilizer, the growth benefit of the plant and/or the conferred protection can be exhibited by improved seedling vigor, improved root development, improved plant growth, improved plant health, increased yield, improved appearance, improved resistance to plant pathogens, reduced pathogenic infection, or a combination thereof.

In one embodiment, the method can further include applying a liquid fertilizer to: soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In the methods for delivering RTI301 in combination with a microbial, a biological, or a chemical insecticide, fungicide, nematicide, bacteriocide, herbicide, plant extract, plant growth regulator, or fertilizer, the composition can be in the form of a liquid, an oil dispersion, a dust, a dry wettable powder, a spreadable granule, or a dry wettable granule. The Bacillus amyloliquefaciens RTI301 can be in the form of spores or vegetative cells. The Bacillus amyloliquefaciens RTI301 can be delivered at a rate for benefiting plant growth of about 1.0×10¹⁰ CFU/ha to about 1.0×10¹⁴ CFU/ha. The yeast extract can be delivered at a rate for benefiting plant growth ranging from about 0.01% to 0.2% w/w.

In the compositions and methods of the present invention for delivering RTI301 with a microbial, a biological, or a chemical insecticide, fungicide, nematicide, bacteriocide, herbicide, plant extract, plant growth regulator, or fertilizer, the insecticide can comprise bifenthrin. In one or more embodiments, the nematicide can comprise cadusafos. In one or more embodiments, the insecticide can comprise bifenthrin and clothianidin. In one or more embodiments, the insecticide can comprise bifenthrin and the composition can be formulated as a liquid. In one or more embodiments, the insecticide can comprise bifenthrin and clothianidin and the composition can be formulated as a liquid. In one or more embodiments, the insecticide can comprise bifenthrin or zeta-cypermethrin. In one or more embodiments, the composition can be formulated as a liquid and the insecticide can comprise bifenthrin or zeta-cypermethrin.

The insecticide can be bifenthrin and the composition formulation can further comprise a hydrated aluminum-magnesium silicate, and at least one dispersant selected from the group consisting of a sucrose ester, a lignosulfonate, an alkylpolyglycoside, a naphthalenesulfonic acid formaldehyde condensate and a phosphate ester. The bifenthrin insecticide can be present at a concentration ranging from 0.1 g/ml to 0.2 g/ml. The bifenthrin insecticide can be present at a concentration of about 0.1715 g/ml. The rate of application of the bifenthrin insecticide can be in the range of from about 0.1 gram of bifenthrin per hectare (g ai/ha) to about 1000 g ai/ha, more preferably in a range of from about 1 g ai/ha to about 100 g ai/ha.

In an embodiment, the bifenthrin composition can comprise: bifenthrin; a hydrated aluminum-magnesium silicate; and at least one dispersant selected from a sucrose ester, a lignosulfonate, an alkylpolyglycoside, a naphthalenesulfonic acid formaldehyde condensate and a phosphate ester.

The bifenthrin can be preferably present in a concentration of from 1.0% by weight to 35% by weight, more particularly, from 15% by weight to 25% by weight based upon the total weight of all components in the composition. The bifenthrin insecticide composition can be formulated in a manner suitable for mixture as a liquid with a fertilizer. The bifenthrin insecticide composition can be present in the liquid formulation at a concentration ranging from 0.1 g/ml to 0.2 g/ml. The bifenthrin insecticide may be present in the liquid formulation at a concentration of about 0.1715 g/ml. The terms “can be formulated in a manner suitable for mixture as a liquid with a fertilizer” and “in a formulation compatible with a liquid fertilizer” are herein used interchangeably throughout the specification and claims and are intended to mean that the formulation is capable of dissolution or dispersion or emulsion in an aqueous solution to allow for mixing with a fertilizer for delivery to plants in a liquid formulation.

The dispersant or dispersants can preferably be present in a total concentration of from about 0.02% by weight to about 20% by weight based upon the total weight of all components in the composition.

In some embodiments, the hydrated aluminum-magnesium silicate can be selected from the group consisting of montmorillonite and attapulgite.

In some embodiments, the phosphate ester can be selected from a nonyl phenol phosphate ester and a tridecyl alcohol ethoxylated phosphate potassium salt.

Other embodiments can further include at least one of an anti-freeze agent, an anti-foam agent and a biocide.

In one embodiment a composition is provided for benefiting plant growth, the composition comprising: a biologically pure culture of Bacillus amyloliquefaciens RTI301 deposited as ATCC No. PTA-121165, or a mutant thereof having all the identifying characteristics thereof; and an insecticide. The insecticide can be one or a combination of pyrethroids, bifenthrin, tefluthrin, zeta-cypermethrin, organophosphates, chlorethoxyfos, chlorpyrifos, tebupirimfos, cyfluthrin, fiproles, fipronil, nicotinoids, or clothianidin. The insecticide can include bifenthrin. The composition can be in a formulation compatible with a liquid fertilizer. The composition including bifenthrin can further include a hydrated aluminum-magnesium silicate and at least one dispersant. The bifenthrin insecticide can be present at a concentration ranging from 0.1 g/ml to 0.2 g/ml. The bifenthrin insecticide can be present at a concentration of about 0.1715 g/ml.

In addition, in one or more embodiments, suitable insecticides, herbicides, fungicides, and nematicides of the compositions and methods of the present invention can include the following:

Insecticides: A0) various insecticides, including agrigata, al-phosphide, amblyseius, aphelinus, aphidius, aphidoletes, artimisinin, autographa californica NPV, azocyclotin, Bacillus subtilis, Bacillus thuringiensis-spp. aizawai, Bacillus thuringiensis spp. kurstaki, Bacillus thuringiensis, Beauveria, Beauveria bassiana, betacyfluthrin, biologicals, bisultap, brofluthrinate, bromophos-e, bromopropylate, Bt-Corn-GM, Bt-Soya-GM, capsaicin, cartap, celastrus-extract, chlorantraniliprole, chlorbenzuron, chlorethoxyfos, chlorfluazuron, chlorpyrifos-e, cnidiadin, cryolite, cyanophos, cyantraniliprole, cyhalothrin, cyhexatin, cypermethrin, dacnusa, DCIP, dichloropropene, dicofol, diglyphus, diglyphus+dacnusa, dimethacarb, dithioether, dodecyl-acetate, emamectin, encarsia, EPN, eretmocerus, ethylene-dibromide, eucalyptol, fatty-acids, fatty-acids/salts, fenazaquin, fenobucarb (BPMC), fenpyroximate, flubrocythrinate, flufenzine, formetanate, formothion, furathiocarb, gamma-cyhalothrin, garlic-juice, granulosis-virus, harmonia, heliothis armigera NPV, inactive bacterium, indol-3-ylbutyric acid, iodomethane, iron, isocarbofos, isofenphos, isofenphos-m, isoprocarb, isothioate, kaolin, lindane, liuyangmycin, matrine, mephosfolan, metaldehyde, metarhizium-anisopliae, methamidophos, metolcarb (MTMC), mineral-oil, mirex, m-isothiocyanate, monosultap, myrothecium verrucaria, naled, neochrysocharis formosa, nicotine, nicotinoids, oil, oleic-acid, omethoate, orius, oxymatrine, paecilomyces, paraffin-oil, parathion-e, pasteuria, petroleum-oil, pheromones, phosphorus-acid, photorhabdus, phoxim, phytoseiulus, pirimiphos-e, plant-oil, plutella xylostella GV, polyhedrosis-virus, polyphenol-extracts, potassium-oleate, profenofos, prosuler, prothiofos, pyraclofos, pyrethrins, pyridaphenthion, pyrimidifen, pyriproxifen, quillay-extract, quinomethionate, rape-oil, rotenone, saponin, saponozit, sodium-compounds, sodium-fluosilicate, starch, steinernema, streptomyces, sulfluramid, sulphur, tebupirimfos, tefluthrin, temephos, tetradifon, thiofanox, thiometon, transgenics (e.g., Cry3Bb1), triazamate, trichoderma, trichogramma, triflumuron, verticillium, vertrine, isomeric insecticides (e.g., kappa-bifenthrin, kappa-tefluthrin), dichoromezotiaz, broflanilide, pyraziflumid; A1) the class of carbamates, including aldicarb, alanycarb, benfuracarb, carbaryl, carbofuran, carbosulfan, methiocarb, methomyl, oxamyl, pirimicarb, propoxur and thiodicarb; A2) the class of organophosphates, including acephate, azinphos-ethyl, azinphos-methyl, chlorfenvinphos, chlorpyrifos, chlorpyrifos-methyl, demeton-S-methyl, diazinon, dichlorvos/DDVP, dicrotophos, dimethoate, disulfoton, ethion, fenitrothion, fenthion, isoxathion, malathion, methamidaphos, methidathion, mevinphos, monocrotophos, oxymethoate, oxydemeton-methyl, parathion, parathion-methyl, phenthoate, phorate, phosalone, phosmet, phosphamidon, pirimiphos-methyl, quinalphos, terbufos, tetrachlorvinphos, triazophos and trichlorfon; A3) the class of cyclodiene organochlorine compounds such as endosulfan; A4) the class of fiproles, including ethiprole, fipronil, pyrafluprole and pyriprole; A5) the class of neonicotinoids, including acetamiprid, clothianidin, dinotefuran, imidacloprid, nitenpyram, thiacloprid and thiamethoxam; A6) the class of spinosyns such as spinosad and spinetoram; A7) chloride channel activators from the class of mectins, including abamectin, emamectin benzoate, ivermectin, lepimectin and milbemectin; A8) juvenile hormone mimics such as hydroprene, kinoprene, methoprene, fenoxycarb and pyriproxyfen; A9) selective homopteran feeding blockers such as pymetrozine, flonicamid and pyrifluquinazon; A10) mite growth inhibitors such as clofentezine, hexythiazox and etoxazole; A11) inhibitors of mitochondrial ATP synthase such as diafenthiuron, fenbutatin oxide and propargite; uncouplers of oxidative phosphorylation such as chlorfenapyr; A12) nicotinic acetylcholine receptor channel blockers such as bensultap, cartap hydrochloride, thiocyclam and thiosultap sodium; A13) inhibitors of the chitin biosynthesis type 0 from the benzoylurea class, including bistrifluron, diflubenzuron, flufenoxuron, hexaflumuron, lufenuron, novaluron and teflubenzuron; A14) inhibitors of the chitin biosynthesis type 1 such as buprofezin; A15) moulting disruptors such as cyromazine; A16) ecdyson receptor agonists such as methoxyfenozide, tebufenozide, halofenozide and chromafenozide; A17) octopamin receptor agonists such as amitraz; A18) mitochondrial complex electron transport inhibitors pyridaben, tebufenpyrad, tolfenpyrad, flufenerim, cyenopyrafen, cyflumetofen, hydramethylnon, acequinocyl or fluacrypyrim; A19) voltage-dependent sodium channel blockers such as indoxacarb and metaflumizone; A20) inhibitors of the lipid synthesis such as spirodiclofen, spiromesifen and spirotetramat; A21) ryanodine receptor-modulators from the class of diamides, including flubendiamide, the phthalamide compounds (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, chloranthraniliprole and cy-anthraniliprole; A22) compounds of unknown or uncertain mode of action such as azadirachtin, amidoflumet, bifenazate, fluensulfone, piperonyl butoxide, pyridalyl, sulfoxaflor; or A23) sodium channel modulators from the class of pyrethroids, including acrinathrin, allethrin, bifenthrin, cyfluthrin, lambda-cyhalothrin, cyper-methrin, alpha-cypermethrin, beta-cypermethrin, zeta-cypermethrin, deltamethrin, esfenvalerate, etofenprox, fenpropathrin, fenvalerate, flucythrinate, tau-fluvalinate, permethrin, silafluofen and tralomethrin.

Fungicides: B0) benzovindiflupyr, anitiperonosporic, ametoctradin, amisulbrom, copper salts (e.g., copper hydroxide, copper oxychloride, copper sulfate, copper persulfate), boscalid, thiflumazide, flutianil, furalaxyl, thiabendazole, benodanil, mepronil, isofetamid, fenfuram, bixafen, fluxapyroxad, penflufen, sedaxane, coumoxystrobin, enoxastrobin, flufenoxystrobin, pyraoxystrobin, pyrametostrobin, triclopyricarb, fenaminstrobin, metominostrobin, pyribencarb, meptyldinocap, fentin acetate, fentin chloride, fentin hydroxide, oxytetracycline, chlozolinate, chloroneb, tecnazene, etridiazole, iodocarb, prothiocarb, Bacillus subtilis syn., Bacillus amyloliquefaciens (e.g., strains QST 713, FZB24, MBI600, D747), extract from Melaleuca alternifolia, extract from Lupinus albus doce, BLAD polypeptide, pyrisoxazole, oxpoconazole, etaconazole, fenpyrazamine, naftifine, terbinafine, validamycin, pyrimorph, valifenalate, fthalide, probenazole, isotianil, laminarin, extract from Reynoutria sachalinensis, phosphorous acid and salts, teclofthalam, triazoxide, pyriofenone, organic oils, potassium bicarbonate, chlorothalonil, fluoroimide; B1) azoles, including bitertanol, bromuconazole, cyproconazole, difenoconazole, diniconazole, enilconazole, epoxiconazole, fluquinconazole, fenbuconazole, flusilazole, flutriafol, hexaconazole, imibenconazole, ipconazole, metconazole, myclobutanil, penconazole, propiconazole, prothioconazole, simeconazole, triadimefon, triadimenol, tebuconazole, tetraconazole, triticonazole, prochloraz, pefurazoate, imazalil, triflumizole, cyazofamid, benomyl, carbendazim, thia-bendazole, fuberidazole, ethaboxam, etridiazole and hymexazole, azaconazole, diniconazole-M, oxpoconazol, paclobutrazol, uniconazol, 1-(4-chloro-phenyl)-2-([1,2,4]triazol-1-yl)-cycloheptanol and imazalilsulfphate; B2) strobilurins, including azoxystrobin, dimoxystrobin, enestroburin, fluoxastrobin, kresoxim-methyl, metominostrobin, orysastrobin, picoxystrobin, pyraclostrobin, trifloxystrobin, enestroburin, methyl (2-chloro-5-[1-(3-methylbenzyloxyimino)ethyl]benzyl)carbamate, methyl(2-chloro-5-[1-(6-methylpyridin-2-ylmethoxyimino)ethyl]benzyl)carbamate and methyl 2-(ortho-(2,5-dimethylphenyloxymethylene)-phenyl)-3-methoxyacrylate, 2-(2-(6-(3-chloro-2-methyl-phenoxy)-5-fluoro-pyrimidin-4-yloxy)-phenyl)-2-methoxyimino-N-methyl-acetamide and 3-methoxy-2-(2-(N-(4-methoxy-phenyl)-cyclopropanecarboximidoylsulfanylmethyl)-phenyl)-acrylic acid methyl ester; B3) carboxamides, including carboxin, benalaxyl, benalaxyl-M, fenhexamid, flutolanil, furametpyr, mepronil, metalaxyl, mefenoxam, ofurace, oxadixyl, oxycarboxin, penthiopyrad, isopyrazam, thifluzamide, tiadinil, 3,4-dichloro-N-(2-cyanophenyl)isothiazole-5-carboxamide, dimethomorph, flumorph, flumetover, fluopicolide (picobenzamid), zoxamide, carpropamid, diclocymet, mandipropamid, N-(2-(4-[3-(4-chlorophenyl)prop-2-ynyloxy]-3-methoxyphenyl)ethyl)-2-methanesulfonyl-amino-3-methylbutyramide, N-(2-(4-[3-(4-chloro-phenyl)prop-2-ynyloxy]-3-methoxy-phenyl)ethyl)-2-ethanesulfonylamino-3-methylbutyramide, methyl 3-(4-chlorophenyl)-3-(2-isopropoxycarbonyl-amino-3-methyl-butyrylamino)propionate, N-(4′-bromobiphenyl-2-yl)-4-difluoromethyl̂-methylthiazole-δ-carboxamide, N-(4′-trifluoromethyl-biphenyl-2-yl)-4-difluoromethyl-2-methylthiazole-5-carboxamide, N-(4′-chloro-3′-fluorobiphenyl-2-yl)-4-difluoromethyl-2-methyl-thiazole-5-carboxamide, N-(3\4′-dichloro-4-fluorobiphenyl-2-yl)-3-difluoro-methyl-1-methyl-pyrazole-4-carboxamide, N-(3′,4′-dichloro-5-fluorobiphenyl-2-yl)-3-difluoromethyl-1-methylpyrazole-4-carboxamide, N-(2-cyano-phenyl)-3,4-dichloroisothiazole-5-carboxamide, 2-amino-4-methyl-thiazole-5-carboxanilide, 2-chloro-N-(1,1,3-trimethyl-indan-4-yl)-nicotinamide, N-(2-(1,3-dimethylbutyl)-phenyl)-1,3-dimethyl-5-fluoro-1H-pyrazole-4-carboxamide, N-(4′-chloro-3′,5-difluoro-biphenyl-2-yl)-3-difluoromethyl-1-methyl-1H-pyrazole-4-carboxamide, N-(4′-chloro-3′,5-difluoro-biphenyl-2-yl)-3-trifluoromethyl-1-methyl-1H-pyrazole-4-carboxamide, N-(3′,4′-dichloro-5-fluoro-biphenyl-2-yl)-3-trifluoromethyl-1-methyl-1H-pyrazole-4-carboxamide, N-(3′,5-difluoro-4′-methyl-biphenyl-2-yl)-3-difluoromethyl-1-methyl-1H-pyrazole-4-carboxamide, N-(3′,5-difluoro-4′-methyl-biphenyl-2-yl)-3-trifluoromethyl-1-methyl-1H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoro-methyl-1-methyl-1H-pyrazole-4-carboxamide, fluopyram, N-(3-ethyl-3,5-5-trimethyl-cyclohexyl)-3-formylamino-2-hydroxy-benzamide, oxytetracyclin, silthiofam, N-(6-methoxy-pyridin-3-yl) cyclopropanecarboxamide, 2-iodo-N-phenyl-benzamide, N-(2-bicyclo-propyl-2-yl-phenyl)-3-difluormethyl-1-methylpyrazol-4-ylcarboxamide, N-(3′,4′,5′-trifluorobiphenyl-2-yl)-1,3-dimethylpyrazol-4-ylcarboxamide, N-(3′,4′,5′-trifluorobiphenyl-2-yl)-1,3-dimethyl-5-fluoropyrazol-4-yl-carboxamide, N-(3′,4′,5′-trifluorobiphenyl-2-yl)-5-chloro-1,3-dimethyl-pyrazol-4-ylcarboxamide, N-(3′,4′,5′-trifluorobiphenyl-2-yl)-3-fluoromethyl-1-methylpyrazol-4-ylcarboxamide, N-(3′,4′,5′-trifluorobiphenyl-2-yl)-3-(chlorofluoromethyl)-1-methylpyrazol-4-ylcarboxamide, N-(3′,4′,5′-trifluorobiphenyl-2-yl)-3-difluoromethyl-1-methylpyrazol-4-ylcarboxamide, N-(3′,4′,5′-trifluorobiphenyl-2-yl)-3-difluoromethyl-5-fluoro-1-methylpyrazol-4-ylcarboxamide, N-(3′,4′,5′-trifluorobiphenyl-2-yl)-5-chloro-3-difluoromethyl-1-methylpyrazol-4-ylcarboxamide, N-(3′,4′,5′-trifluorobiphenyl-2-yl)-3-(chlorodifluoromethyl)-1-methylpyrazol-4-ylcarboxamide, N-(3′,4′,5′-trifluorobiphenyl-2-yl)-1-methyl-3-trifluoromethylpyrazol-4-ylcarboxamide, N-(3′,4′,5′-trifluorobiphenyl-2-yl)-5-fluoro-1-methyl-3-trifluoromethylpyrazol-4-ylcarboxamide, N-(3′,4′,5′-trifluorobiphenyl-2-yl)-5-chloro-1-methyl-3-trifluoromethylpyrazol-4-ylcarboxamide, N-(2′,4′,5′-trifluorobiphenyl-2-yl)-1,3-dimethylpyrazol-4-ylcarboxamide, N-(2′,4′,5′-trifluorobiphenyl-2-yl)-1,3-dimethyl-5-fluoropyrazol-4-ylcarboxamide, N-(2′,4′,5′-trifluorobiphenyl-2-yl)-5-chloro-1,3-dimethylpyrazol-4-ylcarboxamide, N-(2′,4′,5′-trifluorobiphenyl-2-yl)-3-fluoromethyl-1-methylpyrazol-4-ylcarboxamide, N-(2′,4′,5′-trifluorobiphenyl-2-yl)-3-(chlorofluoromethyl)-1-methylpyrazol-4-ylcarboxamide, N-(2′,4′,5′-trifluorobiphenyl-2-yl)-3-difluoromethyl-1-methylpyrazol-4-ylcarboxamide, N-(2′,4′,5′-trifluorobiphenyl-2-yl)-3-difluoromethyl-5-fluoro-1-methylpyrazol-4-ylcarboxamide, N-(2′,4′,5′-trifluorobiphenyl-2-yl)-5-chloro-3-difluoromethyl-1-methylpyrazol-4-ylcarboxamide, N-(2′,4′,5′-trifluorobiphenyl-2-yl)-3-(chlorodifluoromethyl)-1-methylpyrazol-4-ylcarboxamide, N-(2′,4′,5′-trifluorobiphenyl-2-yl)-1-methyl-3-trifluoromethylpyrazol-4-ylcarboxamide, N-(2′,4′,5′-trifluorobiphenyl-2-yl)-5-fluoro-1-methyl-3-trifluoromethylpyrazol-4-ylcarboxamide, N-(2′,4′,5′-trifluorobiphenyl-2-yl)-5-chloro-1-methyl-3-trifluoromethylpyrazol-4-ylcarboxamide, N-(3′,4′-dichloro-3-fluorobiphenyl-2-yl)-1-methyl-3-trifluoromethyl-1H-pyrazole-4-carboxamide, N-(3′,4′-dichloro-3-fluorobiphenyl-2-yl)-1-methyl-3-difluoromethyl-1H-pyrazole-4-carboxamide, N-(3′,4′-difluoro-3-fluorobiphenyl-2-yl)-1-methyl-3-trifluoromethyl-1H-pyrazole-4-carboxamide, N-(3′,4′-difluoro-3-fluorobiphenyl-2-yl)-1-methyl-S-difluoromethyl-1H-pyrazole-4-carboxamide, N-(3′-chloro-4′-fluoro-3-fluorobiphenyl-2-yl)-1-methyl-3-difluoromethyl-1H-pyrazole-4-carboxamide, N-(3′,4′-dichloro-4-fluorobiphenyl-2-yl)-1-methyl-3-trifluoromethyl-1H-pyrazole-4-carboxamide, N-(3′,4′-difluoro-4-fluorobiphenyl-2-yl)-1-methyl-S-trifluoromethyl-1H-pyrazole-4-carboxamide, N-(3′,4′-dichloro-4-fluorobiphenyl-2-yl)-1-methyl-3-difluoromethyl-1H-pyrazole-4-carboxamide, N-(3′,4′-difluoro-4-fluorobiphenyl-2-yl)-1-methyl-3-difluoromethyl-1H-pyrazole-4-carboxamide, N-(3′-chloro-4′-fluoro-4-fluorobiphenyl-2-yl)-1-methyl-S-difluoromethyl-1H-pyrazole-4-carboxamide, N-(3′,4′-dichloro-5-fluorobiphenyl-2-yl)-1-methyl-3-trifluoromethyl-1H-pyrazole-4-carboxamide, N-(3′,4′-difluoro-5-fluorobiphenyl-2-yl)-1-methyl-3-trifluoromethyl-1H-pyrazole-4-carboxamide, N-(3′,4′-dichloro-5-fluorobiphenyl-2-yl)-1-methyl-S-difluoromethyl-1H-pyrazole-carboxamide, N-(3′,4′-difluoro-5-fluorobiphenyl-2-yl)-1-methyl-3-difluoromethyl-1H-pyrazole-4-carboxamide, N-(3′,4′-dichloro-5-fluorobiphenyl-2-yl)-1,3-dimethyl-1H-pyrazole-4-carboxamide, N-(3′-chloro-4′-fluoro-5-fluorobiphenyl-2-yl)-1-methyl-3-difluoromethyl-1H-pyrazole-4-carboxamide, N-(4′-fluoro-4-fluorobiphenyl-2-yl)-1-methyl-3-trifluoromethyl-1H-pyrazole-4-carboxamide, N-(4′-fluoro-5-fluorobiphenyl-2-yl)-1-methyl-3-trifluoromethyl-1H-pyrazole-4-carboxamide, N-(4′-chloro-5-fluorobiphenyl-2-yl)-1-methyl-3-trifluoromethyl-1H-pyrazole-4-carboxamide, N-(4′-methyl-5-fluorobiphenyl-2-yl)-1-methyl-3-trifluoromethyl-1H-pyrazole-4-carboxamide, N-(4′-fluoro-5-fluorobiphenyl-2-yl)-1,3-dimethyl-1H-pyrazole-4-carboxamide, N-(4′-chloro-5-fluorobiphenyl-2-yl)-1,3-dimethyl-1H-pyrazole-4-carboxamide, N-(4′-methyl-5-fluorobiphenyl-2-yl)-1,3-dimethyl-1H-pyrazole-4-carboxamide, N-(4′-fluoro-6-fluorobiphenyl-2-yl)-1-methyl-3-trifluoromethyl-1H-pyrazole-4-carboxamide, N-(4′-chloro-6-fluorobiphenyl-2-yl)-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[4′-(trifluoromethylthio)-biphenyl-2-yl]-3-difluoromethyl-1-methyl-1H-pyrazole-4-carboxamide and N-[4′-(trifluoromethylthio)-biphenyl-2-yl]-1-methyl-3-trifluoromethyl-1-methyl-1H-pyrazole-4-carboxamide; B4) heterocyclic compounds, including fluazinam, pyrifenox, bupirimate, cyprodinil, fenarimol, ferimzone, mepanipyrim, nuarimol, pyrimethanil, triforine, fenpiclonil, fludioxonil, aldimorph, dodemorph, fenpropimorph, tridemorph, fenpropidin, iprodione, procymidone, vinclozolin, famoxadone, fenamidone, octhilinone, proben-azole, 5-chloro-7-(4-methyl-piperidin-1-yl)-6-(2,4,6-trifluorophenyl)-[1,2,4]triazolo[1,5-a]pyrimidine, anilazine, diclomezine, pyroquilon, proquinazid, tricyclazole, 2-butoxy-6-iodo-3-propylchromen-4-one, acibenzolar-S-methyl, captafol, captan, dazomet, folpet, fenoxanil, quinoxyfen, N,N-dimethyl-3-(3-bromo-6-fluoro-2-methylindole-1-sulfonyl)-[1,2,4]triazole-1-sulfonamide, 5-ethyl-6-octyl-[1,2,4]triazolo[1,5-a]pyrimidin-2,7-diamine, 2,3,5,6-tetrachloro-4-methanesulfonyl-pyridine, 3,4,5-trichloro-pyridine-2,6-di-carbonitrile, N-(1-(5-bromo-3-chloro-pyridin-2-yl)-ethyl)-2,4-dichloro-nicotinamide, N-((5-bromo-3-chloro pyridin-2-yl)-methyl)-2,4-dichloro-nicotinamide, diflumetorim, nitrapyrin, dodemorphacetate, fluoroimid, blasticidin-S, chinomethionat, debacarb, difenzoquat, difenzoquat-methylsulphat, oxolinic acid and piperalin; B5) carbamates, including mancozeb, maneb, metam, methasulphocarb, metiram, ferbam, propineb, thiram, zineb, ziram, diethofencarb, iprovalicarb, benthiavalicarb, propamocarb, propamocarb hydrochlorid, 4-fluorophenyl N-(1-(1-(4-cyanophenyl)-ethanesulfonyl)but-2-yl)carbamate, methyl 3-(4-chloro-phenyl)-3-(2-isopropoxycarbonylamino-3-methyl-butyrylamino)propanoate; or B6) other fungicides, including guanidine, dodine, dodine free base, iminoctadine, guazatine, antibiotics: kasugamycin, oxytetracyclin and its salts, streptomycin, polyoxin, validamycin A, nitrophenyl derivatives: binapacryl, dinocap, dinobuton, sulfur-containing heterocyclyl compounds: dithianon, isoprothiolane, organometallic compounds: fentin salts, organophosphorus compounds: edifenphos, iprobenfos, fosetyl, fosetyl-aluminum, phosphorous acid and its salts, pyrazophos, tolclofos-methyl, organochlorine compounds: dichlofluanid, flusulfamide, hexachloro-benzene, phthalide, pencycuron, quintozene, thiophanate, thiophanate-methyl, tolylfluanid, others: cyflufenamid, cymoxanil, dimethirimol, ethirimol, furalaxyl, metrafenone and spiroxamine, guazatine-acetate, iminoc-tadine-triacetate, iminoctadine-tris(albesilate), kasugamycin hydrochloride hydrate, dichlorophen, pentachlorophenol and its salts, N-(4-chloro-2-nitro-phenyl)-N-ethyl-4-methyl-benzenesulfonamide, dicloran, nitrothal-isopropyl, tecnazen, biphenyl, bronopol, diphenylamine, mildiomycin, oxincopper, prohexadione calcium, N-(cyclopropylmethoxyimino-(6-difluoromethoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluormethyl-4-(3-trimethylsilanyl-propoxy)-phenyl)-N-ethyl-N-methylformamidine and N′-(5-difluormethyl-2-methyl-4-(3-trimethylsilanyl-propoxy)-phenyl)-N-ethyl-N-methyl formamidine.

Herbicides: C1) acetyl-CoA carboxylase inhibitors (ACC), for example cyclohexenone oxime ethers, such as alloxydim, clethodim, cloproxydim, cycloxydim, sethoxydim, tralkoxydim, butroxydim, clefoxydim or tepraloxydim; phenoxyphenoxypropionic esters, such as clodinafop-propargyl, cyhalofop-butyl, diclofop-methyl, fenoxaprop-ethyl, fenoxaprop-P-ethyl, fenthiapropethyl, fluazifop-butyl, fluazifop-P-butyl, haloxyfop-ethoxyethyl, haloxyfop-methyl, haloxyfop-P-methyl, isoxapyrifop, propaquizafop, quizalofop-ethyl, quizalofop-P-ethyl or quizalofop-tefuryl; or arylaminopropionic acids, such as flamprop-methyl or flamprop-isopropyl; C2 acetolactate synthase inhibitors (ALS), for example imidazolinones, such as imazapyr, imazaquin, imazamethabenz-methyl(imazame), imazamox, imazapic or imazethapyr; pyrimidyl ethers, such as pyrithiobac-acid, pyrithiobac-sodium, bispyribac-sodium. KIH-6127 or pyribenzoxym; sulfonamides, such as florasulam, flumetsulam or metosulam; or sulfonylureas, such as amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, halosulfuron-methyl, imazosulfuron, metsulfuron-methyl, nicosulfuron, primisulfuron-methyl, prosulfuron, pyrazosulfuron-ethyl, rimsulfuron, sulfometuron-methyl, thifensulfuron-methyl, triasulfuron, tribenuron-methyl, triflusulfuron-methyl, tritosulfuron, sulfosulfuron, foramsulfuron or iodosulfuron; C3) amides, for example allidochlor (CDAA), benzoylprop-ethyl, bromobutide, chiorthiamid. diphenamid, etobenzanidibenzchlomet), fluthiamide, fosamin or monalide; C4) auxin herbicides, for example pyridinecarboxylic acids, such as clopyralid or picloram; or 2,4-D or benazolin; C5) auxin transport inhibitors, for example naptalame or diflufenzopyr; C6) carotenoid biosynthesis inhibitors, for example benzofenap, clomazone (dimethazone), diflufenican, fluorochloridone, fluridone, pyrazolynate, pyrazoxyfen, isoxaflutole, isoxachlortole, mesotrione, sulcotrione (chlormesulone), ketospiradox, flurtamone, norflurazon or amitrol; C7) enolpyruvylshikimate-3-phosphate synthase inhibitors (EPSPS), for example glyphosate or sulfosate; C8) glutamine synthetase inhibitors, for example bilanafos (bialaphos) or glufosinate-ammonium; C9) lipid biosynthesis inhibitors, for example anilides, such as anilofos or mefenacet; chloroacetanilides, such as dimethenamid, S-dimethenamid, acetochlor, alachlor, butachlor, butenachlor, diethatyl-ethyl, dimethachlor, metazachlor, metolachlor, S-metolachlor, pretilachlor, propachlor, prynachlor, terbuchlor, thenylchlor or xylachlor; thioureas, such as butylate, cycloate, di-allate, dimepiperate, EPTC. esprocarb, molinate, pebulate, prosulfocarb, thiobencarb (benthiocarb), tri-allate or vemolate; or benfuresate or perfluidone; C10) mitosis inhibitors, for example carbamates, such as asulam, carbetamid, chlorpropham, orbencarb, pronamid (propyzamid), propham or tiocarbazil; dinitroanilines, such as benefin, butralin, dinitramin, ethalfluralin, fluchloralin, oryzalin, pendimethalin, prodiamine or trifluralin; pyridines, such as dithiopyr or thiazopyr; or butamifos, chlorthal-dimethyl (DCPA) or maleic hydrazide; C11) protoporphyrinogen IX oxidase inhibitors, for example diphenyl ethers, such as acifluorfen, acifluorfen-sodium, aclonifen, bifenox, chlomitrofen (CNP), ethoxyfen, fluorodifen, fluoroglycofen-ethyl, fomesafen, furyloxyfen, lactofen, nitrofen, nitrofluorfen or oxyfluorfen; oxadiazoles, such as oxadiargyl or oxadiazon; cyclic imides, such as azafenidin, butafenacil, carfentrazone-ethyl, cinidon-ethyl, flumiclorac-pentyl, flumioxazin, flumipropyn, flupropacil, fluthiacet-methyl, sulfentrazone or thidiazimin; or pyrazoles, such as ET-751.JV 485 or nipyraclofen; C12) photosynthesis inhibitors, for example propanil, pyridate or pyridafol; benzothiadiazinones, such as bentazone; dinitrophenols, for example bromofenoxim, dinoseb, dinoseb-acetate, dinoterb or DNOC; dipyridylenes, such as cyperquat-chloride, difenzoquat-methylsulfate, diquat or paraquat-dichloride; ureas, such as chlorbromuron, chlorotoluron, difenoxuron, dimefuron, diuron, ethidimuron, fenuron, fluometuron, isoproturon, isouron, linuron, methabenzthiazuron, methazole, metobenzuron, metoxuron, monolinuron, neburon, siduron or tebuthiuron; phenols, such as bromoxynil or ioxynil; chloridazon; triazines, such as ametryn, atrazine, cyanazine, desmein, dimethamethryn, hexazinone, prometon, prometryn, propazine, simazine, simetryn, terbumeton, terbutryn, terbutylazine or trietazine; triazinones, such as metamitron or metribuzin; uracils, such as bromacil, lenacil or terbacil; or biscarbamates, such as desmedipham or phenmedipham; C13) synergists, for example oxiranes, such as tridiphane; C14) CIS cell wall synthesis inhibitors, for example isoxaben or dichlobenil; C15) various other herbicides, for example dichloropropionic acids, such as dalapon; dihydrobenzofurans, such as ethofumesate; phenylacetic acids, such as chlorfenac (fenac); or aziprotryn, barban, bensulide, benzthiazuron, benzofluor, buminafos, buthidazole, buturon, cafenstrole, chlorbufam, chlorfenprop-methyl, chloroxuron, cinmethylin, cumyluron, cycluron, cyprazine, cyprazole, dibenzyluron, dipropetryn, dymron, eglinazin-ethyl, endothall, ethiozin, flucabazone, fluorbentranil, flupoxam, isocarbamid, isopropalin, karbutilate, mefluidide, monuron, napropamide, napropanilide, nitralin, oxaciclomefone, phenisopham, piperophos, procyazine, profluralin, pyributicarb, secbumeton, sulfallate (CDEC), terbucarb, triaziflam, triazofenamid or trimeturon; or their environmentally compatible salts.

Nematicides or bionematicides: Benomyl, cloethocarb, aldoxycarb, tirpate, diamidafos, fenamiphos, cadusafos, dichlofenthion, ethoprophos, fensulfothion, fosthiazate, heterophos, isamidofof, isazofos, phosphocarb, thionazin, imicyafos, mecarphon, acetoprole, benclothiaz, chloropicrin, dazomet, fluensulfone, 1,3-dichloropropene(telone), dimethyl disulfide, metam sodium, metam potassium, metam salt (all MITC generators), methyl bromide, biological soil amendments (e.g., mustard seeds, mustard seed extracts), steam fumigation of soil, allyl isothiocyanate (AITC), dimethyl sulfate, furfual (aldehyde).

Suitable plant growth regulators of the present invention include the following: Plant Growth Regulators: D1) Antiauxins, such as clofibric acid, 2,3,5-tri-iodobenzoic acid; D2) Auxins such as 4-CPA, 2,4-D, 2,4-DB, 2,4-DEP, dichlorprop, fenoprop, IAA, IBA, naphthaleneacetamide, α-naphthaleneacetic acids, 1-naphthol, naphthoxyacetic acids, potassium naphthenate, sodium naphthenate, 2,4,5-T; D3) cytokinins, such as 2iP, benzyladenine, 4-hydroxyphenethyl alcohol, kinetin, zeatin; D4) defoliants, such as calcium cyanamide, dimethipin, endothal, ethephon, merphos, metoxuron, pentachlorophenol, thidiazuron, tribufos; D5) ethylene inhibitors, such as aviglycine, 1-methylcyclopropene; D6) ethylene releasers, such as ACC, etacelasil,ethephon, glyoxime; D7) gametocides, such as fenridazon, maleic hydrazide; D8) gibberellins, such as gibberellins, gibberellic acid; D9) growth inhibitors, such as abscisic acid, ancymidol, butralin, carbaryl, chlorphonium, chlorpropham, dikegulac, flumetralin, fluoridamid, fosamine, glyphosine, isopyrimol, jasmonic acid, maleic hydrazide, mepiquat, piproctanyl, prohydrojasmon, propham, tiaojiean, 2,3,5-tri-iodobenzoic acid; D10) morphactins, such as chlorfluren, chlorflurenol, dichlorflurenol, flurenol; D11) growth retardants, such as chlormequat, daminozide, flurprimidol, mefluidide, paclobutrazol, tetcyclacis, uniconazole; D12) growth stimulators, such as brassinolide, brassinolide-ethyl, DCPTA, forchlorfenuron, hymexazol, prosuler, triacontanol; D13) unclassified plant growth regulators, such as bachmedesh, benzofluor, buminafos, carvone, choline chloride, ciobutide, clofencet, cyanamide, cyclanilide, cycloheximide, cyprosulfamide, epocholeone, ethychlozate, ethylene, fuphenthiourea, furalane, heptopargil, holosulf, inabenfide, karetazan, lead arsenate, methasulfocarb, prohexadione, pydanon, sintofen, triapenthenol, trinexapac.

The fertilizer can be a liquid fertilizer. The term “liquid fertilizer” refers to a fertilizer in a fluid or liquid form containing various ratios of nitrogen, phosphorous and potassium (for example, but not limited to, 10% nitrogen, 34% phosphorous and 0% potassium) and micronutrients, commonly known as starter fertilizers that are high in phosphorus and promote rapid and vigorous root growth.

Chemical formulations of the present invention can be in any appropriate conventional form, for example an emulsion concentrate (EC), a suspension concentrate (SC), a suspo-emulsion (SE), a capsule suspension (CS), a water dispersible granule (WG), an emulsifiable granule (EG), a water in oil emulsion (EO), an oil in water emulsion (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a dispersible concentrate (DC), a wettable powder (WP) or any technically feasible formulation in combination with agriculturally acceptable adjuvants.

In another embodiment of the present invention, a plant seed is provided that is coated with a composition including spores of a biologically pure culture of Bacillus amyloliquefaciens RTI301 deposited as ATCC No. PTA-121165, or a mutant thereof having all the identifying characteristics thereof, present in an amount suitable to benefit plant growth and/or to confer protection against a pathogenic infection in a susceptible plant. The growth benefit of the plant and/or the conferred protection can be exhibited by improved seedling vigor, improved root development, improved plant growth, improved plant health, increased yield, improved appearance, improved resistance to plant pathogens, reduced pathogenic infection, or a combination thereof.

The composition coated on the plant seed can include an amount of Bacillus amyloliquefaciens spores from about 1.0×10² CFU/seed to about 1.0×10⁹ CFU/seed.

The plant seed can include, but is not limited to, the seed of monocots, dicots, Cereals, Corn, Sweet Corn, Popcorn, Seed Corn, Silage Corn, Field Corn, Rice, Wheat, Barley, Sorghum, Brassica Vegetables, Broccoli, Cabbage, Cauliflower, Brussels Sprouts, Collards, Kale, Mustard Greens, Kohlrabi, Bulb Vegetables, Onion, Garlic, Shallots, Fruiting Vegetables, Pepper, Tomato, Eggplant, Ground Cherry, Tomatillo, Okra, Grape, Herbs/ Spices, Cucurbit Vegetables, Cucumber, Cantaloupe, Melon, Muskmelon, Squash, Watermelon, Pumpkin, Eggplant, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Cotton, Flax, Peanut, Rapeseed, Safflower, Sesame, Sunflower, Soybean, Root/Tuber and Corm Vegetables, Carrot, Potato, Sweet Potato, Beets, Ginger, Horseradish, Radish, Ginseng, Turnip, sugarcane, sugarbeet, Grass, or Turf grass.

In one or more embodiments, the plant seed can include seed of a drybean, a corn, a wheat, a soybean, a canola, a rice, a cucumber, a pepper, a tomato, a squash, a cotton, a grass, and a turf grass.

The pathogenic infection treated by the coated plant seed can be caused by a plant pathogen including, for example, but not limited to a plant fungal pathogen, a plant bacterial pathogen, a Botrytis spp., a Botrytis cinerea, a Botrytis squamosa, an Erwinia spp., an Erwinia carotovora, an Erwinia amylovora, a Fusarium spp., a Fusarium colmorum, a Fusarium graminearum, a Fusarium oxysporum, a Fusarium oxysporum f. sp. Cubense, a Fusarium oxysporum f. sp. Lycopersici, a Fusarium virguliforme, a Xanthomonas spp., a Xanthomonas axonopodis, a Xanthomonas campestris pv. carotae, a Xanthomonas pruni, a Xanthomonas arboricola, a Xanthomonas oryzae pv. oryzae, a Pseudomonas spp., a Pseudomonas syringae pv. Tomato, a Phytophthora spp., a Phytophthora infestans, a Phytophthora parasitica, a Phytophthora sojae, a Phytophthora capsici, a Phytophthora cinnamon, a Phytophthora fragariae, a Phytophthora spp., a Phytophthora ramorum, a Phytophthora palmivara, a Phytophthora nicotianae, a Rhizoctonia spp., a Rhizoctonia solani, a Rhizoctonia zeae, a Rhizoctonia oryzae, a Rhizoctonia caritae, a Rhizoctonia cerealis, a Rhizoctonia crocorum, a Rhizoctonia fragariae, a Rhizoctonia ramicola, a Rhizoctonia rubi, a Rhizoctonia leguminicola, a Macrophomina phaseolina, a Magnaorthe oryzae, a Pythium spp., a Pythium ultimum, a Pythium aphanidermatum, a Pythium irregularum, a Pythium ulosum, a Pythium lutriarium, a Pythium sylvatium, a Ustilago spp., a Ustilago nuda, a Ustilago maydis, a Ustilago scitaminea, a Claviceps spp., a Claviceps puprrea, a Tilletia spp., a Tilletia tritici, a Tilletia laevis, a Tilletia horrid, a Tilletia controversa, a Phoma spp., a Phoma glycinicola, a Phoma exigua, a Phoma lingam, a Cocliobolus sativus, a Gaeumanomyces gaminis, a Colleototricum spp., or combinations thereof.

The composition coated onto the plant seed can further include one or a combination of a microbial, a biological, or a chemical insecticide, fungicide, nematicide, bacteriocide, or plant growth regulator present in an amount suitable to benefit plant growth and/or to confer protection against a pathogenic infection in a susceptible plant. The insecticide can include bifenthrin. The nematicide can include cadusafos. The insecticide can include bifenthrin and clothianidin.

In another embodiment of the present invention, a method is provided for one or both of benefiting growth of a plant or conferring protection against pathogenic infection in a susceptible plant, the method including planting a seed of the plant or regenerating a vegetative cutting/tissue of the plant in a suitable growth medium, wherein the seed has been coated or the vegetative cutting/tissue has been inoculated with a composition comprising a biologically pure culture of Bacillus amyloliquefaciens RTI301 deposited as ATCC PTA-121165, or a mutant thereof having all the identifying characteristics thereof, wherein growth of the plant from the seed or the vegetative cutting/tissue is benefited and/or protection against pathogenic infection is conferred. The growth benefit of the plant and/or the conferred protection can be exhibited by improved seedling vigor, improved root development, improved plant growth, improved plant health, increased yield, improved appearance, improved resistance to plant pathogens, reduced pathogenic infection, or a combination thereof.

In one embodiment, the method can further include applying a liquid fertilizer to: soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In one embodiment, a method is provided for benefiting plant growth by conferring protection against or reducing pathogenic infection in a susceptible plant while minimizing the build-up of resistance against the treatment. The method includes delivering to the susceptible plant in separate applications and in altering time intervals a first composition and a second composition, wherein each of the first and second compositions are delivered in an amount suitable to to confer protection against or reduce pathogenic infection in the plant. The first composition includes a biologically pure culture of Bacillus amyloliquefaciens RTI301 deposited as ATCC No. PTA-121165, or a mutant thereof having all the identifying characteristics thereof. The second composition includes one or more chemical active agents having fungicidal or a bacteriocidal properties. In the method the first and second compositions are delivered in the altering time intervals to one or a combination of foliage of the plant, bark of the plant, fruit of the plant, flowers of the plant, seed of the plant, roots of the plant, a cutting of the plant, a graft of the plant, callus tissue of the plant, or soil or growth medium surrounding the plant. In the method, the total amount of the chemical active agent(s) required to confer protection against and/or reduce the pathogenic infection is decreased and the build-up of resistance against the treatment is minimized. The growth benefit of the plant and/or the conferred protection can be exhibited by improved seedling vigor, improved root development, improved plant growth, improved plant health, increased yield, improved appearance, improved resistance to plant pathogens, reduced pathogenic infection, or a combination thereof.

The first composition can further include one or a combination of a carrier, a surfactant, a dispersant, or a yeast extract. The yeast extract can be delivered at a rate for benefiting plant growth ranging from about 0.01% to 0.2% w/w.

In the method for benefiting plant growth by conferring protection against or reducing pathogenic infection in a susceptible plant while minimizing the build-up of resistance against the treatment, the altering time intervals can range from 1 day to 10 days apart and can be 5 to 7 days apart. The timing of the first application can depend on the particular crop and can range from at the time of planting, a fews weeks after crop emergence, at the time of flowering, upon disease emergence, or prior to expectation of disease emergence. Each of the first and the second compositions can be delivered to the foliage of the plant, the fruit of the plant, or the flowers of the plant. The amount delivered that is suitable to confer protection against or reduce pathogenic infection in the plant can be from about 1.0×10¹⁰ CFU/ha to about 1.0×10¹⁴ CFU/ha Bacillus amyloliquefaciens RTI301. The amount delivered that is suitable to confer protection against or reduce pathogenic infection in the plant can be from about 1.0×10¹⁰ CFU/ha to about 1.0×10¹⁴ CFU/ha Bacillus amyloliquefaciens RTI301 and about 0.01% to 0.2% w/w yeast extract.

The one or more chemical active agents for delivering to the susceptible plant in separate applications and in altering time intervals can include, for example, but are not limited to one or a combination of strobilurine, a triazole, flutriafol, tebuconazole, prothiaconazole, expoxyconazole, fluopyram, chlorothalonil, thiophanate-methyl, Copper Hydroxide fungicide, an EDBC-based fungicide, mancozeb, a succinase dehydrogenase (SDHI) fungicide, bixafen, iprodione, dimethomorph, or valifenalate.

The one or more chemical active agents for delivering to the susceptible plant in separate applications and in altering time intervals can include Fluopyram plus Tebuconazole and delivery of the first composition comprising the RTI301 can replace the delivery of the Chlorothalonil fungicide. The plant can be a cucurbit and the pathogenic infection can be caused by Powdery mildew.

The one or more chemical active agents for delivering to the susceptible plant in separate applications and in altering time intervals can include Thiophanate-methyl fungicide and delivery of the first composition comprising the RTI301 can replace the delivery of a Prothioconazole fungicide.

The one or more chemical active agents for delivering to the susceptible plant in separate applications and in altering time intervals can include copper hydroxide fungicide and delivery of the first composition comprising the RTI301 can replace the delivery of a chlorothalonil fungicide.

In one embodiment, a product is provided comprising: a first composition comprising a biologically pure culture of Bacillus amyloliquefaciens RTI301 deposited as ATCC No. PTA-121165, or a mutant thereof having all the identifying characteristics thereof; a second composition comprising one or a combination of a microbial, a biological, or a chemical insecticide, fungicide, nematicide, bacteriocide, herbicide, plant extract, plant growth regulator, or fertilizer, wherein the first and second compositions are separately packaged, and wherein each composition is in an amount suitable for one or both of benefiting plant growth or conferring protection against a pathogenic infection in a susceptible plant; and instructions for delivering in an amount suitable to benefit plant growth, a combination of the first and second compositions to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

The insecticide in the product can be one or a combination of pyrethroids, bifenthrin, tefluthrin, zeta-cypermethrin, organophosphates, chlorethoxyphos, chlorpyrifos, tebupirimphos, cyfluthrin, fiproles, fipronil, nicotinoids, or clothianidin.

The first composition in the product can further include one or a combination of a carrier, a surfactant, a dispersant, or a yeast extract.

In the product, the first and second compositions can be in the form of a liquid, a dust, a spreadable granule, a dry wettable powder, or a dry wettable granule. In one embodiment, the first composition is in the form of a liquid and the Bacillus amyloliquefaciens RTI301 is present at a concentration of from about 1.0×10⁸ CFU/ml to about 1.0×10¹² CFU/ml. In one embodiment, the first composition is in the form of a dust, a dry wettable powder, a spreadable granule, or a dry wettable granule and the Bacillus amyloliquefaciens RTI301 is present in an amount of from about 1.0×10⁸ CFU/g to about 1.0×10¹² CFU/g. In one embodiment, the first composition is in the form of an oil dispersion and the Bacillus amyloliquefaciens RTI301 is present at a concentration of from about 1.0×10⁸ CFU/ml to about 1.0×10¹² CFU/ml.

In one embodiment of the present invention, a composition is provided, the composition including at least one of an isolated Fengycin MA compound, an isolated Fengycin MB compound, an isolated Fengycin MC compound, an isolated Dehydroxyfengycin MA compound, an isolated Dehydroxyfengycin MB compound, an isolated Dehydroxyfengycin MC compound, an isolated Fengycin H compound, an isolated Dehydroxyfengycin H compound, an isolated Fengycin I compound, and an isolated Dehydroxyfengycin I compound in an amount suitable to confer one or both of a growth benefit on the plant or protection against a pathogenic infection in a susceptible plant, the Fengycin and Dehydroxyfengycin compounds having the formula:

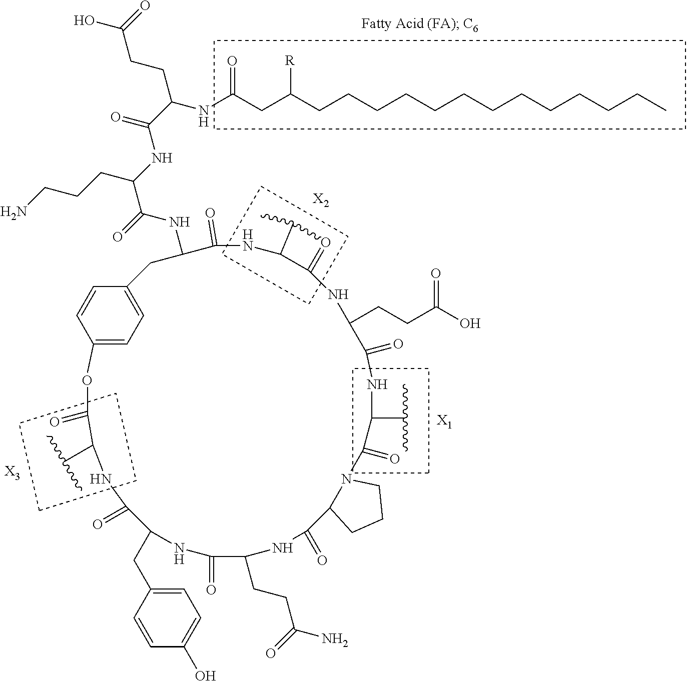

wherein R is OH, n ranges from 8 to 20, FA is linear, iso, or anteiso and: X₁ is Ala, X₂ is Thr, and X₃ is Met for Fengycin MA; X₁ is Val, X₂ is Thr, and X₃ is Met for Fengycin MB; X₁ is Aba, X₂ is Thr, and X₃ is Met for Fengycin MC; X₁ is Val, X₂ is Thr, and X₃ is Hcy for Fengycin H; and X₁ is Ile, X₂ is Thr, and X₃ is Ile for Fengycin I; and
wherein R is H, n ranges from 8 to 20, FA is linear, iso, or anteiso and: X₁ is Ala, X₂ is Thr, and X₃ is Met for Dehydroxyfengycin MA; X₁ is Val, X₂ is Thr, and X₃ is Met for Dehydroxyfengycin MB; X₁ is Aba, X₂ is Thr, and X₃ is Met for Dehydroxyfengycin MC; X₁ is Val, X₂ is Thr, and X₃ is Hcy for Dehydroxyfengycin H; and X₁ is Ile, X₂ is Thr, and X₃ is Ile for Dehydroxyfengycin I.

In another embodiment, the composition further comprises one or a combination of additional isolated Fengycin-and Dehydroxyfengycin-like compounds listed in Table XIII in an amount suitable to confer one or both of a growth benefit on the plant or protection against a pathogenic infection in the susceptible plant.

The growth benefit of the plant and/or the conferred protection can be exhibited by improved seedling vigor, improved root development, improved plant growth, improved plant health, increased yield, improved appearance, improved resistance to plant pathogens, reduced pathogenic infection, or a combination thereof.

The Fengycin-MA, -MB, -MC, -H, and -I compounds and the Dehydroxyfengycin-MA, -MB, -MC, -H, and -I compounds and one or a combination of additional Fengycin-and Dehydroxyfengycin-like compounds can be isolated by first culturing the RTI301 Bacillus amyloliquefaciens strain, or another Bacillus amyloliquefaciens strain that produces the Fengycin-MA, -MB, -MC, -H, and -I compounds and the Dehydroxyfengycin-MA, -MB, -MC, -H, and -I compounds, under suitable conditions well known to those of skill in the art, such as, for example, those conditions described in the EXAMPLES herein, including, but not limited to, culturing the strain for 3 to 6 days in 869 or M2 media. The Fengycin-like and Dehydroxyfengycin-like cyclic lipopeptides present in the Bacillus amyloliquefaciens culture supernatant can then be further isolated using methods well known to those of skill in the art. For example, the Bacillus amyloliquefaciens culture supernatant can be acidified to pH 2 as described herein at EXAMPLE 16 (, or treated with CaCl₂ (Ajesh, K et al., 2013, “Purification and characterization of antifungal lipopeptide from a soil isolated strain of Bacillus cereus.” In: Worldwide research efforts in the fighting against microbial pathogens: from basic research to technological developments. A. Mendez-Vilas (editor). pp: 227-231) or NH₄SO₄ (Kim, S H et al., 2000, Biotechnol Appl Biochem. 31 (Pt 3):249-253) with or without combining this with an organic extraction step (Kim, P I et al., 2004, J Appl Microbiol. 97(5): 942-949) such as various forms of phase separation including but not limited to direct liquid partitioning, membrane ultrafiltration, and foam fractionation (Baker, S C et al., 2010, Adv Exp Med Biol. 672:281-288).

In one embodiment, the Fengycin-MA, -MB, -MC, -H, and -I compounds and the Dehydroxyfengycin-MA, -MB, -MC, -H, and -I compounds and the one or a combination of additional Fengycin-and Dehydroxyfengycin-like compounds listed in Table XIII can be isolated from a biologically pure culture of a Bacillus amyloliquefaciens strain that can produce these compounds.

In one embodiment, the Fengycin-MA, -MB, -MC, -H, and -I compounds and the Dehydroxyfengycin-MA, -MB, -MC, -H, and -I compounds and the one or a combination of additional Fengycin-and Dehydroxyfengycin-like compounds listed in Table XIII can be isolated from a biologically pure culture of Bacillus amyloliquefaciens RTI301 deposited as ATCC No. PTA-121165.

In one embodiment, an extract is provided of a biologically pure culture of a Bacillus amyloliquefaciens strain, the extract including a Fengycin-MA, -MB, -MC, -H, and -I compound and a Dehydroxyfengycin-MA, -MB, -MC, -H, and -I compound and one or a combination of additional Fengycin-and Dehydroxyfengycin-like compounds listed in Table XIII.

In one embodiment, an extract is provided of a biologically pure culture of Bacillus amyloliquefaciens RTI301 deposited as ATCC No. PTA-121165, the extract including a Fengycin-MA, -MB, -MC, -H, and -I compound and a Dehydroxyfengycin-MA, -MB, -MC, -H, and -I compound and one or a combination of additional Fengycin-and Dehydroxyfengycin-like compounds listed in Table XIII.

The compositions including at least one of the Fengycin-MA, -MB, -MC, -H, and -I compounds and the Dehydroxyfengycin-MA, -MB, -MC, -H, and -I compounds and optionally the one or a combination of additional isolated Fengycin- and Dehydroxyfengycin-like compounds can further include one or a combination of a microbial, a biological, or a chemical insecticide, fungicide, nematicide, bacteriocide, herbicide, plant extract, plant growth regulator, or fertilizer, present in an amount suitable to benefit plant growth and/or to confer protection against pathogenic infection in the susceptible plant.

The fungicide can include an extract from Lupinus albus. The fungicide can include a BLAD polypeptide. The BLAD polypeptide can be a fragment of the naturally occurring seed storage protein from sweet lupine (Lupinus albus) that acts on susceptible fungal pathogens by causing damage to the fungal cell wall and disrupting the inner cell membrane. The fungicide can include about 20% of a BLAD polypeptide.

The compositions including the at least one of the Fengycin-MA, -MB, -MC, -H, and -I compounds and the Dehydroxyfengycin-MA, -MB, -MC, -H, and -I compounds can be in the form of a liquid, an oil dispersion, a dust, a spreadable granule, or a dry wettable granule.

In one embodiment, a method is provided for benefiting plant growth and/or conferring protection against a plant pathogenic infection that includes applying an effective amount of the extract or the composition comprising the isolated Fengycin-MA, -MB, -MC, -H, and -I compounds and the Dehydroxyfengycin-MA, -MB, -MC, -H, and -I compounds and one or a combination of additional isolated Fengycin-and Dehydroxyfengycin-like compounds to the plant or fruit, or to the roots or soil around the roots of the plants to benefit the plant growth and/or conferring protection against the plant pathogenic infection. The growth benefit of the plant and/or the conferred protection can be exhibited by improved seedling vigor, improved root development, improved plant growth, improved plant health, increased yield, improved appearance, improved resistance to plant pathogens, reduced pathogenic infection, or a combination thereof.

In the method for applying an effective amount of the extract or the composition comprising the isolated Fengycin-MA, -MB, -MC, -H, and -I compounds and the Dehydroxyfengycin-MA, -MB, -MC, -H, and -I compounds and one or a combination of additional isolated Fengycin-like or Dehydroxyfengycin-like compounds compounds, the plant can include, for example, monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hardwood cuttings, Chestnuts, Oak, Maple, sugarcane, or sugarbeet.

In the method for applying an effective amount of the extract or the composition comprising the isolated Fengycin-MA, -MB, -MC, -H, and -I compounds and the Dehydroxyfengycin-MA, -MB, -MC, -H, and -I compounds and one or a combination of additional isolated Fengycin- or Dehydroxyfengycin-like compounds, the pathogenic infection can be caused by a plant pathogen, including, for example, a plant fungal pathogen, a plant bacterial pathogen, a rust fungus a Botrytis spp., a Botrytis cinerea, a Botrytis squamosa, an Erwinia spp., an Erwinia carotovora, an Erwinia amylovora, a Dickeya spp., a Dickeya dadantii, a Dickeya solani, an Agrobacterium spp., a Agrobacterium tumefaciens, a Xanthomonas spp., a Xanthomonas axonopodis, a Xanthomonas campestris pv. carotae, a Xanthomonas pruni, a Xanthomonas arboricola, a Xanthomonas oryzae pv. oryzae, a Xylella spp., a Xylella fastidiosa, a Candidatus spp., a Candidatus liberibacter, a Fusarium spp., a Fusarium colmorum, a Fusarium graminearum, a Fusarium oxysporum, a Fusarium oxysporum f. sp. Cubense, a Fusarium oxysporum f. sp. Lycopersici, a Fusarium virguliforme, a Sclerotinia spp., a Sclerotinia sclerotiorum, a Sclerotinia minor, Sclerotinia homeocarpa, a Cercospora/Cercosporidium spp., an Uncinula spp., an Uncinula necator (Powdery Mildew), a Podosphaera spp. (Powdery Mildew), a Podosphaera leucotricha, a Podosphaera clandestine, a Phomopsis spp., a Phomopsis viticola, an Alternaria spp., an Alternaria tenuissima, an Alternaria porri, an Alternaria alternate, an Alternaria solani, an Alternaria tenuis, a Pseudomonas spp., a Pseudomonas syringae pv. Tomato, a Phytophthora spp., a Phytophthora infestans, a Phytophthora parasitica, a Phytophthora sojae, a Phytophthora capsici, a Phytophthora cinnamon, a Phytophthora fragariae, a Phytophthora spp., a Phytophthora ramorum, a Phytophthora palmivara, a Phytophthora nicotianae, a Phakopsora spp., a Phakopsora pachyrhizi, a Phakopsora meibomiae an Aspergillus spp., an Aspergillus flavus, an Aspergillus niger, a Uromyces spp., a Uromyces appendiculatus, a Cladosporium spp., a Cladosporium herbarum, a Rhizopus spp., a Rhizopus arrhizus, a Penicillium spp., a Rhizoctonia spp., a Rhizoctonia solani, a Rhizoctonia zeae, a Rhizoctonia oryzae, a Rhizoctonia caritae, a Rhizoctonia cerealis, a Rhizoctonia crocorum, a Rhizoctonia fragariae, a Rhizoctonia ramicola, a Rhizoctonia rubi, a Rhizoctonia leguminicola, a Macrophomina phaseolina, a Magnaorthe oryzae, a Mycosphaerella spp., Mycosphaerella graminocola, a Mycosphaerella fijiensis (Black sigatoga), a Mycosphaerella pomi, a Mycosphaerella citri, a Magnaporthe spp., a Magnaporthe grisea, a Monilinia spp., a Monilinia fruticola, a Monilinia vacciniicorymbosi, a Monilinia laxa, a Colletotrichum spp., a Colletotrichum gloeosporiodes, a Colletotrichum acutatum, a Colletotrichum Candidum, a Diaporthe spp., a Diaporthe citri, a Corynespora spp., a Corynespora Cassiicola, a Gymnosporangium spp., a Gymnosporangium juniperi-virginianae, a Schizothyrium spp., a Schizothyrium pomi, a Gloeodes spp., a Gloeodes pomigena, a Botryosphaeria spp., a Botryosphaeria dothidea, a Neofabraea spp., a Wilsonomyces spp., a Wilsonomyces carpophilus, a Sphaerotheca spp., a Sphaerotheca macularis, a Sphaerotheca pannosa, a Erysiphe spp., a Stagonospora spp., a Stagonospora nodorum, a Pythium spp., a Pythium ultimum, a Pythium aphanidermatum, a Pythium irregularum, a Pythium ulosum, a Pythium lutriarium, a Pythium sylvatium, a Venturia spp, a Venturia inaequalis, a Verticillium spp., a Ustilago spp., a Ustilago nuda, a Ustilago maydis, a Ustilago scitaminea, a Claviceps spp., a Claviceps puprrea, a Tilletia spp., a Tilletia tritici, a Tilletia laevis, a Tilletia horrid, a Tilletia controversa, a Phoma spp., a Phoma glycinicola, a Phoma exigua, a Phoma lingam, a Cocliobolus sativus, a Gaeumanomyces gaminis, a Colleototricum spp., a Rhychosporium spp., Rhychosporium secalis, a Biopolaris spp., a Helminthosporium spp., a Helminthosporium secalis, a Helminthosporium maydis, a Helminthosporium solai, and a Helminthosporium tritici-repentis, or combinations thereof.

### EXAMPLES

The following Examples have been included to provide guidance to one of ordinary skill in the art for practicing representative embodiments of the presently disclosed subject matter. In light of the present invention and the general level of skill in the art, those of skill can appreciate that the following Examples are intended to be exemplary only and that numerous changes, modifications, and alterations can be employed without departing from the scope of the presently disclosed subject matter.

### Example 1

### Identification of a Bacterial Isolate as a Bacillus Amyloliquefaciens through Sequence Analysis

A plant associated bacterial strain, designated herein as RTI301, was isolated from the rhizosphere soil of merlot vines growing at a vineyard in Long Island, N.Y. The 16S rRNA and the rpoB genes of the RTI301 strain were sequenced and subsequently compared to other known bacterial strains in the NCBI and RDP databases using BLAST. It was determined that the 16S RNA partial sequence of RTI301 (SEQ ID NO: 1) is identical to the 16S rRNA gene sequence of Bacillus amyloliquefaciens strain NS6 (KF177175), Bacillus amyloliquefaciens strain FZB42 (NR_075005), and Bacillus subtilis subsp. subtilis strain DSM 10 (NR_027552). It was also determined that the rpoB gene sequence of RTI301 (SEQ ID NO: 2) has sequence similarity to the same gene in Bacillus amyloliquefaciens subsp. plantarum TrigoCor1448 (CP007244) (99% sequence identity; 3 base pair difference); Bacillus amyloliquefaciens subsp. plantarum AS43.3 (CP003838) (99% sequence identity; 7 base pair difference); Bacillus amyloliquefaciens CC178 (CP006845) (99% sequence identity; 8 base pair difference), and Bacillus amyloliquefaciens FZB42 (CP000560) (99% sequence identity; 8 base pair difference). The RTI301 strain was identified as a Bacillus amyloliquefaciens. The differences in sequence for the rpoB gene at the DNA level indicate that RTI301 is a new strain of Bacillus amyloliquefaciens. The strain of Bacillus amyloliquefaciens RTI301 was deposited on 17 Apr. 2014 under the terms of the Budapest Treaty on the International Recognition of the Deposit of Microorganisms for the Purposes of Patent Procedure at the American Type Culture Collection (ATCC) in Manassas, Va., USA and bears the Patent Accession No. PTA-121165.

### Example 2

### Genes Related to Lantibiotic Biosynthesis in Bacillus Amyloliquefaciens RTI301 Strain

Further sequence analysis of the genome of the Bacillus amyloliquefaciens RTI301 strain revealed that this strain has genes related to lantibiotic biosynthesis, for which there are no homologues in the other closely related Bacillus amyloliquefaciens strains. This is illustrated in FIG. 1, which shows a schematic diagram of the genomic organization surrounding and including the lantibiotic biosynthesis operon found in Bacillus amyloliquefaciens RTI301. In FIG. 1, the top set of arrows represents protein coding regions for the RTI301 strain with relative direction of transcription indicated. For comparison, the corresponding regions for two Bacillus amyloliquefaciens reference strains, FZB42 and TrigoCor1448, are shown below the RTI301 strain. The genes in the lantibiotic synthesis operon in the RTI301 strain were initially identified using RAST and their identities then refined using BLASTp. The degree of amino acid identity of the proteins encoded by the genes of the RTI301 strain as compared to the two reference strains is indicated both by the degree of shading of the representative arrows as well as a percentage identity indicated within the arrow. It can be observed from FIG. 1 that there is a high degree of sequence identity in the genes from the 3 different strains in the regions surrounding the lantibiotic synthesis operon, but only a low degree of sequence identity within the lantibiotic synthesis operon (i.e., less than 40% within the lantibiotic synthesis operon but greater than 99% in the surrounding regions). BLASTn analysis of this cluster was performed against the non-redundant (nr)nucleotide database at NCBI and the analysis showed a high degree of homology in the 5′ and 3′ flanking regions to B. amyloliquefaciens strains (analogous to the high % similarity in FIG. 1). However, the lantipeptide biosynthetic cluster was unique to RTI301, and no significant homology to any previously sequenced DNA in the NCBI nr database was observed. Thus, this lantibiotic synthesis operon is a unique feature for Bacillus amyloliquefaciens strain RTI301.

### Example 3

### Growth Effects of Bacillus amyloliquefaciens RTI301 Isolate in Wheat

The effect of application of the bacterial isolate on early plant growth and vigor in wheat was determined. The experiment was performed by inoculating surface sterilized germinated wheat seeds for 2 days in a suspension of ˜2×10⁷ CFU/ml of the bacterium at room temperature under aeration in the dark (a control was also performed without bacteria). Subsequently, the inoculated and control seeds were planted in 6″ pots filled with sand. 10 seeds per pot and 1 pot per treatment were planted and watered as needed alternating with water and Modified Hoagland's solution. Pots were incubated in a lab windowsill at approximately 21° C. providing natural light/dark cycles for 13 days at which point plants were recovered and parameters measured. Dry weight was determined as a total weight per 9 plants resulting in a total average dry plant weight equal to 36.1 mg for the plants inoculated with the Bacillus amyloliquefaciens RTI301 strain versus a weight equal to 33.38 mg for the non-inoculated control which is an 8.1% increase in dry weight over the non-inoculated control. Photographs of the extracted plants after 13 days growth are shown in FIG. 2. FIG. 2A shows plants inoculated with RTI301 and FIG. 2B shows control plants.

In addition, another experiment was performed showing the beneficial effects of the Bacillus amyloliquefaciens RTI301 strain on early growth in wheat. The experiment was performed by inoculating surface sterilized germinated wheat seeds for 2 days in a suspension of 10⁺⁸ cfu/ml of the bacterium at room temperature under shaking. Subsequently, the inoculated seeds were planted in 1 gallon pots filled with PROMIX BX limed to pH 6.5. For each treatment 9 pots were seeded with 12 seeds planted at 2.5 cm depth. Pots were incubated in the greenhouse at 22° C. with light and dark cycle of 14/10 hrs and watered twice a week as needed. Photographs of the plants after 28 days growth are shown in FIG. 3. FIG. 3A shows plants inoculated with RTI301 and FIG. 3B shows control plants.

### Example 4

### Anti-Microbial Properties of Bacillus Amyloliquefaciens RTI301 Isolate

The antagonistic ability of the RTI301 isolate against major plant pathogens was measured in plate assays. A plate assay for evaluation of antagonism against plant fungal pathogens was performed by growing the bacterial isolate and pathogenic fungi side by side on 869 agar plates at a distance of 4 cm. Plates were incubated at room temperature and checked regularly for up to two weeks for growth behaviors such as growth inhibition, niche occupation, or no effect. In the case of screening for antagonistic properties against bacterial pathogens, the pathogen was first spread as a lawn on 869 agar plates. Subsequently, 20 μl aliquots of a culture of each of the isolates were spotted on the plate. Plates were incubated at room temperature and checked regularly for up to two weeks for an inhibition zone in the lawn around the positions where RTI301 had been applied. A summary of the antagonism activity is shown in Table I below. The RTI301 strain showed antagonisctic properties against a wide range of plant pathogenic microorganisms.

TABLE I
Antagonistic properties of B. amyloliquefaciens
strain RTI301 against major plant pathogens.

| Anti-Microbial Assays | RTI301 |
|---|---|
| Alternaria solani | ++ |
| Aspergillus flavus | ++ |
| Aspergillus nomius | +++ |
| Botrytis cinerea | +++ |
| Cercospora sojina | +++ |
| Fusarium colmorum | + |
| Fusarium graminearum | +++ |
| Fusarium oxysporum f. sp. Lycopersici | ++ |
| Fusarium oxysporum f. sp. cubense | ++ |
| Fusarium virguliforme | ++/+++ |
| Glomerella cingulata | +++ |
| Magnaporthe grisea | ++/+++ |
| Monilina fructicola | ++/+++ |
| Rhizoctonia solani | ++ |
| Sclerotinia homeocarpa | ++/+++ |
| Sclerotinia sclerotiorum | +++ |
| Septoria tritici | ++ |
| Stagonospora nodorum | ++/+++ |
| Phytophthora capsici | ++ |
| Pythium sylvatium | +−/+ |
| Pythium aphanidermatum | + |
| Erwinia amylovora | + |
| Erwinia carotovora | + |
| Pseudomonas syringae pv. tomato | − |
| Ralstonia solenacearum | ++ |
| Xanthomonas axonopodis | ++ |
| Xanthomonas euvesicatoria | ++ |

+++ very strong activity,
++ strong activity,
+ activity,
+− weak activity,
− no activity observed

### Example 5

### Phenotypic Traits of Bacillus Amyloliquefaciens RTI301 Isolate

In addition to the positive effects on plant growth and antagonistic properties, various phenotypic traits were also measured for the Bacillus amyloliquefaciens RTI301 strain and the data are shown below in Table II. The assays were performed according to the procedures described in the text below Table II.

TABLE II
Phenotypic Assays: phytohormone production, acetoin
and indole acetic acid (IAA), and nutrient Cycling of
Bacillus amyloliquefaciens RTI301 isolate.

| Characteristic Assays | RTI301 |
|---|---|
| Acid Production (Methyl Red) | − |
| Acetoin Production (MR-VP) | +++ |
| Chitinase activity | +− |
| Indole-3-Acetic Acid production | − |
| Protease activity | +++ |
| Phosphate Solubilization | +− |
| Phenotype | slimy cream, well-defined, |
| | round colonies |

+++ very strong,
++ strong,
+ some,
+− weak,
− none observed

Acid and Acetoin Test. 20 μl of a starter culture in rich 869 media was transferred to 1 ml Methy Red—Voges Proskauer media (Sigma Aldrich 39484). Cultures were incubated for 2 days at 30 C 200 rpm. 0.5 ml culture was transferred and 50 ul 0.2 g/l methyl red was added. Red color indicated acid production. The remaining 0.5 ml culture was mixed with 0.3 ml 5% alpha-napthol (Sigma Aldrich N1000) followed by 0.1 ml 40% KOH. Samples were interpreted after 30 minutes of incubation. Development of a red color indicated acetoin production. For both acid and acetoin tests non-inoculated media was used as a negative control (Sokol et al., 1979, Journal of Clinical Microbiology. 9: 538-540).

Indole-3-Acetic Acid. 20 μl of a starter culture in rich 869 media was transferred to 1 ml 1/10 869 Media supplemented with 0.5 g/l tryptophan (Sigma Aldrich T0254). Cultures were incubated for 4-5 days in the dark at 30 C, 200 RPM. Samples were centrifuged and 0.1 ml supernatant was mixed with 0.2 ml Salkowski's Reagent (35% perchloric acid, 10 mM FeCl3). After incubating for 30 minutes in the dark, samples resulting in pink color were recorded positive for IAA synthesis. Dilutions of IAA (Sigma Aldrich I5148) were used as a positive comparison; non inoculated media was used as negative control (Taghavi, et al., 2009, Applied and Environmental Microbiology 75: 748-757).

Phosphate Solubilizing Test. Bacteria were plated on Pikovskaya (PVK) agar medium consisting of 10 g glucose, 5 g calcium triphosphate, 0.2 g potassium chloride, 0.5 g ammonium sulfate, 0.2 g sodium chloride, 0.1 g magnesium sulfate heptahydrate, 0.5 g yeast extract, 2 mg manganese sulfate, 2 mg iron sulfate and 15 g agar per liter, pH7, autoclaved. Zones of clearing were indicative of phosphate solubilizing bacteria (Sharma et al., 2011, Journal of Microbiology and Biotechnology Research 1: 90-95).

Chitinase activity. 10% wet weight colloidal chitin was added to modified PVK agar medium (10 g glucose, 0.2 g potassium chloride, 0.5 g ammonium sulfate, 0.2 g sodium chloride, 0.1 g magnesium sulfate heptahydrate, 0.5 g yeast extract, 2 mg manganese sulfate, 2 mg iron sulfate and 15 g agar per liter, pH7, autoclaved). Bacteria were plated on these chitin plates; zones of clearing indicated chitinase activity (N. K. S. Murthy & Bleakley., 2012. “Simplified Method of Preparing Colloidal Chitin Used for Screening of Chitinase Producing Microorganisms”. The Internet Journal of Microbiology. 10(2)).

Protease Activity. Bacteria were plated on 869 agar medium supplemented with 10% milk. Clearing zones indicated the ability to break down proteins suggesting protease activity (Sokol et al., 1979, Journal of Clinical Microbiology. 9: 538-540).

### Example 6

### Bacillus Amyloliquefaciens RTI301 Antagonism of Uromyces Appendiculatus and Botrytis cinerea

Studies were performed in the greenhouse on soybean to determine the ability of the Bacillus amyloliquefaciens RTI301 strain to prevent and/or ameliorate the effects of the plant pathogen bean rust (Uromyces appendiculatus) and the plant pathogen Botrytis cinerea.

In a first set of experiments, different formulations of the B. amyloliquefaciens RTI301 strain were tested for foliar application to control the plant pathogens Uromyces appendiculatus and Botrytis cinerea. The experimental design and formulations were as follows:

Formulations:

B. amyloliquefaciens RTI301 spores in Spent Fermentation Broth (SFB), diluted by a factor of about 100 in water, and applied to foliage at a rate of 1×10⁸ cfu/ml.

B. amyloliquefaciens RTI301 spores in Spent Fermentation Broth (SFB), diluted by a factor of about 100 in water with added yeast extract, and applied to foliage at a rate of 1×10⁸ cfu/ml and about 0.2% yeast extract.

SERENADE OPTIMUM (BAYER CROP SCIENCE, INC) product was applied at a rate of B. subtilis strain QST713 spores at 1×10⁸ cfu/ml.

HORIZON (HORIZON AG-PRODUCTS) product was applied at a rate of 50 g a.i./ha (Tebuconazole).

BRAVO WEATHER STIK (SYNGENTA CROP PROTECTION, INC) product was applied at a rate of 500 g a.i./ha (Chlorothalonil).

TACTIC (LOVELAND PRODUCTS, INC) product was included in all the formulations listed above at the concentration of 0.1875% v/v.

Treatment Application Method:

A track sprayer was used to inoculate 21 day old common bean plants (having two trifoliates) with the various treatments listed above having a single overhead nozzle (TeeJet SS8001E Flat Fan) at a pressure=276 kPa (40 psi). The nozzle height was 36 cm (14″) above the bean plant leaves. The application volume was 200 L/ha and the number of repetitions in the experiment equaled six. The treatment plants were inoculated a single time along with control plants not receiving any treatment.

Infection Rate:

One day after treatment application, the test plants were infected with bean rust (Uromyces appendiculatus) at an inoculation rate of 200 k conidia/ml.

Nine days after infection with bean rust (Uromyces appendiculatus) the percent of disease control was evaluated for each of: RTI301 spores in Spent Fermentation Broth diluted 100 fold with water alone (“RTI301+1% SFB”), RTI301 spores in Spent Fermentation Broth diluted 100 fold with water plus yeast extract (“RTI301+1% SFB+Yeast Extract”), BRAVO WEATHER STIK, HORIZON, and SERENADE OPTIMUM according to the rates of application described above. The non-treated control (water only) resulted in 28% disease.

The results of the experiment are shown in Table III below. The results indicate that the addition of the yeast extract for the RTI301 strain resulted in about a 40% increase in disease control as compared to the RTI301 strain applied without the addition of yeast extract. The amount of disease control exhibited by RTI301+1% SFB+Yeast Extract was similar to that observed for SERENADE OPTIMUM when applied at the same rate (i.e., 1×10⁸ cfu/ml) even though the amount of SFB in the RTI301 formulation was relatively low at 1%, and the SFB can be expected to contain secreted compounds having antifungal activity.

Similar results were observed for an experiment performed in pepper plants using the same formulations and experimental design listed above to measure the amount of disease control exhibited by RTI301 for Pepper Botrytis Blight caused by the pathogen Botrytis cinerea (data not shown).

TABLE III
Results of B. amyloliquefaciens RTI301 control
of Bean Rust (Uromyces appendiculatus) as compared
to SERENADE OPTIMUM and chemical active agents when
formulated with and without yeast extract.

| | Percent |
|---|---|
| Treatment | Disease Control |
| RTI301 + 1% SFB (1 × 10⁸ cfu/ml) | 57 ab |
| RTI301 + 1% SFB + Yeast Extract (1 × 10⁸ | 96 cd |
| cfu/ml) | |
| BRAVO WEATHER STIK | 100 d |
| HORIZON | 100 d |
| SERENADE OPTIMUM (1 × 10⁸ cfu/ml) | 92 cd |
| Percent disease in non-treated control plants | |
| was 28% | |

The following experiment describes disease control of bean rust by RTI301 caused by the plant pathogen Uromyces appendiculatus. The experimental design and formulations were as follows:

Formulations:

Bacillus amyloliquefaciens RTI301 spores were in Spent Fermentation Broth (SFB), diluted by a factor of about 100 in water with added yeast extract, and applied to foliage at a rate of 1×10⁸ cfu/ml and about 0.2% yeast extract.

SERENADE OPTIMUM (BAYER CROP SCIENCE, INC) product was applied at a rate of spores at 1×10⁸ cfu/ml and 4×10⁸ cfu/ml.

TACTIC (LOVELAND PRODUCTS, INC) was applied at a concentration of 0.1875% v/v to all formulations.

HORIZON (HORIZON AG-PRODUCTS) was applied at a rate of 50 g a.i./ha (Tebuconazole).

Chlorothalonil was applied at a rate of 500 g a.i./ha.

Treatment Application Method:

A track sprayer was used to inoculate 21 day old bean plants (having two trifoliates) with the various treatments having a single overhead nozzle (TeeJet SS8001E Flat Fan) at a pressure=276 kPa (40 psi). The nozzle height was 36 cm (14″) above the soybean plant leaves. The application volume was 200 L/ha and the number of repetitions in the experiment equaled six. The treatment plants were inoculated a single time along with control plants not receiving any treatment.

Infection Rate:

One day after treatment application, the test plants were infected with bean rust (Uromyces appendiculatus) at an inoculation rate of 200 k conidia/ml. Ten days after infection with bean rust (Uromyces appendiculatus) the percent of disease control was evaluated for each of: the RTI301 spores in Spent Fermentation Broth (SFB) (applied at 1×10⁸ cfu/ml), SERENADE OPTIMUM (applied at 1×10⁸ cfu/ml), SERENADE OPTIMUM (applied at 4×10⁸ cfu/ml), Tebuconazole (applied at 50 g a.i./ha), and (Chlorothalonil applied at 500 g a.i./ha). TACTIC (applied at 0.1875%), also included as a control, was applied to all formulations. The check controls resulted in 23% disease. The results of the experiment are shown in Table IV below and in FIG. 4.

TABLE IV
Results of Bacillus amyloliquefaciens RTI301 control
of Bean Rust (Uromyces appendiculatus) as compared
to SERENADE OPTIMUM and other chemical active agents.

| | Percent |
|---|---|
| Treatment | Disease Control |
| RTI301 + SFB 1 × 10⁸ cfu/ml | 84 ab |
| SERENADE OPTIMUM 1 × 10⁸ cfu/ml | 95 a |
| 0.1875% TACTIC | 44 c |
| SERENADE OPTIMUM 4 × 10⁸ cfu/ml | 97 a |
| Tebuconazole | 100 a |
| Chlorothalonil | 100 a |
| Percent disease in checks - 23% | |

In another similar experiment, studies were performed in the greenhouse on bean plants to determine the ability of the Bacillus amyloliquefaciens RTI301 strain to prevent and/or ameliorate the effects of the plant pathogen Bean Rust (Uromyces appendiculatus) at varying rates of inoculation with the pathogen. The formulations and treatment methods used were the same as described for the previous experiment.

Infection Rate:

One day after treatment application, the test plants were infected with bean rust (Uromyces appendiculatus) at an inoculation rate ranging from 50 k to 300 k conidia/ml. Ten days after infection with bean rust (Uromyces appendiculatus) the percent of disease control was evaluated for each of: RTI301 spores in Spent Fermentation Broth (SFB) (applied at 1×10⁸ cfu/ml) and SERENADE OPTIMUM (applied at 1×10⁸ and 4×10⁸ cfu/ml), and HORIZON (Tebuconazole; applied at 50 g a.i./ha). TACTIC (applied at 0.1875%), also included as a control, was applied to all formulations. The percent disease in the check controls was 50 k=6%, 100 k=6%, 150 k=15%, 200 k=15%, and 300 k=7%. The results of the experiment are shown in Table V below and in FIG. 5.

TABLE V
Results of Bacillus amyloliquefaciens RTI301 ability to control bean rust
(Uromyces appendiculatus) after inoculation with the pathogen at varying concentration
as compared to SERENADE OPTIMUM and other chemical active agents.

| | | Percent Disease Control | | | | |
|---|---|---|---|---|---|---|
| Rate: | | RTI301 | SERENADE | | SERENADE | |
| Conidia/ | Percent | (1 × 10⁸) + | OPTIMUM | 0.1875% | OPTIMUM | |
| ml | Disease | SFB | (1 × 10⁸) | TACTIC | (4 × 10⁸) | Tebuconazole |
| 50K | 6 | 93 a | 94 a | 38 c | n/a | n/a |
| 100K | 6 | 76 ab | 91 a | 19 c | n/a | n/a |
| 150K | 15 | 95 a | 97 a | 51 bc | n/a | n/a |
| 200K | 15 | 92 a | 91 a | 22 c | 94 a | 100 a |
| 300K | 7 | 46 bc | n/a | 24 c | n/a | n/a |

### Example 7

### Bacillus Amyloliquefaciens RTI301 Antagonism of Cucurbit Disease Powdery Mildew in Cucumbers in Field Trials in Florida

Studies were performed in field trials of cucumber to determine the ability of the Bacillus amyloliquefaciens RTI301 strain to prevent and/or ameliorate the effects of the plant pathogen

Cucurbit Disease Powdery Mildew.

In total, 6 applications were made for each product with 5 to 7 day intervals between applications. The timing of the first application depended on the particular crop and ranged from at the time of planting, a fews weeks after crop emergence, at the time of flowering, upon disease emergence, or just prior to expectation of disease emergence.

Formulations:

SERENADE OPTIMUM (BAYER CROP SCIENCE, INC) was applied at a rate of 1400 g/ha, corresponding to 1.8×10⁺¹³ CFU/ha.

B. amyloliquefaciens RTI301 spores were in Spent Fermentation Broth (SFB) with added yeast extract and the application rate to foliage corresponded to the same colony forming units/ha (CFU/ha) as recommended for SERENADE OPTIMUM based on a 1400 g/ha application rate and at about 0.01% to 0.2% yeast extract.

TACTIC (LOVELAND PRODUCTS, INC) was applied at a concentration of 0.1875% v/v and included in all treatments.

LUNA EXPERIENCE (BAYER CROP SCIENCE, INC) was applied at a rate of 500 ga.i./ha (Fluopyram plus Tebuconazole fungicide).

BRAVO WEATHER STIK (SYNGENTA CROP PROTECTION, INC) was applied at a rate of 2240 ga.i./ha (Chlorothalonil).

The experimental design was as follows: Untreated control, RTI301+TACTIC, SERENADE OPTIMUM+TACTIC, and BRAVO WEATHER STIK+LUNA EXPERIENCE+TACTIC.

Treatment Application Method:

Six applications were made as described above. The application sprayer was set up to deliver 30 gallons per acre. The individual plots were sprayed at a ground speed of 4 mph using a CO₂ backpack sprayer with flat fan nozzles (8004 type) and each nozzle was spaced 18 inches apart.

Disease Scoring: The mean percent of disease severity was evaluated for 5 leaves of a plant for each of the treatments. Four plots from each treatment were evaluated for crop response and disease control of the powdery mildew. The trials were rated after each application just prior to the next application. The powdery mildew was from a natural infestation.

The results of the experiment are shown in Table VI below and indicate a similar control of powdery mildew in cucumbers as compared to SERENADE OPTIMUM when applied at the same rate as a stand alone biofungicide.

TABLE VI
Results of Bacillus amyloliquefaciens RTI301 control
of Powdery mildew in Cucumbers as compared to SERENADE
OPTIMUM and other chemical active agents.

| | | Mean % |
|---|---|---|
| | Powdery mildew in Cucumbers | disease Severity |
| 1 | Untreated control | 50 a |
| 2 | RTI301 + TACTIC | 25 b |
| 3 | SERENADE OPTIMUM + TACTIC | 30 b |
| 4 | BRAVO WEATHER STIK + LUNA | 10 c |
| | EXPERIENCE + TACTIC | |

### Example 8

### Bacillus Amyloliquefaciens RTI301 Antagonism of Bacterial Spot Tomato Disease (Xanthomonas) in Tomatoes in Field Trials

Studies were performed in field trials of tomato to determine the ability of the B. amyloliquefaciens RTI301 strain to prevent and/or ameliorate the effects of the plant pathogen Bacterial Spot Tomato Disease (Xanthomonas).

A total of 4 applications to the crop were made with 5 to 7 day intervals between applications. In the case of the program 4, which is combining the application of a biological with a chemical active ingredient, the first and third applications were made with the biological, while the second and fourth applications were made with the chemical.

Formulations:

SERENADE OPTIMUM was applied at a rate of 1400 g/ha, corresponding to 1.8×10⁺¹³ CFU/ha.

B. amyloliquefaciens RTI301 spores were in Spent Fermentation Broth (SFB) with added yeast extract and the application rate to foliage corresponded to the same colony forming units/ha as recommended for SERENADE OPTIMUM based on a 1400 g/ha application rate and at about 0.01% to 0.2% yeast extract.

TACTIC (LOVELAND PRODUCTS, INC) was applied at a concentration of 0.1875% v/v.

KOCIDE 3000 (DUPONT USA) was applied at a rate of 1850 g a.i./ha (Copper Hydroxide fungicide).

BRAVO WEATHER STIK (SYNGENTA CROP PROTECTION, INC) was applied at a rate of 2240 g a.i./ha (Chlorothalonil).

The experimental design was as follows: Untreated control, RTI301+TACTIC, SERENADE OPTIMUM+TACTIC, SERENADE OPTIMUM+KOCIDE 3000+TACTIC, and BRAVO WEATHER STIK+KOCIDE 3000+TACTIC.

Treatment Application Method:

Four separate treatment applications were delivered to the crop with a 5 to 7 interval between each application. The application sprayer was setup to deliver 40 gallons per acre. The individual plots were sprayed at a ground speed of 3 mph using a CO₂ backpack sprayer with cone nozzles and each nozzle was spaced 12 inches apart. The carrier to deliver the chemical was water mixed in a 2.5 liter bottle.

The disease severity was measured by evaluating the canopy. The mean percent of disease severity was evaluated in the middle of the plants for each of the treatments. The percentage disease control is based on considering the diseased, non-treated control plants as 100%. The data are shown below in Table VII. The treatments included: Untreated control, RTI301+TACTIC, SERENADE OPTIMUM+TACTIC, SERENADE OPTIMUM+KOCIDE 3000+TACTIC, and BRAVO WEATHER STIK+KOCIDE 3000+TACTIC.

The results of the experiment are shown in Table VII below. Bacterial Spot Tomato Disease (Xanthomonas) in tomatoes was controlled equally well by treatment with RTI301+TACTIC, SERENADE OPTIMUM+TACTIC, and BRAVO WEATHER STIK+KOCIDE 3000+TACTIC.

TABLE VII
Results of Bacillus amyloliquefaciens RTI301 control of Bacterial
Spot Tomato Disease (Xanthomonas) in tomatoes as compared
to SERENADE OPTIMUM and other chemical active agents.

| | | Mean % |
|---|---|---|
| | Severity of Bacterial Spot (mid canopy) | disease |
| 1 | Untreated control | 78 a |
| 2 | RTI301 + TACTIC | 42 c |
| 3 | SERENADE OPTIMUM + TACTIC | 39 c |
| 4 | SERENADE OPTIMUM + KOCIDE | 43 c |
| | 3000 + TACTIC | |
| 5 | BRAVO WEATHER STIK + KOCIDE | 37 c |
| | 3000 + TACTIC | |

### Example 9

### Bacillus amyloliquefaciens RTI301 Antagonism of Plant Pathogens in Field Trials

Studies were performed in field trials in Georgia of wheat, soybean, corn, cucumber, and tomato to determine the ability of the B. amyloliquefaciens RTI301 strain to prevent and/or ameliorate the effects of the plant pathogens wheat head scab, soybean rust, corn rust, cucumber powdery mildew, and bacterial spot in tomato caused by Xanthomonas sp.

Applications were made for each of RTI301 and SERENADE OPTIMUM using an application rate of 1400 g/ha for SERENADE OPTIMUM and an application rate for RTI301 corresponding to the same colony forming units/ha as recommended for SERENADE OPTIMUM based on a 1400 g/ha application rate.

One or more treatment applications were delivered to the crops with 5 to 7 day intervals between applications. The number of applications and timing of the first application depended on the particular crop and ranged from at the time of planting, a fews weeks after crop emergence, at the beginning of flowering, upon disease emergence, or just prior to expectation of disease emergence. The application sprayer was setup to deliver 20-30 gallons per acre (189 l/ha). The individual plots were sprayed at a ground speed of 3-4 mph using a CO₂ backpack sprayer with twin flat fan nozzles (8003 type or 8004 type).

For wheat head scab, a single treatment application was made to the plants at the beginning of crop flowering. Three days after treatment, the plants were artificially infected with the head scab pathogen Gibberella zeae (also known as Fusarium graminearum). Disease severity was measured by determining the percentage of the wheat head affected by the head scab (bleaching of the spikelets). The percentage control of disease severity is based on considering the percent disease severity in non-treated control plants as 100%. The data are shown below in Table VIII.

For soybean rust, 6 treatment applications were made to the plants. The initial application was delivered at the R1 stage of growth. This trial had a natural infestation. Disease severity was measured by evaluating stems and canopy. Specifically, six, 2 foot sections per plot were evaluated as subsamples and the severity was determined by estimating the percentage of the foliage that showed symptoms of rust from the entire canopy. The percentage control of disease severity is based on considering the percent disease severity in non-treated control plants as 100%. The data are shown below in Table VIII.

For corn rust, 6 treatment applications were made to the plants. This trial had a natural infestation. Disease severity was measured by evaluating the canopy. Six 2 foot sections per plot were evaluated as subsamples and the severity was determined by estimating the percentage of the foliage that showed symptoms of rust from the entire canopy. The disease severity was scored as number of hits per/1 m row. Four plots from each treatment were evaluated for crop response and disease control of corn rust. The percentage control of disease severity is based on considering the percent disease severity in non-treated control plants as 100%. The data are shown below in Table VIII.

For cucumber powdery mildew, 6 treatment applications were made to the plants about 3 weeks after crop emergence. The mean percent of disease severity was evaluated for 5 leaves of a plant for each of the treatments. Four plots from each treatment were evaluated for crop response and disease control of the powdery mildew. The trials were rated after each application just prior to the next application. The powdery mildew was from a natural infestation. The percentage control of disease severity is based on considering the percent disease severity in non-treated control plants as 100%. The data are shown below in Table VIII.

For tomato bacterial spot, 6 treatment applications were made to the plants. Disease severity was measured by looking at the canopy and estimating the percentage of the foliage affected. The percentage control of disease severity is based on considering the percent disease severity in non-treated control plants as 100%. The data are shown below in Table VIII.

TABLE VIII
Results of B. amyloliquefaciens RTI301 disease control of wheat
head scab, soybean rust, corn rust, cucumber powdery mildew, and
bacterial spot on tomato as compared to SERENADE OPTIMUM.

| | Control of | | |
|---|---|---|---|
| | Disease Severity* | | |
| | | SERENADE | Disease Severity |
| Pathogen | RTI301 | OPTIMUM | in Control Plants |
| Wheat Head Scab | 61% b | 42% d | 85% |
| Soybean Rust | 72% a | 53% b | 44% |
| Corn Rust | 69% b | 62% bc | 11% |
| Cucumber PMD⁺ | 50%b | 40%b | 50% |
| Bacterial Spot | 46%c | 50%c | 78% |
| on Tomato | | | |

*The percentage control of disease severity is based on considering the percent disease severity in non-treated control plants as 100%.
⁺PMD: Powdery mildew.

RTI301 controlled wheat head scab and soybean rust better than SERENADE OPTIMUM as measured by percent of the Untreated control. RTI301 was comparable to SERENADE OPTIMUM at controlling cucumber Powdery Mildew, corn rust, and Bacterial Spot on tomato as measured by percent of the Untreated control. No negative crop response was noted with RTI301 across the treatment application program.

### Example 10

### Bacillus Amyloliquefaciens RTI301 Antagonism of Sudden Death Syndrome Disease in Soybean

An experiment in soybean was performed to determine the ability of the B. amyloliquefaciens RTI301 strain to prevent and/or ameliorate the effects of sudden death syndrome disease in soybean. The experiment was performed as described below using spores of RTI301. For the experiment, the strain was sporulated in 2XSG in a 14 L fermenter. Spores were collected, washed and concentrated in H₂O at a concentration of 1.0×10¹⁰ CFU/mL.

An experiment in soybean was set up as follows: 1) seed was untreated; 2) seed was treated with a combination of CRUISERMAXX (insecticide plus fungicide, containing thiamethoxam, fludioxonil plus metalaxyl-M; SYNGENTA CROP PROTECTION, INC) and the thiophanate methyl fungicide, which is a typical soybean seed treatment (the combination of CRUISERMAXX and thiophanate methyl is referred to as “CHEM CONTROL”); and 3) seed was treated with CHEM CONTROL plus inoculated with 5.0×10⁺⁵ cfu/seed of strain RTI301.

A field trial was performed at Ames, Iowa on a soil that was inoculated with Fusarium virguliforme, the causal agent of soybean sudden death syndrome. F. virgulioforme was grown on moisten autoclaved grain seed. After the grain seed was covered with mycelia growth, the seed was air dried and subsequently ground up. The prepared ground inoculum was planted along with the soybean seed at the prescribed rate to ensure higher and more uniform infection rates. This disease infects early in the season although the symptoms do not manifest themselves until later in the season. After 119 days, the disease incidence, disease severity and the disease index were determined for soybean sudden death syndrome. In addition, the yield of soy beans was determined for each treatment.

The results in Table IX show that inoculation of the soybean seed with Bacillus amyloliquefaciens RTI301 had a positive effect on disease control, as measured by various parameters, and on the overall yield of soybean when compared to seeds that were treated with the CHEM CONTROL alone, and resulted in a 7.8% increase in soybean yield (from 55.2 to 59.5 bushels per acre) over the CHEM CONTROL alone.

TABLE IX
Results of B. amyloliquefaciens RTI301 disease
control of Sudden Death Syndrome in soybean compared
to seeds treated with CRUISERMAXX plus the thiophanate
methyl fungicide (referred to as CHEM Control), which
is a typical soybean seed treatment.

| | | SDS | % SDS | Index | |
|---|---|---|---|---|---|
| | | Incidence | Severity | SDS | Yield |
| | | 119 DP-1 | 119 DP-1 | 119 DP-1 | bu/acre |
| 1 | UNTREATED SEED | 42 | 91 | 3.8 | 53.9 |
| 2 | CHEM. CONTROL | 31 | 84 | 2.8 | 55.2 |
| 3 | CHEM. CONTROL + | 19 | 59 | 1.2 | 59.5 |
| | RTI301 | | | | |

The disease incidence, disease severity, disease index and yield were determined for soybean 119 days after planting in conditions where the soil was inoculated with Fusarium virguliforme, the causal agent of soybean sudden death syndrome.

### Example 11

### B. amyloliquefaciens RTI301 Antagonism of Brownish Grey Mildew (Botrytis cinerea) in Tomatoes in Field Trials in Italy

Studies were performed in field trials of tomato to determine the ability of the B. amyloliquefaciens RTI301 strain to prevent and/or ameliorate the effects of the plant pathogen Brownish Grey Mildew (Botrytis cinerea).

A total of 4 applications to the crop were made with 7 day intervals between applications.

Formulations:

SERENADE MAX was applied at a rate of 4000 g/ha, corresponding to 2.0×10⁺¹⁴ CFU/ha of Bacillus subtilis strain QST713.

B. amyloliquefaciens RTI301 spores were in spent fermentation broth (SFB) with added yeast extract and the application rate was 2.0×10⁺¹³ CFU/ha at about 0.01% to 0.2% yeast extract.

The first two applications to the crop were made with SWITCH (cyprodinil 375 g/kg plus fludioxonil 250 g/kg; SYNGENTA CROP PROTECTION, INC) at a rate of 0.8 kg/ha, followed by two applications of SIGNUM (boscalid 267 g/kg plus pyraclostrobin 67 g/kg; BAYER CROP SCIENCE, INC) at a rate of 1.8 kg/ha. This is referred to herein as the “FARMER's program”.

SILWET L77 (HELENA CHEMICAL), a nonionic organosilicone surfactant, was used in all treatments, except for SERENADE MAX, at a rate of 0.15 liter per 100 liter spray solution.

The experimental design was as follows: Untreated control (UTC), FARMER's program+SILWET L77, RTI301+SILWET L77, and SERENADE MAX.

Treatment Application Method:

Two independent field trials were performed, each with 4 replicates, and the results of both trials were combined and presented as average results. Four separate treatment applications were delivered to the crop with a 7 day interval between each application. Three days before the start of the treatments, all plots, including the untreated control, were treated with SWITCH to suppress initial disease development. The application sprayer was setup to deliver 53 gallons per acre. The individual plots were sprayed at a ground speed of 1.7 mph (0.8 m/s or 2.9 km/h) using a CO₂ backpack sprayer with cone nozzles and each nozzle was spaced 2 inches apart (5.5 cm).

All tomatoes were harvested, counted, weighted and separated into marketable or diseased to determine yield. The disease incidence (% of fruit affected by Brownish Grey Mildew) was measured by evaluating the fruits for each of the treatments at harvest on 5 separate dates and expressed as “Area Under Disease Pressure Curve” (AUDPC). The disease incidence and cumulative yield data are shown below in Table X. The disease incidence in the UTC as a function of time is shown in the graphs below (FIG. 6), and shows that the disease pressure increased during the course of both trials, ending with very high disease pressure, i.e., 51.5% and 44.5% of fruits infested for each of the two trials, respectively.

The results show that the best control of Brownish Grey Mildew (Botrytis cinerea) on tomatoes was observed for B. amyloliquefaciens RTI301 and the FARMER's program based on chemical active agents, and outperformed the treatment using SERENADE MAX having a 10-folder higher concentration of Bacillus subtilis strain QST713 than the RTI301.

TABLE X
Results of B. amyloliquefaciens RTI301 control of Brownish Grey
Mildew (Botrytis cinerea) on tomatoes as compared to SERENADE
MAX and the FARMER'S program based on chemical active agents.
The results are the average of two independent field trials.

| | | Average % | Cumulative | Statistical |
|---|---|---|---|---|
| | Severity of Brownish | disease as | yield | relevance |
| | Grey Mildew | AUDPC | (kg) | (P of 0.1) |
| 1 | Untreated control | 34.6 | 9.5 | c |
| 2 | FARMER'S program + | 3.7 | 20 | a |
| | SILWET L77 | | | |
| 3 | RTI301 + SILWET L77 | 3.3 | 18.6 | a |
| 4 | SERENADE MAX | 13.1 | 16.1 | b |

### Example 12

### B. amyloliquefaciens RTI301 Antagonism of Brownish Grey Mildew (Botrytis cinerea) in Strawberry in Field Trials in Italy and Spain

Studies were performed in field trials of strawberry to determine the ability of the B. amyloliquefaciens RTI301 strain to prevent and/or ameliorate the effects of the plant pathogen Brownish Grey Mildew (Botrytis cinerea).

A total of 4 applications to the crop were made with 7 day intervals between applications.

Formulations:

SERENADE MAX was applied at a rate of 4000 g/ha, corresponding to 2.0×10⁺¹⁴ CFU/ha of Bacillus subtilis strain QST713.

B. amyloliquefaciens RTI301 spores were in Spent Fermentation Broth (SFB) with added yeast extract and the application rate was 2.0×10⁺¹³ CFU/ha at about 0.01% to 0.2% yeast extract. In addition SILWET L77 (HELENA CHEMICAL), a nonionic organosilicone surfactant, was added at a rate of 0.15 liter per 100 liter spray solution.

The first two applications to the crop were made with SWITCH (cyprodinil 375 g/kg plus fludioxonil 250 g/kg; SYNGENTA CROP PROTECTION, INC) at a rate of 0.8 kg/ha, followed by two applications of SIGNUM (boscalid 267 g/kg plus pyraclostrobin 67 g/kg; BAYER CROP SCIENCE, INC) at a rate of 1.8 kg/ha. This is referred to herein as the “FARMER's program”.

The experimental design was as follows: Untreated control (UTC), FARMER's program, RTI301+SILWET L77, and SERENADE MAX.

Treatment Application Method:

Four independent field trials were performed, each with 4 replicates, and the results of the trials were combined and presented as average results. Four separate treatment applications were delivered to the crop with a 7 day interval between each application. Three days before the start of the treatments, all plots, including the untreated control, were treated with SWITCH to suppress initial disease development. The application sprayer was setup to deliver 53 to 107 gallons per acre depending on crop density at application. The individual plots were sprayed at a ground speed of 0.56 mph (0.25 m/s or 0.9 km/h) using a CO₂ backpack sprayer with cone nozzles and each nozzle was spaced 2 inches apart (5.5 cm).

All strawberries were harvested, counted, weighted and separated into marketable or diseased to determine yield. The disease incidence (% of fruit affected by Brownish Grey Mildew) was measured by evaluating the fruits for each of the treatments at harvest on 6 separate dates and expressed as “Area Under Disease Pressure Curve” (AUDPC). The disease incidence and % increase in yield versus the untreated control are shown below in Table XI. The disease incidence in the UTC as a function of time is shown in the graphs below (FIG. 7), and shows that the disease pressure progressed during the trials reaching highest disease pressure of 20% to 45% of fruits infested.

The results in Table XI below show that improved control of Brownish Grey Mildew (Botrytis cinerea) on strawberry over the untreated control was observed for all three treatments, B. amyloliquefaciens RTI301, SERENADE MAX, and the FARMER's program, with a slightly higher numerical increase of yield for the treatment with RTI301.

TABLE XI
Results of B. amyloliquefaciens RTI301 control of Brownish Grey
Mildew (Botrytis cinerea) on strawberry as compared to SERENADE
MAX and the FARMER'S program based on chemical active agents.
The results are the average of four independent field trials.

| | | Average % | % Yield | Statistical |
|---|---|---|---|---|
| | Severity of Brownish | disease as | increase | relevance |
| | Grey Mildew | AUDPC | over UTC | (P of 0.1) |
| 1 | Untreated control (UTC) | 19.1 | 0 | a |
| 2 | FARMER'S program | 13.2 | 11.5 | b |
| 3 | RTI301 + SILWET L77 | 10.8 | 15.7 | b |
| 4 | SERENADE MAX | 12.7 | 14.9 | b |

### Example 13

### Corn Seed Treatment with B. amyloliquefaciens RTI301

Experiments were performed to investigate the effect on plant growth and development in corn after treatment of the plant seed with B. amyloliquefaciens RTI301 strain.

Specifically, an experiment in corn was set up as follows: 1) seed was untreated; 2) seed was treated with a combination of MAXIM (broad-spectrum seed treatment fungicide fludioxonil as its active ingredient at 0.0625 mg/seed; SYNGENTA CROP PROTECTION, INC), APRON XL (active ingredient metalaxyl-M at 0.0625 mg/seed); SYNGENTA CROP PROTECTION, INC) and PONCHO (Clothianidin insecticide at 0.25 mg/seed; BAYER CROPSCIENCE, INC), which is a typical corn seed treatment (the combination of MAXIM, APRON XL and PONCHO is referred to as “CHEM CONTROL”); and 3) seed was treated with CHEM CONTROL plus inoculated with 5.0×10⁺⁵ cfu/seed of strain RTI301. Three trials were performed with 5 replicates per treatment per trial at field sites located in Shawneetown, Ill. The conditions for the 3 trials were natural disease pressure or inoculation of the soil with one of Fusarium graminearum or Rhizoctonia. Fusarium graminearum and Rhizoctonia were grown separately on moistened autoclaved grain seed and then air dried. The dried inoculum used in a selected trial was mixed with the seed at a prescribed rate to provide infection when the seed commenced to grow.

The average corn yield results (bushels per acre) for the field trials are presented in Table XII below. The results in Table XII show that inoculation with the CHEM CONTROL plus B. amyloliquefaciens RTI301 had an effect on the overall average yield of corn under all 3 conditions when compared to seeds that were treated with the CHEM CONTROL alone. The statistical relevance (as letters) is based on P=0.1. Notably, a very large yield benefit of 40.1 bushels per acre was observed with RTI301 plus chemical control over the chemical control alone for the trials inoculated with Rhizoctonia. In addition, a yield increase of 3.3 bushels per acre and 8.4 bushels per acre were recorded for trials artificially inoculated with Fusarium graminearum and natural disease pressure, respectively. In summary, treatment with the chemical control plus RTI301 resulted in an increase in yield for all 3 trials and resulted in a very large increase in yield for the trials in which the corn plants were inoculated with Rhizoctonia.

TABLE XII
Yield and yield increase in bushels per acre of untreated corn (UTC), corn treated
with the chemical control (CC), and corn treated with the chemical control
plus Bacillus amyloliquefaciens RTI301 at a rate of 5 × 10⁺⁵ cfu/seed.

| | | Natural | | | Rhizoctonia | | | Fusarium graminearum | | |
|---|---|---|---|---|---|---|---|---|---|---|
| | | Bu/Acre | | Increase | Bu/Acre | | Increase | Bu/Acre | | Increase |
| 1 | UTC | 170.7 | e | −23.8 | 142 | e | −22.0 | 187.0 | a | −11.3 |
| 2 | CC | 194.5 | bcd | 0.0 | 164 | de | 0.0 | 198.3 | a | 0.0 |
| 3 | CC + RTI 301 | 202.9 | a-d | 8.4 | 204.1 | bc | 40.1 | 201.6 | a | 3.3 |
| | (5 × 10⁺⁵ cfu/seed) | | | | | | | | | |

### Example 14

### B. amyloliquefaciens RTI301 Antagonism of Fungal Pathogens in Combination with FRACTURE

Studies were performed with an in vitro plate assay to determine the antagonistic ability of the B. amyloliquefaciens RTI301 strain to enhance the performance of FRACTURE (CONSUMO EM VERDE (CEV), BIOTECNOLOGIA DAS PLANTAS S.A., PORTUGAL) to control fungal phytopathogens. FRACTURE is a plant extract-based formulation containing 20% BLAD polypeptide as active ingredient. BLAD polypeptide is a fragment of a naturally occurring seed storage protein in sweet lupine (Lupinus albus) that acts on susceptible fungal pathogens by causing damage to the fungal cell wall and disrupting the inner cell membrane.

A plate assay for evaluation of antagonism against plant fungal pathogens was performed by growing the bacterial isolate and pathogenic fungi side by side on 869 agar plates or on 869+1% FRACTURE agar plates. On opposite sides of each plate, 20 μl of a RTI301 spore solution containing 1×10⁸ CFU/ml or 1×10⁹ CFU/ml were spotted at a distance of 4 cm from the center of the plate. Subsequently, 20 μl of a fungal spore solution or an agar plug inoculated with fungal mycelium was placed in the center of the plate. Plates were incubated at 25° C. for 7 days and checked regularly for growth behaviors such as growth inhibition, niche occupation, or no effect.

The results of the antagonism activities provided by B. amyloliquefaciens RTI301 against Fusarium graminearum and Fusarium oxysporum fc. cubense in combination with FRACTURE are illustrated in FIGS. 8A-8F (Fusarium graminearum) and FIGS. 9A-9F (Fusarium oxysporum fc. cubense).

FIGS. 8A-8F are images of the plate assay showing control of Fusarium graminearum by B. amyloliquefaciens RTI301 in the presence and absence of FRACTURE. A) growth of Fusarium graminearum on a 869 agar plate; B) growth of Fusarium graminearum on a 869 agar plate in the presence of 20 μl of a RTI301 spore solution containing 1×10⁸ CFU/ml (left) or 1×10⁹ CFU/ml (right), respectively; C) growth of 20 μl of a B. amyloliquefaciens RTI301 spore solution containing 1×10⁸ CFU/ml (left) or 1×10⁹ CFU/ml (right), respectively, on a 869 agar plate; D) growth of Fusarium graminearum on a 869+1% FRACTURE agar plate; E) growth of Fusarium graminearum on a 869+1% FRACTURE agar plate in the presence of 20 μl of a RTI301 spore solution containing 1×10⁸ CFU/ml (left) or 1×10⁹ CFU/ml (right), respectively; F) growth of 20 μl of a B. amyloliquefaciens RTI301 spore solution containing 1×10⁸ CFU/ml (left) or 1×10⁹ CFU/ml (right), respectively, on a 869+1% FRACTURE agar plate.

FIGS. 9A-9F are images of the plate assay showing control of Fusarium oxysporum fc. cubense by B. amyloliquefaciens RTI301 in the presence and absence of FRACTURE. A) growth of Fusarium oxysporum fc. cubense on a 869 agar plate; B) growth of Fusarium oxysporum fc. cubense on a 869 agar plate in the presence of 20 μl of a RTI301 spore solution containing 1×10⁸ CFU/ml (left) or 1×10⁹ CFU/ml (right), respectively; C) growth of 20 μl of a B. amyloliquefaciens RTI301 spore solution containing 1×10⁸ CFU/ml (left) or 1×10⁹ CFU/ml (right), respectively, on a 869 agar plate; D) growth of Fusarium oxysporum fc. cubense on a 869+1% FRACTURE agar plate; E) growth of Fusarium oxysporum fc. cubense on a 869+1% FRACTURE agar plate in the presence of 20 μl of a RTI301 spore solution containing 1×10⁸ CFU/ml (left) or 1×10⁹ CFU/ml (right), respectively; F) growth of 20 μl of a B. amyloliquefaciens RTI301 spore solution containing 1×10⁸ CFU/ml (left) or 1×10⁹ CFU/ml (right), respectively, on a 869+1% FRACTURE agar plate.

The results show that the presence of 1% FRACTURE in the 869 agar medium did not inhibit the growth of B. amyloliquefaciens RTI301. The lack of inhibition of RI301 was in contrast to that observed for other B. amyloliquefaciens strains where the presence of the 1% FRACTURE did inhibit growth of the strain. Furthermore, although the addition of 1% FRACTURE to the 869 medium resulted in reduced growth of Fusarium graminearum and Fusarium oxysporum fc. cubense, full inhibition of fungal growth was not achieved. However, the presence of B. amyloliquefaciens RTI301 resulted in additional inhibition of fungal growth for both Fusarium graminearum and Fusarium oxysporum fc. cubense. Therefore, B. amyloliquefaciens RTI301 can be used to enhance the performance of FRACTURE. A similar result was also observed for the control of Aspergillus flavus (data not shown).

### Example 15

### Identification of New Metabolites produced by Bacillus Amyloliquefaciens RTI301 Isolate

It has been previously reported that five classes of Fengycin-type metabolites and Dehydroxyfengycin-type metabolites are produced by microbial species including Bacillus amyloliquefaciens (see, for example, Li, Xing-Yu, et al. , 2013, J. Microbiol. Biotechnol. 23(3), 313-321; Pecci Y, et al. 2010 Mass Spectrom., 45(7):772-77). These metabolites, cyclic lipopeptides, are cyclic peptide molecules that also contain a fatty acid group. The five classes of Fengycin- and Dehydroxyfengycin-type metabolites are referred to as A, B, C, D and S. The backbone structure of these metabolites as well as the specific amino acid sequence for each of the five classes is shown in FIG. 10.

The Fengycin- and Dehydroxyfengycin-type metabolites produced by Bacillus amyloliquefaciens RTI301 were analyzed using UHPLC-TOF MS. The molecular weights of the Fengycin-type metabolites produced by the RTI301 strain after 6 days growth in M2 medium at 30° C. were compared to the theoretical molecular weights expected for the Fengycin- and Dehydroxyfengycin-type metabolites. In addition, to determine the amino acid composition of the various Fengycin-type metabolites produced by the RTI301 strain, peptide sequencing using LC-MS-MS was performed on each of the Fengycin-type metabolites previously identified via UHPLC-TOF MS. In this manner, it was determined that Bacillus amyloliquefaciens RTI301 produces Fengycin A, B and C and Dehydroxyfengycin A, B and C. Surprisingly, in addition to these known compounds, it was determined that the RTI301 strain also produces previously unidentified derivatives of these compounds.

For example, it was determined that the Bacillus amyloliquefaciens RTI301 strain produces Fengycin-like and Dehydroxyfengycin-like compounds where the L-isoleucine at position 8 of the cyclic peptide chain (referred to as X₃ in FIG. 10) is replaced by L-methionine. The new classes of Fengycin and Dehydroxyfengycin are referred to herein as MA, MB and MC, referring to derivatives of classes A, B and C in which the L-isoleucine at X₃ in FIG. 10 has been replaced by L-methionine. The newly identified molecules are shown in bold in FIG. 10 and in Table XIII below.

It was further determined that the RTI301 strain produces an additional class of Fengycin and Dehydroxyfengycin that has not been previously identified. In this class, the L-isoleucine of Fengycin B and Dehydroxyfengycin B (position X₃ in FIG. 10) is replaced by L-homo-cysteine (Hcy). These previously unidentified Fengycin and Dehydroxyfengycin metabolites are referred to herein as Fengycin H and Dehydroxyfengycin H and are shown in in FIG. 6 and Table XIII.

It was further determined that the RTI301 strain produces an additional class of previously unidentified Fengycin and Dehydroxyfengycin metabolites. In this class, the amino acid at position 4 of the cyclic peptide backbone structure (position X₁ in FIG. 10) is replaced by L-isoleucine. These previously unidentified metabolites are referred to herein as Fengycin I and Dehydroxyfengycin I and are shown in FIG. 10 and in Table XIII.

A summary of the amino acid sequences for the previously reported Fengycin- and Dehydroxyfengycin-type lipopeptides and the newly identified metabolites is provided in Table XIII below.

TABLE XIII
Summary of UHPLC-TOF MS identification of Fengycin-type lipopeptides in Bacillus amyloliquefaciens RTI301.

| | | | | | Ring | Theoretical C16 | Theoretical C16 | Observed |
|---|---|---|---|---|---|---|---|---|
| Homolog | X₁ | X₂ | X₃ | R | Mass | Molecular Formula | [M + H]⁺ | RTI301 |
| Fengycin A | Ala | Thr | Ile | OH | 1080.6 | C₇₂H₁₁₀N₁₂O₂₀ | 1463.8 | C15, C16, C17 |
| Fengycin B | Val | Thr | Ile | OH | 1108.7 | C₇₄H₁₁₄N₁₂O₂₀ | 1491.8 | C14, C15, C16, C17 |
| Fengycin C | Aba | Thr | Ile | OH | 1094.6 | C₇₃H₁₁₂N₁₂O₂₀ | 1477.8 | C14, C15, C16, C17 |
| Fengycin D | Val | Thr | Val | OH | 1094.6 | C₇₃H₁₁₂N₁₂O₂₀ | 1477.8 | C14, C15, C16, C17 |
| Fengycin S | Val | Ser | Ile | OH | 1094.6 | C₇₃H₁₁₂N₁₂O₂₀ | 1477.8 | C14, C15, C16, C17 |
| Fengycin MA | Ala | Thr | Met | OH | 1098.7 | C₇₁H₁₀₈N₁₂O₂₀S | 1481.8 | C15, C16, C17 |
| Fengycin MB | Val | Thr | Met | OH | 1126.8 | C₇₃H₁₁₂N₁₂O₂₀S | 1509.8 | C14, C15, C16 |
| Fengycin MC | Aba | Thr | Met | OH | 1112.7 | C₇₂H₁₁₀N₁₂O₂₀S | 1495.8 | C14, C15, C16, C17 |
| Fengycin H | Val | Thr | Hcy | OH | 1112.7 | C₇₂H₁₁₀N₁₂O₂₀S | 1495.8 | C14, C15, C16, C17 |
| Fengycin I | Ile | Thr | Ile | OH | 1122.8 | C₇₅H₁₁₆N₁₂O₂₀ | 1505.8 | C16, C17 |
| Dehydroxyfengycin A | Ala | Thr | Ile | H | 1080.6 | C₇₂H₁₁₀N₁₂O₁₉ | 1447.8 | C15, C16, C17 |
| Dehydroxyfengycin B | Val | Thr | Ile | H | 1108.7 | C₇₄H₁₁₄N₁₂O₁₉ | 1475.8 | C14, C15, C16, C17 |
| Dehydroxyfengycin C | Aba | Thr | Ile | H | 1094.6 | C₇₃H₁₁₂N₁₂O₁₉ | 1461.8 | C14, C15, C16, C17 |
| Dehydroxyfengycin D | Val | Thr | Val | H | 1094.6 | C₇₃H₁₁₂N₁₂O₁₉ | 1461.8 | C14, C15, C16, C17 |
| Dehydroxyfengycin S | Val | Ser | Ile | H | 1094.6 | C₇₃H₁₁₂N₁₂O₁₉ | 1461.8 | C14, C15, C16, C17 |
| Dehydroxyfengycin MA | Ala | Thr | Met | H | 1098.7 | C₇₁H₁₀₈N₁₂O₁₉S | 1465.7 | C14 |
| Dehydroxyfengycin MB | Val | Thr | Met | H | 1126.8 | C₇₃H₁₁₂N₁₂O₁₉S | 1493.8 | C15 |
| Dehydroxyfengycin MC | Aba | Thr | Met | H | 1112.7 | C₇₂H₁₁₀N₁₂O₁₉S | 1479.8 | C15 |
| Dehydroxyfengycin H | Val | Thr | Hcy | H | 1112.7 | C₇₂H₁₁₀N₁₂O₁₉S | 1479.8 | C14, C15, C16 |
| Dehydroxyfengycin I | Ile | Thr | Ile | H | 1122.8 | C₇₅H₁₁₆N₁₂O₁₉ | 1489.9 | C15, C16, C17 |

### Example 16

### Antimicrobial Activity of Isolated Lipopeptide Metabolites of RTI301

Antagonistic lipopeptides from B. amyloliquefaciens strain RTI301 were isolated from RTI301 spent fermentation broth and shown to retain their activity.

In this experiment, the Bacillus amyloliquefaciens RTI301 culture supernatant was acidified to pH 2 according to the procedure described in Smyth, T J P et al., 2010, “Isolation and Analysis of Lipopeptides and High Molecular Weight Biosurfactants.” In: Handbook of Hydrocarbon and Lipid Microbiology, K. N. Timmis (Editor). pp 3687-3704. The recovery of the lipopeptides was analyzed by UHPLC-TOF MS, and their antagonistic activity against Botrytis cinerea and Fusarium graminearum were tested.

The RTI301 was cultured in M2 sporulation medium for six days at 30° C., and the spent fermentation broth (301-SFB) was centrifuged at 18,514 g for 20 min to remove the spores. The supernatant was subsequently acidified to pH 2.0 by addition of concentrated HCl, and overnight precipitated at 4° C. The sample was subsequently centrifuged at 18,514 g for 20 min to obtain the solid crude lipopeptides. The pellet was lyophilized overnight, dissolved in the original volume of M2 medium, and analyzed by LCMS. The masses of iturins (C14, C15, C16), surfactins (C12, C13, C14, C15, C16, C17), fengycins (A, B, C, D, S) were extracted, integrated, and summed up to compare relative abundance of the lipopeptides from each sample. FIG. 11 is a graph showing the percentage of recovered lipopeptides from the RTI301 spent fermentation broth (SFB) after the acid precipitation. The terms “301-AP-Pellet” and “301-AP-Supernatant” refer to the resuspended pellet and supernatant, respectively, obtained after acid precipitation of the centrifuged SFB. The results in the graph in FIG. 11 show that 80% of the total amount of lipopeptides was recovered by acid precipitation. For iturin 35% was precipitated, while 59% of the iturin was not recovered via the acid precipitation method. Surfactin and fengycin were 100% recovered using acid precipitation.

To confirm that the LCMS results correlated with antagonistic activity, a bioassay was performed with the same samples analyzed by LCMS. For the bioassay, 20 μl of Botrytis cinerea or Fusarium graminearum inoculum was spotted in the middle of plate with 301-AP-Pellet sample spotted in 10 μl, 20 μl, and 40 μl aliquots. The antifungal activity was checked after 5 days or 7 days incubation at 30° C. for Botrytis cinerea and Fusarium graminearum plates, respectively. The results showed that the acid precipitated sample (301-AP-Pellet) has a similar level of antagonistic activity as the starting spent fermentation broth against both Botrytis cinerea and Fusarium graminearum. The bioassay results are well correlated with the LCMS data.

### REFERENCES

All publications, patent applications, patents, and other references cited herein are incorporated herein by reference in their entireties.

Although the foregoing subject matter has been described in some detail by way of illustration and example for purposes of clarity of understanding, it will be understood by those skilled in the art that certain changes and modifications can be practiced within the scope of the claims.

That which is claimed:

1. A composition comprising a biologically pure culture of Bacillus amyloliquefaciens RTI301 deposited as ATCC No. PTA-121165, or a mutant thereof having all the identifying characteristics thereof, for application to a plant for one or both of benefiting plant growth or conferring protection against a pathogenic infection in a susceptible plant.

2. The composition of claim 1, wherein the composition is in the form of a liquid, a dust, a dry wettable powder, a spreadable granule, or a dry wettable granule.

3. The composition of claim 1, wherein the composition is in the form of a liquid and the Bacillus amyloliquefaciens RTI301 is present at a concentration of from about 1.0×10⁸ CFU/ml to about 1.0×10¹² CFU/ml.

4. The composition of claim 1, wherein the composition is in the form of a dust, a dry wettable powder, a spreadable granule, or a dry wettable granule and the Bacillus amyloliquefaciens RTI301 is present in an amount of from about 1.0×10⁸ CFU/g to about 1.0×10¹² CFU/g.

5. The composition of claim 1, wherein the composition is in the form of an oil dispersion and the Bacillus amyloliquefaciens RTI301 is present at a concentration of from about 1.0×10⁸ CFU/ml to about 1.0×10¹² CFU/ml.

6. The composition of claim 1, wherein the Bacillus amyloliquefaciens RTI301 is in the form of spores or vegetative cells.

7. The composition of claim 1, further comprising one or a combination of a carrier, a surfactant, a dispersant, or a yeast extract.

8. A plant seed coated with a composition comprising:
spores of a biologically pure culture of Bacillus amyloliquefaciens RTI301 deposited as ATCC No. PTA-121165, or a mutant thereof having all the identifying characteristics thereof, present in an amount suitable to benefit plant growth and/or to confer protection against a pathogenic infection in a susceptible plant.

9. The plant seed of claim 8, wherein the composition comprises an amount of Bacillus amyloliquefaciens spores from about 1.0×10² CFU/seed to about 1.0×10⁹ CFU/seed.

10. The plant seed of claim 8, wherein the composition further comprises one or a combination of a microbial, a biological, or a chemical insecticide, fungicide, nematicide, bacteriocide, or plant growth regulator present in an amount suitable to benefit plant growth and/or to confer protection against a pathogenic infection in a susceptible plant.

11. The plant seed of claim 10, wherein the fungicide is one or more of benzovindiflupyr, anitiperonosporic, ametoctradin, amisulbrom, copper salts including copper hydroxide, copper oxychloride, copper sulfate, and copper persulfate, boscalid, thiflumazide, flutianil, furalaxyl, thiabendazole, benodanil, mepronil, isofetamid, fenfuram, bixafen, fluxapyroxad, penflufen, sedaxane, coumoxystrobin, enoxastrobin, flufenoxystrobin, pyraoxystrobin, pyrametostrobin, triclopyricarb, fenaminstrobin, metominostrobin, pyribencarb, meptyldinocap, fentin acetate, fentin chloride, fentin hydroxide, oxytetracycline, chlozolinate, chloroneb, tecnazene, etridiazole, iodocarb, prothiocarb, Bacillus subtilis syn., Bacillus amyloliquefaciens including strains QST 713, FZB24, MBI600, and D747, extract from Melaleuca alternifolia, extract from Lupinus albus doce, BLAD polypeptide, pyrisoxazole, oxpoconazole, etaconazole, fenpyrazamine, naftifine, terbinafine, validamycin, pyrimorph, valifenalate, fthalide, probenazole, isotianil, laminarin, extract from Reynoutria sachalinensis, phosphorous acid and salts, teclofthalam, triazoxide, pyriofenone, organic oils, potassium bicarbonate, chlorothalonil, fluoroimide; azoles, including bitertanol, bromuconazole, cyproconazole, difenoconazole, diniconazole, enilconazole, epoxiconazole, fluquinconazole, fenbuconazole, flusilazole, flutriafol, hexaconazole, imibenconazole, ipconazole, metconazole, myclobutanil, penconazole, propiconazole, prothioconazole, simeconazole, triadimefon, triadimenol, tebuconazole, tetraconazole, triticonazole, prochloraz, pefurazoate, imazalil, triflumizole, cyazofamid, benomyl, carbendazim, thia-bendazole, fuberidazole, ethaboxam, etridiazole and hymexazole, azaconazole, diniconazole-M, oxpoconazol, paclobutrazol, uniconazol, 1-(4-chloro-phenyl)-2-([1,2,4]triazol-1-yl)-cycloheptanol and imazalilsulfphate; strobilurins, including azoxystrobin, dimoxystrobin, enestroburin, fluoxastrobin, kresoxim-methyl, metominostrobin, orysastrobin, picoxystrobin, pyraclostrobin, trifloxystrobin, enestroburin, methyl(2-chloro-5-[1-(3-methylbenzyloxyimino)ethyl]benzyl)carbamate, methyl(2-chloro-5-[1-(6-methylpyridin-2-ylmethoxyimino)ethyl]benzyl)carbamate and methyl 2-(ortho-(2,5-dimethylphenyloxymethylene)-phenyl)-3-methoxyacrylate, 2-(2-(6-(3-chloro-2-methyl-phenoxy)-5-fluoro-pyrimidin-4-yloxy)-phenyl)-2-methoxyimino-N-methyl-acetamide and 3-methoxy-2-(2-(N-(4-methoxy-phenyl)-cyclopropanecarboximidoylsulfanylmethyl)-phenyl)-acrylic acid methyl ester; carboxamides, including carboxin, benalaxyl, benalaxyl-M, fenhexamid, flutolanil, furametpyr, mepronil, metalaxyl, mefenoxam, ofurace, oxadixyl, oxycarboxin, penthiopyrad, isopyrazam, thifluzamide, tiadinil, 3,4-dichloro-N-(2-cyanophenyl)isothiazole-5-carboxamide, dimethomorph, flumorph, flumetover, fluopicolide (picobenzamid), zoxamide, carpropamid, diclocymet, mandipropamid, N-(2-(4-[3-(4-chlorophenyl)prop-2-ynyloxy]-3-methoxyphenyl)ethyl)-2-methanesulfonyl-amino-3-methylbutyramide, N-(2-(4-[3-(4-chloro-phenyl)prop-2-ynyloxy]-3-methoxy-phenyl)ethyl)-2-ethanesulfonylamino-3-methylbutyramide, methyl 3-(4-chlorophenyl)-3-(2-isopropoxycarbonyl-amino-3-methyl-butyrylamino)propionate, N-(4′-bromobiphenyl-2-yl)-4-difluoromethyl̂-methylthiazole-δ-carboxamide, N-(4′-trifluoromethyl-biphenyl-2-yl)-4-difluoromethyl-2-methylthiazole-5-carboxamide, N-(4′-chloro-3′-fluorobiphenyl-2-yl)-4-difluoromethyl-2-methyl-thiazole-5-carboxamide, N-(3\4′-dichloro-4-fluorobiphenyl-2-yl)-3-difluoro-methyl-1-methyl-pyrazole-4-carboxamide, N-(3′,4′-dichloro-5-fluorobiphenyl-2-yl)-3-difluoromethyl-1-methylpyrazole-4-carboxamide, N-(2-cyano-phenyl)-3,4-dichloroisothiazole-5-carboxamide, 2-amino-4-methyl-thiazole-5-carboxanilide, 2-chloro-N-(1,1,3-trimethyl-indan-4-yl)-nicotinamide, N-(2-(1,3-dimethylbutyl)-phenyl)-1,3-dimethyl-5-fluoro-1H-pyrazole-4-carboxamide, N-(4′-chloro-3′,5-difluoro-biphenyl-2-yl)-3-difluoromethyl-1-methyl-1H-pyrazole-4-carboxamide, N-(4′-chloro-3′,5-difluoro-biphenyl-2-yl)-3-trifluoromethyl-1-methyl-1H-pyrazole-4-carboxamide, N-(3′,4′-dichloro-5-fluoro-biphenyl-2-yl)-3-trifluoromethyl-1-methyl-1H-pyrazole-4-carboxamide, N-(3′,5-difluoro-4′-methyl-biphenyl-2-yl)-3-difluoromethyl-1-methyl-1H-pyrazole-4-carboxamide, N-(3′,5-difluoro-4′-methyl-biphenyl-2-yl)-3-trifluoromethyl-1-methyl-1H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoro-methyl-1-methyl-1H-pyrazole-4-carboxamide, fluopyram, N-(3-ethyl-3,5-5-trimethyl-cyclohexyl)-3-formylamino-2-hydroxy-benzamide, oxytetracyclin, silthiofam, N-(6-methoxy-pyridin-3-yl)cyclopropanecarboxamide, 2-iodo-N-phenyl-benzamide, N-(2-bicyclo-propyl-2-yl-phenyl)-3-difluormethyl-1-methylpyrazol-4-ylcarboxamide, N-(3′,4′,5′-trifluorobiphenyl-2-yl)-1,3-dimethylpyrazol-4-ylcarboxamide, N-(3′,4′,5′-trifluorobiphenyl-2-yl)-1,3-dimethyl-5-fluoropyrazol-4-yl-carboxamide, N-(3′,4′,5′-trifluorobiphenyl-2-yl)-5-chloro-1,3-dimethyl-pyrazol-4-ylcarboxamide, N-(3′,4′,5′-trifluorobiphenyl-2-yl)-3-fluoromethyl-1-methylpyrazol-4-ylcarboxamide, N-(3′,4′,5′-trifluorobiphenyl-2-yl)-3-(chlorofluoromethyl)-1-methylpyrazol-4-ylcarboxamide, N-(3′,4′,5′-trifluorobiphenyl-2-yl)-3-difluoromethyl-1-methylpyrazol-4-ylcarboxamide, N-(3′,4′,5′-trifluorobiphenyl-2-yl)-3-difluoromethyl-5-fluoro-1-methylpyrazol-4-ylcarboxamide, N-(3′,4′,5′-trifluorobiphenyl-2-yl)-5-chloro-3-difluoromethyl-1-methylpyrazol-4-ylcarboxamide, N-(3′,4′,5′-trifluorobiphenyl-2-yl)-3-(chlorodifluoromethyl)-1-methylpyrazol-4-ylcarboxamide, N-(3′,4′,5′-trifluorobiphenyl-2-yl)-1-methyl-3-trifluoromethylpyrazol-4-ylcarboxamide, N-(3′,4′,5′-trifluorobiphenyl-2-yl)-5-fluoro-1-methyl-3-trifluoromethylpyrazol-4-ylcarboxamide, N-(3′,4′,5′-trifluorobiphenyl-2-yl)-5-chloro-1-methyl-3-trifluoromethylpyrazol-4-ylcarboxamide, N-(2′,4′,5′-trifluorobiphenyl-2-yl)-1,3-dimethylpyrazol-4-ylcarboxamide, N-(2′,4′,5′-trifluorobiphenyl-2-yl)-1,3-dimethyl-5-fluoropyrazol-4-ylcarboxamide, N-(2′,4′,5′-trifluorobiphenyl-2-yl)-5-chloro-1,3-dimethylpyrazol-4-ylcarboxamide, N-(2′,4′,5′-trifluorobiphenyl-2-yl)-3-fluoromethyl-1-methylpyrazol-4-ylcarboxamide, N-(2′,4′,5′-trifluorobiphenyl-2-yl)-3-(chlorofluoromethyl)-1-methylpyrazol-4-ylcarboxamide, N-(2′,4′,5′-trifluorobiphenyl-2-yl)-3-difluoromethyl-1-methylpyrazol-4-ylcarboxamide, N-(2′,4′,5′-trifluorobiphenyl-2-yl)-3-difluoromethyl-5-fluoro-1-methylpyrazol-4-ylcarboxamide, N-(2′,4′,5′-trifluorobiphenyl-2-yl)-5-chloro-3-difluoromethyl-1-methylpyrazol-4-ylcarboxamide, N-(2′,4′,5′-trifluorobiphenyl-2-yl)-3-(chlorodifluoromethyl)-1-methylpyrazol-4-ylcarboxamide, N-(2′,4′,5′-trifluorobiphenyl-2-yl)-1-methyl-3-trifluoromethylpyrazol-4-ylcarboxamide, N-(2′,4′,5′-trifluorobiphenyl-2-yl)-5-fluoro-1-methyl-3-trifluoromethylpyrazol-4-ylcarboxamide, N-(2′,4′,5′-trifluorobiphenyl-2-yl)-5-chloro-1-methyl-3-trifluoromethylpyrazol-4-ylcarboxamide, N-(3′,4′-dichloro-3-fluorobiphenyl-2-yl)-1-methyl-3-trifluoromethyl-1H-pyrazole-4-carboxamide, N-(3′,4′-dichloro-3-fluorobiphenyl-2-yl)-1-methyl-3-difluoromethyl-1H-pyrazole-4-carboxamide, N-(3′,4′-difluoro-3-fluorobiphenyl-2-yl)-1-methyl-3-trifluoromethyl-1H-pyrazole-4-carboxamide, N-(3′,4′-difluoro-3-fluorobiphenyl-2-yl)-1-methyl-S-difluoromethyl-1H-pyrazole-4-carboxamide, N-(3′-chloro-4′-fluoro-3-fluorobiphenyl-2-yl)-1-methyl-3-difluoromethyl-1H-pyrazole-4-carboxamide, N-(3′,4′-dichloro-4-fluorobiphenyl-2-yl)-1-methyl-3-trifluoromethyl-1H-pyrazole-4-carboxamide, N-(3′,4′-difluoro-4-fluorobiphenyl-2-yl)-1-methyl-S-trifluoromethyl-1H-pyrazole-4-carboxamide, N-(3′,4′-dichloro-4-fluorobiphenyl-2-yl)-1-methyl-3-difluoromethyl-1H-pyrazole-4-carboxamide, N-(3′,4′-difluoro-4-fluorobiphenyl-2-yl)-1-methyl-3-difluoromethyl-1H-pyrazole-4-carboxamide, N-(3′-chloro-4′-fluoro-4-fluorobiphenyl-2-yl)-1-methyl-S-difluoromethyl-1H-pyrazole-4-carboxamide, N-(3′,4′-dichloro-5-fluorobiphenyl-2-yl)-1-methyl-3-trifluoromethyl-1H-pyrazole-4-carboxamide, N-(3′,4′-difluoro-5-fluorobiphenyl-2-yl)-1-methyl-3-trifluoromethyl-1H-pyrazole-4-carboxamide, N-(3′,4′-dichloro-5-fluorobiphenyl-2-yl)-1-methyl-S-difluoromethyl-1H-pyrazole-carboxamide, N-(3′,4′-difluoro-5-fluorobiphenyl-2-yl)-1-methyl-3-difluoromethyl-1H-pyrazole-4-carboxamide, N-(3′,4′-dichloro-5-fluorobiphenyl-2-yl)-1,3-dimethyl-1H-pyrazole-4-carboxamide, N-(3′-chloro-4′-fluoro-5-fluorobiphenyl-2-yl)-1-methyl-3-difluoromethyl-1H-pyrazole-4-carboxamide, N-(4′-fluoro-4-fluorobiphenyl-2-yl)-1-methyl-3-trifluoromethyl-1H-pyrazole-4-carboxamide, N-(4′-fluoro-5-fluorobiphenyl-2-yl)-1-methyl-3-trifluoromethyl-1H-pyrazole-4-carboxamide, N-(4′-chloro-5-fluorobiphenyl-2-yl)-1-methyl-3-trifluoromethyl-1H-pyrazole-4-carboxamide, N-(4′-methyl-5-fluorobiphenyl-2-yl)-1-methyl-3-trifluoromethyl-1H-pyrazole-4-carboxamide, N-(4′-fluoro-5-fluorobiphenyl-2-yl)-1,3-dimethyl-1H-pyrazole-4-carboxamide, N-(4′-chloro-5-fluorobiphenyl-2-yl)-1,3-dimethyl-1H-pyrazole-4-carboxamide, N-(4′-methyl-5-fluorobiphenyl-2-yl)-1,3-dimethyl-1H-pyrazole-4-carboxamide, N-(4′-fluoro-6-fluorobiphenyl-2-yl)-1-methyl-3-trifluoromethyl-1H-pyrazole-4-carboxamide, N-(4′-chloro-6-fluorobiphenyl-2-yl)-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[4′-(trifluoromethylthio)-biphenyl-2-yl]-3-difluoromethyl-1-methyl-1H-pyrazole-4-carboxamide and N-[4′-(trifluoromethylthio)-biphenyl-2-yl]-1-methyl-3-trifluoromethyl-1-methyl-1H-pyrazole-4-carboxamide; heterocyclic compounds, including fluazinam, pyrifenox, bupirimate, cyprodinil, fenarimol, ferimzone, mepanipyrim, nuarimol, pyrimethanil, triforine, fenpiclonil, fludioxonil, aldimorph, dodemorph, fenpropimorph, tridemorph, fenpropidin, iprodione, procymidone, vinclozolin, famoxadone, fenamidone, octhilinone, proben-azole, 5-chloro-7-(4-methyl-piperidin-1-yl)-6-(2,4,6-trifluorophenyl)-[1,2,4]triazolo[1,5-a]pyrimidine, anilazine, diclomezine, pyroquilon, proquinazid, tricyclazole, 2-butoxy-6-iodo-3-propylchromen-4-one, acibenzolar-S-methyl, captafol, captan, dazomet, folpet, fenoxanil, quinoxyfen, N,N-dimethyl-3-(3-bromo-6-fluoro-2-methylindole-1-sulfonyl)-[1,2,4]triazole-1-sulfonamide, 5-ethyl-6-octyl-[1,2,4]triazolo[1,5-a]pyrimidin-2,7-diamine, 2,3,5,6-tetrachloro-4-methanesulfonyl-pyridine, 3,4,5-trichloro-pyridine-2,6-di-carbonitrile, N-(1-(5-bromo-3-chloro-pyridin-2-yl)-ethyl)-2,4-dichloro-nicotinamide, N-((5-bromo-3-chloropyridin-2-yl)-methyl)-2,4-dichloro-nicotinamide, diflumetorim, nitrapyrin, dodemorphacetate, fluoroimid, blasticidin-S, chinomethionat, debacarb, difenzoquat, difenzoquat-methylsulphat, oxolinic acid and piperalin; carbamates, including mancozeb, maneb, metam, methasulphocarb, metiram, ferbam, propineb, thiram, zineb, ziram, diethofencarb, iprovalicarb, benthiavalicarb, propamocarb, propamocarb hydrochlorid, 4-fluorophenyl N-(1-(1-(4-cyanophenyl)-ethanesulfonyl)but-2-yl)carbamate, methyl 3-(4-chloro-phenyl)-3-(2-isopropoxycarbonylamino-3-methyl-butyrylamino)propanoate; and other fungicides, including guanidine, dodine, dodine free base, iminoctadine, guazatine, antibiotics: kasugamycin, oxytetracyclin and its salts, streptomycin, polyoxin, validamycin A, nitrophenyl derivatives: binapacryl, dinocap, dinobuton, sulfur-containing heterocyclyl compounds: dithianon, isoprothiolane, organometallic compounds: fentin salts, organophosphorus compounds: edifenphos, iprobenfos, fosetyl, fosetyl-aluminum, phosphorous acid and its salts, pyrazophos, tolclofos-methyl, organochlorine compounds: dichlofluanid, flusulfamide, hexachloro-benzene, phthalide, pencycuron, quintozene, thiophanate, thiophanate-methyl, tolylfluanid, others: cyflufenamid, cymoxanil, dimethirimol, ethirimol, furalaxyl, metrafenone and spiroxamine, guazatine-acetate, iminoc-tadine-triacetate, iminoctadine-tris(albesilate), kasugamycin hydrochloride hydrate, dichlorophen, pentachlorophenol and its salts, N-(4-chloro-2-nitro-phenyl)-N-ethyl-4-methyl-benzenesulfonamide, dicloran, nitrothal-isopropyl, tecnazen, biphenyl, bronopol, diphenylamine, mildiomycin, oxincopper, prohexadione calcium, N-(cyclopropylmethoxyimino-(6-difluoromethoxy-2,3-difluoro-phenyl)-methyl)-2-phenylacetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluormethyl-4-(3-trimethylsilanyl-propoxy)-phenyl)-N-ethyl-N-methylformamidine and N′-(5-difluormethyl-2-methyl-4-(3-trimethylsilanyl-propoxy)-phenyl)-N-ethyl-N-methyl formamidine.

12. The plant seed of claim 10, wherein the fungicide is one or a combination of fluopyram, tebuconazole, chlorothalonil, thiophanate-methyl, prothioconazole, fludioxonil, metalaxyl, or copper hydroxide.

13. The plant seed of claim 10, wherein the insecticide is one or a combination of thiamethoxam, pyrethroids, bifenthrin, tefluthrin, zeta-cypermethrin, organophosphates, chlorethoxyphos, chlorpyrifos, tebupirimphos, cyfluthrin, fiproles, fipronil, nicotinoids, or clothianidin.

14. The plant seed of claim 10, wherein the insecticide comprises bifenthrin.

15. A composition for one or both of benefiting plant growth or conferring protection against pathogenic infection in a susceptible plant, the composition comprising:
a biologically pure culture of Bacillus amyloliquefaciens RTI301 deposited as ATCC No. PTA-121165, or a mutant thereof having all the identifying characteristics thereof, in an amount suitable to benefit plant growth and/or to confer protection against pathogenic infection in the susceptible plant; and
one or a combination of a microbial, a biological, or a chemical insecticide, fungicide, nematicide, bacteriocide, herbicide, plant extract, plant growth regulator, or fertilizer, in an amount suitable to benefit plant growth and/or to confer protection against pathogenic infection in the susceptible plant.

16. The composition of claim 15, wherein the composition is in the form of a liquid and the Bacillus amyloliquefaciens RTI301 is present at a concentration of from about 1.0×10⁸ CFU/ml to about 1.0×10¹² CFU/ml.

17. The composition of claim 15, wherein the composition is in the form of a dust, a dry wettable powder, a spreadable granule, or a dry wettable granule and the Bacillus amyloliquefaciens RTI301 is present in an amount of from about 1.0×10⁸ CFU/g to about 1.0×10¹² CFU/g.

18. The composition of claim 15, wherein the Bacillus amyloliquefaciens RTI301 is in the form of spores or vegetative cells.

19. The composition of claim 15, wherein the fungicide is one or more of benzovindiflupyr, anitiperonosporic, ametoctradin, amisulbrom, copper salts including copper hydroxide, copper oxychloride, copper sulfate, and copper persulfate, boscalid, thiflumazide, flutianil, furalaxyl, thiabendazole, benodanil, mepronil, isofetamid, fenfuram, bixafen, fluxapyroxad, penflufen, sedaxane, coumoxystrobin, enoxastrobin, flufenoxystrobin, pyraoxystrobin, pyrametostrobin, triclopyricarb, fenaminstrobin, metominostrobin, pyribencarb, meptyldinocap, fentin acetate, fentin chloride, fentin hydroxide, oxytetracycline, chlozolinate, chloroneb, tecnazene, etridiazole, iodocarb, prothiocarb, Bacillus subtilis syn., Bacillus amyloliquefaciens including strains QST 713, FZB24, MBI600, and D747, extract from Melaleuca alternifolia, extract from Lupinus albus doce, BLAD polypeptide, pyrisoxazole, oxpoconazole, etaconazole, fenpyrazamine, naftifine, terbinafine, validamycin, pyrimorph, valifenalate, fthalide, probenazole, isotianil, laminarin, extract from Reynoutria sachalinensis, phosphorous acid and salts, teclofthalam, triazoxide, pyriofenone, organic oils, potassium bicarbonate, chlorothalonil, fluoroimide; azoles, including bitertanol, bromuconazole, cyproconazole, difenoconazole, diniconazole, enilconazole, epoxiconazole, fluquinconazole, fenbuconazole, flusilazole, flutriafol, hexaconazole, imibenconazole, ipconazole, metconazole, myclobutanil, penconazole, propiconazole, prothioconazole, simeconazole, triadimefon, triadimenol, tebuconazole, tetraconazole, triticonazole, prochloraz, pefurazoate, imazalil, triflumizole, cyazofamid, benomyl, carbendazim, thia-bendazole, fuberidazole, ethaboxam, etridiazole and hymexazole, azaconazole, diniconazole-M, oxpoconazol, paclobutrazol, uniconazol, 1-(4-chloro-phenyl)-2-([1,2,4]triazol-1-yl)-cycloheptanol and imazalilsulfphate; strobilurins, including azoxystrobin, dimoxystrobin, enestroburin, fluoxastrobin, kresoxim-methyl, metominostrobin, orysastrobin, picoxystrobin, pyraclostrobin, trifloxystrobin, enestroburin, methyl(2-chloro-5-[1-(3-methylbenzyloxyimino)ethyl]benzyl)carbamate, methyl(2-chloro-5-[1-(6-methylpyridin-2-ylmethoxyimino)ethyl]benzyl)carbamate and methyl 2-(ortho-(2,5-dimethylphenyloxymethylene)-phenyl)-3-methoxyacrylate, 2-(2-(6-(3-chloro-2-methyl-phenoxy)-5-fluoro-pyrimidin-4-yloxy)-phenyl)-2-methoxyimino-N-methyl-acetamide and 3-methoxy-2-(2-(N-(4-methoxy-phenyl)-cyclopropanecarboximidoylsulfanylmethyl)-phenyl)-acrylic acid methyl ester; carboxamides, including carboxin, benalaxyl, benalaxyl-M, fenhexamid, flutolanil, furametpyr, mepronil, metalaxyl, mefenoxam, ofurace, oxadixyl, oxycarboxin, penthiopyrad, isopyrazam, thifluzamide, tiadinil, 3,4-dichloro-N-(2-cyanophenyl)isothiazole-5-carboxamide, dimethomorph, flumorph, flumetover, fluopicolide (picobenzamid), zoxamide, carpropamid, diclocymet, mandipropamid, N-(2-(4-[3-(4-chlorophenyl)prop-2-ynyloxy]-3-methoxyphenyl)ethyl)-2-methanesulfonyl-amino-3-methylbutyramide, N-(2-(4-[3-(4-chloro-phenyl)prop-2-ynyloxy]-3-methoxy-phenyl)ethyl)-2-ethanesulfonylamino-3-methylbutyramide, methyl 3-(4-chlorophenyl)-3-(2-isopropoxycarbonyl-amino-3-methyl-butyrylamino)propionate, N-(4′-bromobiphenyl-2-yl)-4-difluoromethyl̂-methylthiazole-δ-carboxamide, N-(4′-trifluoromethyl-biphenyl-2-yl)-4-difluoromethyl-2-methylthiazole-5-carboxamide, N-(4′-chloro-3′-fluorobiphenyl-2-yl)-4-difluoromethyl-2-methyl-thiazole-5-carboxamide, N-(3\4′-dichloro-4-fluorobiphenyl-2-yl)-3-difluoro-methyl-1-methyl-pyrazole-4-carboxamide, N-(3′,4′-dichloro-5-fluorobiphenyl-2-yl)-3-difluoromethyl-1-methylpyrazole-4-carboxamide, N-(2-cyano-phenyl)-3,4-dichloroisothiazole-5-carboxamide, 2-amino-4-methyl-thiazole-5-carboxanilide, 2-chloro-N-(1,1,3-trimethyl-indan-4-yl)-nicotinamide, N-(2-(1,3-dimethylbutyl)-phenyl)-1,3-dimethyl-5-fluoro-1H-pyrazole-4-carboxamide, N-(4′-chloro-3′,5-difluoro-biphenyl-2-yl)-3-difluoromethyl-1-methyl-1H-pyrazole-4-carboxamide, N-(4′-chloro-3′,5-difluoro-biphenyl-2-yl)-3-trifluoromethyl-1-methyl-1H-pyrazole-4-carboxamide, N-(3′,4′-dichloro-5-fluoro-biphenyl-2-yl)-3-trifluoromethyl-1-methyl-1H-pyrazole-4-carboxamide, N-(3′,5-difluoro-4′-methyl-biphenyl-2-yl)-3-difluoromethyl-1-methyl-1H-pyrazole-4-carboxamide, N-(3′,5-difluoro-4′-methyl-biphenyl-2-yl)-3-trifluoromethyl-1-methyl-1H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoro-methyl-1-methyl-1H-pyrazole-4-carboxamide, fluopyram, N-(3-ethyl-3,5-5-trimethyl-cyclohexyl)-3-formylamino-2-hydroxy-benzamide, oxytetracyclin, silthiofam, N-(6-methoxy-pyridin-3-yl) cyclopropanecarboxamide, 2-iodo-N-phenyl-benzamide, N-(2-bicyclo-propyl-2-yl-phenyl)-3-difluormethyl-1-methylpyrazol-4-ylcarboxamide, N-(3′,4′,5′-trifluorobiphenyl-2-yl)-1,3-dimethylpyrazol-4-ylcarboxamide, N-(3′,4′,5′-trifluorobiphenyl-2-yl)-1,3-dimethyl-5-fluoropyrazol-4-yl-carboxamide, N-(3′,4′,5′-trifluorobiphenyl-2-yl)-5-chloro-1,3-dimethyl-pyrazol-4-ylcarboxamide, N-(3′,4′,5′-trifluorobiphenyl-2-yl)-3-fluoromethyl-1-methylpyrazol-4-ylcarboxamide, N-(3′,4′,5′-trifluorobiphenyl-2-yl)-3-(chlorofluoromethyl)-1-methylpyrazol-4-ylcarboxamide, N-(3′,4′,5′-trifluorobiphenyl-2-yl)-3-difluoromethyl-1-methylpyrazol-4-ylcarboxamide, N-(3′,4′,5′-trifluorobiphenyl-2-yl)-3-difluoromethyl-5-fluoro-1-methylpyrazol-4-ylcarboxamide, N-(3′,4′,5′-trifluorobiphenyl-2-yl)-5-chloro-3-difluoromethyl-1-methylpyrazol-4-ylcarboxamide, N-(3′,4′,5′-trifluorobiphenyl-2-yl)-3-(chlorodifluoromethyl)-1-methylpyrazol-4-ylcarboxamide, N-(3′,4′,5′-trifluorobiphenyl-2-yl)-1-methyl-3-trifluoromethylpyrazol-4-ylcarboxamide, N-(3′,4′,5′-trifluorobiphenyl-2-yl)-5-fluoro-1-methyl-3-trifluoromethylpyrazol-4-ylcarboxamide, N-(3′,4′,5′-trifluorobiphenyl-2-yl)-5-chloro-1-methyl-3-trifluoromethylpyrazol-4-ylcarboxamide, N-(2′,4′,5′-trifluorobiphenyl-2-yl)-1,3-dimethylpyrazol-4-ylcarboxamide, N-(2′,4′,5′-trifluorobiphenyl-2-yl)-1,3-dimethyl-5-fluoropyrazol-4-ylcarboxamide, N-(2′,4′,5′-trifluorobiphenyl-2-yl)-5-chloro-1,3-dimethylpyrazol-4-ylcarboxamide, N-(2′,4′,5′-trifluorobiphenyl-2-yl)-3-fluoromethyl-1-methylpyrazol-4-ylcarboxamide, N-(2′,4′,5′-trifluorobiphenyl-2-yl)-3-(chlorofluoromethyl)-1-methylpyrazol-4-ylcarboxamide, N-(2′,4′,5′-trifluorobiphenyl-2-yl)-3-difluoromethyl-1-methylpyrazol-4-ylcarboxamide, N-(2′,4′,5′-trifluorobiphenyl-2-yl)-3-difluoromethyl-5-fluoro-1-methylpyrazol-4-ylcarboxamide, N-(2′,4′,5′-trifluorobiphenyl-2-yl)-5-chloro-3-difluoromethyl-1-methylpyrazol-4-ylcarboxamide, N-(2′,4′,5′-trifluorobiphenyl-2-yl)-3-(chlorodifluoromethyl)-1-methylpyrazol-4-ylcarboxamide, N-(2′,4′,5′-trifluorobiphenyl-2-yl)-1-methyl-3-trifluoromethylpyrazol-4-ylcarboxamide, N-(2′,4′,5′-trifluorobiphenyl-2-yl)-5-fluoro-1-methyl-3-trifluoromethylpyrazol-4-ylcarboxamide, N-(2′,4′,5′-trifluorobiphenyl-2-yl)-5-chloro-1-methyl-3-trifluoromethylpyrazol-4-ylcarboxamide, N-(3′,4′-dichloro-3-fluorobiphenyl-2-yl)-1-methyl-3-trifluoromethyl-1H-pyrazole-4-carboxamide, N-(3′,4′-dichloro-3-fluorobiphenyl-2-yl)-1-methyl-3-difluoromethyl-1H-pyrazole-4-carboxamide, N-(3′,4′-difluoro-3-fluorobiphenyl-2-yl)-1-methyl-3-trifluoromethyl-1H-pyrazole-4-carboxamide, N-(3′,4′-difluoro-3-fluorobiphenyl-2-yl)-1-methyl-S-difluoromethyl-1H-pyrazole-4-carboxamide, N-(3′-chloro-4′-fluoro-3-fluorobiphenyl-2-yl)-1-methyl-3-difluoromethyl-1H-pyrazole-4-carboxamide, N-(3′,4′-dichloro-4-fluorobiphenyl-2-yl)-1-methyl-3-trifluoromethyl-1H-pyrazole-4-carboxamide, N-(3′,4′-difluoro-4-fluorobiphenyl-2-yl)-1-methyl-S-trifluoromethyl-I H-pyrazole-4-carboxamide, N-(3′,4′-dichloro-4-fluorobiphenyl-2-yl)-1-methyl-3-difluoromethyl-1H-pyrazole-4-carboxamide, N-(3′,4′-difluoro-4-fluorobiphenyl-2-yl)-1-methyl-3-difluoromethyl-1H-pyrazole-4-carboxamide, N-(3′-chloro-4′-fluoro-4-fluorobiphenyl-2-yl)-1-methyl-S-difluoromethyl-1H-pyrazole-4-carboxamide, N-(3′,4′-dichloro-5-fluorobiphenyl-2-yl)-1-methyl-3-trifluoromethyl-1H-pyrazole-4-carboxamide, N-(3′,4′-difluoro-5-fluorobiphenyl-2-yl)-1-methyl-3-trifluoromethyl-1H-pyrazole-4-carboxamide, N-(3′,4′-dichloro-5-fluorobiphenyl-2-yl)-1-methyl-S-difluoromethyl-1H-pyrazole-carboxamide, N-(3′,4′-difluoro-5-fluorobiphenyl-2-yl)-1-methyl-3-difluoromethyl-1H-pyrazole-4-carboxamide, N-(3′,4′-dichloro-5-fluorobiphenyl-2-yl)-1,3-dimethyl-1H-pyrazole-4-carboxamide, N-(3′-chloro-4′-fluoro-5-fluorobiphenyl-2-yl)-1-methyl-3-difluoromethyl-1H-pyrazole-4-carboxamide, N-(4′-fluoro-4-fluorobiphenyl-2-yl)-1-methyl-3-trifluoromethyl-1H-pyrazole-4-carboxamide, N-(4′-fluoro-5-fluorobiphenyl-2-yl)-1-methyl-3-trifluoromethyl-1H-pyrazole-4-carboxamide, N-(4′-chloro-5-fluorobiphenyl-2-yl)-1-methyl-3-trifluoromethyl-1H-pyrazole-4-carboxamide, N-(4′-methyl-5-fluorobiphenyl-2-yl)-1-methyl-3-trifluoromethyl-1H-pyrazole-4-carboxamide, N-(4′-fluoro-5-fluorobiphenyl-2-yl)-1,3-dimethyl-1H-pyrazole-4-carboxamide, N-(4′-chloro-5-fluorobiphenyl-2-yl)-1,3-dimethyl-1H-pyrazole-4-carboxamide, N-(4′-methyl-5-fluorobiphenyl-2-yl)-1,3-dimethyl-1H-pyrazole-4-carboxamide, N-(4′-fluoro-6-fluorobiphenyl-2-yl)-1-methyl-3-trifluoromethyl-1H-pyrazole-4-carboxamide, N-(4′-chloro-6-fluorobiphenyl-2-yl)-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[4′-(trifluoromethylthio)-biphenyl-2-yl]-3-difluoromethyl-1-methyl-1H-pyrazole-4-carboxamide and N-[4′-(trifluoromethylthio)-biphenyl-2-yl]-1-methyl-3-trifluoromethyl-1-methyl-1H-pyrazole-4-carboxamide; heterocyclic compounds, including fluazinam, pyrifenox, bupirimate, cyprodinil, fenarimol, ferimzone, mepanipyrim, nuarimol, pyrimethanil, triforine, fenpiclonil, fludioxonil, aldimorph, dodemorph, fenpropimorph, tridemorph, fenpropidin, iprodione, procymidone, vinclozolin, famoxadone, fenamidone, octhilinone, proben-azole, 5-chloro-7-(4-methyl-piperidin-1-yl)-6-(2,4,6-trifluorophenyl)-[1,2,4]triazolo[1,5-a]pyrimidine, anilazine, diclomezine, pyroquilon, proquinazid, tricyclazole, 2-butoxy-6-iodo-3-propylchromen-4-one, acibenzolar-S-methyl, captafol, captan, dazomet, folpet, fenoxanil, quinoxyfen, N,N-dimethyl-3-(3-bromo-6-fluoro-2-methylindole-1-sulfonyl)-[1,2,4]triazole-1-sulfonamide, 5-ethyl-6-octyl-[1,2,4]triazolo[1,5-a]pyrimidin-2,7-diamine, 2,3,5,6-tetrachloro-4-methanesulfonyl-pyridine, 3,4,5-trichloro-pyridine-2,6-di-carbonitrile, N-(1-(5-bromo-3-chloro-pyridin-2-yl)-ethyl)-2,4-dichloro-nicotinamide, N-((5-bromo-3-chloropyridin-2-yl)-methyl)-2,4-dichloro-nicotinamide, diflumetorim, nitrapyrin, dodemorphacetate, fluoroimid, blasticidin-S, chinomethionat, debacarb, difenzoquat, difenzoquat-methylsulphat, oxolinic acid and piperalin; carbamates, including mancozeb, maneb, metam, methasulphocarb, metiram, ferbam, propineb, thiram, zineb, ziram, diethofencarb, iprovalicarb, benthiavalicarb, propamocarb, propamocarb hydrochlorid, 4-fluorophenyl N-(1-(1-(4-cyanophenyl)-ethanesulfonyl)but-2-yl)carbamate, methyl 3-(4-chloro-phenyl)-3-(2-isopropoxycarbonylamino-3-methyl-butyrylamino)propanoate; and other fungicides, including guanidine, dodine, dodine free base, iminoctadine, guazatine, antibiotics: kasugamycin, oxytetracyclin and its salts, streptomycin, polyoxin, validamycin A, nitrophenyl derivatives: binapacryl, dinocap, dinobuton, sulfur-containing heterocyclyl compounds: dithianon, isoprothiolane, organometallic compounds: fentin salts, organophosphorus compounds: edifenphos, iprobenfos, fosetyl, fosetyl-aluminum, phosphorous acid and its salts, pyrazophos, tolclofos-methyl, organochlorine compounds: dichlofluanid, flusulfamide, hexachloro-benzene, phthalide, pencycuron, quintozene, thiophanate, thiophanate-methyl, tolylfluanid, others: cyflufenamid, cymoxanil, dimethirimol, ethirimol, furalaxyl, metrafenone and spiroxamine, guazatine-acetate, iminoc-tadine-triacetate, iminoctadine-tris(albesilate), kasugamycin hydrochloride hydrate, dichlorophen, pentachlorophenol and its salts, N-(4-chloro-2-nitro-phenyl)-N-ethyl-4-methyl-benzenesulfonamide, dicloran, nitrothal-isopropyl, tecnazen, biphenyl, bronopol, diphenylamine, mildiomycin, oxincopper, prohexadione calcium, N-(cyclopropylmethoxyimino-(6-difluoromethoxy-2,3-difluoro-phenyl)-methyl)-2-phenylacetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluormethyl-4-(3-trimethylsilanyl-propoxy)-phenyl)-N-ethyl-N-methylformamidine and N′-(5-difluormethyl-2-methyl-4-(3-trimethylsilanyl-propoxy)-phenyl)-N-ethyl-N-methyl formamidine.

20. The composition of claim 15, wherein the fungicide is one or a combination of fluopyram, tebuconazole, chlorothalonil, thiophanate-methyl, prothioconazole, fludioxonil, metalaxyl, or copper hydroxide.

21. The composition of claim 15, wherein the insecticide is one or a combination of thiamethoxam, pyrethroids, bifenthrin, tefluthrin, zeta-cypermethrin, organophosphates, chlorethoxyphos, chlorpyrifos, tebupirimphos, cyfluthrin, fiproles, fipronil, nicotinoids, or clothianidin.

22. The composition of claim 15, wherein the insecticide comprises bifenthrin and the composition is in a formulation compatible with a liquid fertilizer.

23. A product comprising:
a first composition comprising a biologically pure culture of Bacillus amyloliquefaciens RTI301 deposited as ATCC No. PTA-121165, or a mutant thereof having all the identifying characteristics thereof;
a second composition comprising one or a combination of a microbial, a biological, or a chemical insecticide, fungicide, nematicide, bacteriocide, herbicide, plant extract, plant growth regulator, or fertilizer, wherein the first and second compositions are separately packaged, and wherein each composition is in an amount suitable for one or both of benefiting plant growth or conferring protection against a pathogenic infection in a susceptible plant; and
instructions for delivering in an amount suitable to benefit plant growth, a combination of the first and second compositions to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

24. The product of claim 23, wherein the fungicide is one or more of benzovindiflupyr, anitiperonosporic, ametoctradin, amisulbrom, copper salts including copper hydroxide, copper oxychloride, copper sulfate, and copper persulfate, boscalid, thiflumazide, flutianil, furalaxyl, thiabendazole, benodanil, mepronil, isofetamid, fenfuram, bixafen, fluxapyroxad, penflufen, sedaxane, coumoxystrobin, enoxastrobin, flufenoxystrobin, pyraoxystrobin, pyrametostrobin, triclopyricarb, fenaminstrobin, metominostrobin, pyribencarb, meptyldinocap, fentin acetate, fentin chloride, fentin hydroxide, oxytetracycline, chlozolinate, chloroneb, tecnazene, etridiazole, iodocarb, prothiocarb, Bacillus subtilis syn., Bacillus amyloliquefaciens including strains QST 713, FZB24, MBI600, and D747, extract from Melaleuca alternifolia, extract from Lupinus albus doce, BLAD polypeptide, pyrisoxazole, oxpoconazole, etaconazole, fenpyrazamine, naftifine, terbinafine, validamycin, pyrimorph, valifenalate, fthalide, probenazole, isotianil, laminarin, extract from Reynoutria sachalinensis, phosphorous acid and salts, teclofthalam, triazoxide, pyriofenone, organic oils, potassium bicarbonate, chlorothalonil, fluoroimide; azoles, including bitertanol, bromuconazole, cyproconazole, difenoconazole, diniconazole, enilconazole, epoxiconazole, fluquinconazole, fenbuconazole, flusilazole, flutriafol, hexaconazole, imibenconazole, ipconazole, metconazole, myclobutanil, penconazole, propiconazole, prothioconazole, simeconazole, triadimefon, triadimenol, tebuconazole, tetraconazole, triticonazole, prochloraz, pefurazoate, imazalil, triflumizole, cyazofamid, benomyl, carbendazim, thia-bendazole, fuberidazole, ethaboxam, etridiazole and hymexazole, azaconazole, diniconazole-M, oxpoconazol, paclobutrazol, uniconazol, 1-(4-chloro-phenyl)-2-([1,2,4]triazol-1-yl)-cycloheptanol and imazalilsulfphate; strobilurins, including azoxystrobin, dimoxystrobin, enestroburin, fluoxastrobin, kresoxim-methyl, metominostrobin, orysastrobin, picoxystrobin, pyraclostrobin, trifloxystrobin, enestroburin, methyl (2-chloro-5-[1-(3-methylbenzyloxyimino)ethyl]benzyl)carbamate, methyl(2-chloro-5-[1-(6-methylpyridin-2-ylmethoxyimino)ethyl]benzyl)carbamate and methyl 2-(ortho-(2,5-dimethylphenyloxymethylene)-phenyl)-3-methoxyacrylate, 2-(2-(6-(3-chloro-2-methyl-phenoxy)-5-fluoro-pyrimidin-4-yloxy)-phenyl)-2-methoxyimino-N-methyl-acetamide and 3-methoxy-2-(2-(N-(4-methoxy-phenyl)-cyclopropanecarboximidoylsulfanylmethyl)-phenyl)-acrylic acid methyl ester; carboxamides, including carboxin, benalaxyl, benalaxyl-M, fenhexamid, flutolanil, furametpyr, mepronil, metalaxyl, mefenoxam, ofurace, oxadixyl, oxycarboxin, penthiopyrad, isopyrazam, thifluzamide, tiadinil, 3,4-dichloro-N-(2-cyanophenyl)isothiazole-5-carboxamide, dimethomorph, flumorph, flumetover, fluopicolide (picobenzamid), zoxamide, carpropamid, diclocymet, mandipropamid, N-(2-(4-[3-(4-chlorophenyl)prop-2-ynyloxy]-3-methoxyphenyl)ethyl)-2-methanesulfonyl-amino-3-methylbutyramide, N-(2-(4-[3-(4-chloro-phenyl)prop-2-ynyloxy]-3-methoxy-phenyl)ethyl)-2-ethanesulfonylamino-3-methylbutyramide, methyl 3-(4-chlorophenyl)-3-(2-isopropoxycarbonyl-amino-3-methyl-butyrylamino)propionate, N-(4′-bromobiphenyl-2-yl)-4-difluoromethyl̂-methylthiazole-δ-carboxamide, N-(4′-trifluoromethyl-biphenyl-2-yl)-4-difluoromethyl-2-methylthiazole-5-carboxamide, N-(4′-chloro-3′-fluorobiphenyl-2-yl)-4-difluoromethyl-2-methyl-thiazole-5-carboxamide, N-(3\4′-dichloro-4-fluorobiphenyl-2-yl)-3-difluoro-methyl-1-methyl-pyrazole-4-carboxamide, N-(3′,4′-dichloro-5-fluorobiphenyl-2-yl)-3-difluoromethyl-1-methylpyrazole-4-carboxamide, N-(2-cyano-phenyl)-3,4-dichloroisothiazole-5-carboxamide, 2-amino-4-methyl-thiazole-5-carboxanilide, 2-chloro-N-(1,1,3-trimethyl-indan-4-yl)-nicotinamide, N-(2-(1,3-dimethylbutyl)-phenyl)-1,3-dimethyl-5-fluoro-1H-pyrazole-4-carboxamide, N-(4′-chloro-3′,5-difluoro-biphenyl-2-yl)-3-difluoromethyl-1-methyl-1H-pyrazole-4-carboxamide, N-(4′-chloro-3′,5-difluoro-biphenyl-2-yl)-3-trifluoromethyl-1-methyl-1H-pyrazole-4-carboxamide, N-(3′,4′-dichloro-5-fluoro-biphenyl-2-yl)-3-trifluoromethyl-1-methyl-1H-pyrazole-4-carboxamide, N-(3′,5-difluoro-4′-methyl-biphenyl-2-yl)-3-difluoromethyl-1-methyl-1H-pyrazole-4-carboxamide, N-(3′,5-difluoro-4′-methyl-biphenyl-2-yl)-3-trifluoromethyl-1-methyl-1H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoro-methyl-1-methyl-1H-pyrazole-4-carboxamide, fluopyram, N-(3-ethyl-3,5-5-trimethyl-cyclohexyl)-3-formylamino-2-hydroxy-benzamide, oxytetracyclin, silthiofam, N-(6-methoxy-pyridin-3-yl)cyclopropanecarboxamide, 2-iodo-N-phenyl-benzamide, N-(2-bicyclo-propyl-2-yl-phenyl)-3-difluormethyl-1-methylpyrazol-4-ylcarboxamide, N-(3′,4′,5′-trifluorobiphenyl-2-yl)-1,3-dimethylpyrazol-4-ylcarboxamide, N-(3′,4′,5′-trifluorobiphenyl-2-yl)-1,3-dimethyl-5-fluoropyrazol-4-yl-carboxamide, N-(3′,4′,5′-trifluorobiphenyl-2-yl)-5-chloro-1,3-dimethyl-pyrazol-4-ylcarboxamide, N-(3′,4′,5′-trifluorobiphenyl-2-yl)-3-fluoromethyl-1-methylpyrazol-4-ylcarboxamide, N-(3′,4′,5′-trifluorobiphenyl-2-yl)-3-(chlorofluoromethyl)-1-methylpyrazol-4-ylcarboxamide, N-(3′,4′,5′-trifluorobiphenyl-2-yl)-3-difluoromethyl-1-methylpyrazol-4-ylcarboxamide, N-(3′,4′,5′-trifluorobiphenyl-2-yl)-3-difluoromethyl-5-fluoro-1-methylpyrazol-4-ylcarboxamide, N-(3′,4′,5′-trifluorobiphenyl-2-yl)-5-chloro-3-difluoromethyl-1-methylpyrazol-4-ylcarboxamide, N-(3′,4′,5′-trifluorobiphenyl-2-yl)-3-(chlorodifluoromethyl)-1-methylpyrazol-4-ylcarboxamide, N-(3′,4′,5′-trifluorobiphenyl-2-yl)-1-methyl-3-trifluoromethylpyrazol-4-ylcarboxamide, N-(3′,4′,5′-trifluorobiphenyl-2-yl)-5-fluoro-1-methyl-3-trifluoromethylpyrazol-4-ylcarboxamide, N-(3′,4′,5′-trifluorobiphenyl-2-yl)-5-chloro-1-methyl-3-trifluoromethylpyrazol-4-ylcarboxamide, N-(2′,4′,5′-trifluorobiphenyl-2-yl)-1,3-dimethylpyrazol-4-ylcarboxamide, N-(2′,4′,5′-trifluorobiphenyl-2-yl)-1,3-dimethyl-5-fluoropyrazol-4-ylcarboxamide, N-(2′,4′,5′-trifluorobiphenyl-2-yl)-5-chloro-1,3-dimethylpyrazol-4-ylcarboxamide, N-(2′,4′,5′-trifluorobiphenyl-2-yl)-3-fluoromethyl-1-methylpyrazol-4-ylcarboxamide, N-(2′,4′,5′-trifluorobiphenyl-2-yl)-3-(chlorofluoromethyl)-1-methylpyrazol-4-ylcarboxamide, N-(2′,4′,5′-trifluorobiphenyl-2-yl)-3-difluoromethyl-1-methylpyrazol-4-ylcarboxamide, N-(2′,4′,5′-trifluorobiphenyl-2-yl)-3-difluoromethyl-5-fluoro-1-methylpyrazol-4-ylcarboxamide, N-(2′,4′,5′-trifluorobiphenyl-2-yl)-5-chloro-3-difluoromethyl-1-methylpyrazol-4-ylcarboxamide, N-(2′,4′,5′-trifluorobiphenyl-2-yl)-3-(chlorodifluoromethyl)-1-methylpyrazol-4-ylcarboxamide, N-(2′,4′,5′-trifluorobiphenyl-2-yl)-1-methyl-3-trifluoromethylpyrazol-4-ylcarboxamide, N-(2′,4′,5′-trifluorobiphenyl-2-yl)-5-fluoro-1-methyl-3-trifluoromethylpyrazol-4-ylcarboxamide, N-(2′,4′,5′-trifluorobiphenyl-2-yl)-5-chloro-1-methyl-3-trifluoromethylpyrazol-4-ylcarboxamide, N-(3′,4′-dichloro-3-fluorobiphenyl-2-yl)-1-methyl-3-trifluoromethyl-1H-pyrazole-4-carboxamide, N-(3′,4′-dichloro-3-fluorobiphenyl-2-yl)-1-methyl-3-difluoromethyl-1H-pyrazole-4-carboxamide, N-(3′,4′-difluoro-3-fluorobiphenyl-2-yl)-1-methyl-3-trifluoromethyl-1H-pyrazole-4-carboxamide, N-(3′,4′-difluoro-3-fluorobiphenyl-2-yl)-1-methyl-S-difluoromethyl-1H-pyrazole-4-carboxamide, N-(3′-chloro-4′-fluoro-3-fluorobiphenyl-2-yl)-1-methyl-3-difluoromethyl-1H-pyrazole-4-carboxamide, N-(3′,4′-dichloro-4-fluorobiphenyl-2-yl)-1-methyl-3-trifluoromethyl-1H-pyrazole-4-carboxamide, N-(3′,4′-difluoro-4-fluorobiphenyl-2-yl)-1-methyl-S-trifluoromethyl-1H-pyrazole-4-carboxamide, N-(3′,4′-dichloro-4-fluorobiphenyl-2-yl)-1-methyl-3-difluoromethyl-1H-pyrazole-4-carboxamide, N-(3′,4′-difluoro-4-fluorobiphenyl-2-yl)-1-methyl-3-difluoromethyl-1H-pyrazole-4-carboxamide, N-(3′-chloro-4′-fluoro-4-fluorobiphenyl-2-yl)-1-methyl-S-difluoromethyl-1H-pyrazole-4-carboxamide, N-(3′,4′-dichloro-5-fluorobiphenyl-2-yl)-1-methyl-3-trifluoromethyl-1H-pyrazole-4-carboxamide, N-(3′,4′-difluoro-5-fluorobiphenyl-2-yl)-1-methyl-3-trifluoromethyl-1H-pyrazole-4-carboxamide, N-(3′,4′-dichloro-5-fluorobiphenyl-2-yl)-1-methyl-S-difluoromethyl-1H-pyrazole-carboxamide, N-(3′,4′-difluoro-5-fluorobiphenyl-2-yl)-1-methyl-3-difluoromethyl-1H-pyrazole-4-carboxamide, N-(3′,4′-dichloro-5-fluorobiphenyl-2-yl)-1,3-dimethyl-1H-pyrazole-4-carboxamide, N-(3′-chloro-4′-fluoro-5-fluorobiphenyl-2-yl)-1-methyl-3-difluoromethyl-1H-pyrazole-4-carboxamide, N-(4′-fluoro-4-fluorobiphenyl-2-yl)-1-methyl-3-trifluoromethyl-1H-pyrazole-4-carboxamide, N-(4′-fluoro-5-fluorobiphenyl-2-yl)-1-methyl-3-trifluoromethyl-1H-pyrazole-4-carboxamide, N-(4′-chloro-5-fluorobiphenyl-2-yl)-1-methyl-3-trifluoromethyl-1H-pyrazole-4-carboxamide, N-(4′-methyl-5-fluorobiphenyl-2-yl)-1-methyl-3-trifluoromethyl-1H-pyrazole-4-carboxamide, N-(4′-fluoro-5-fluorobiphenyl-2-yl)-1,3-dimethyl-1H-pyrazole-4-carboxamide, N-(4′-chloro-5-fluorobiphenyl-2-yl)-1,3-dimethyl-1H-pyrazole-4-carboxamide, N-(4′-methyl-5-fluorobiphenyl-2-yl)-1,3-dimethyl-1H-pyrazole-4-carboxamide, N-(4′-fluoro-6-fluorobiphenyl-2-yl)-1-methyl-3-trifluoromethyl-1H-pyrazole-4-carboxamide, N-(4′-chloro-6-fluorobiphenyl-2-yl)-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[4′-(trifluoromethylthio)-biphenyl-2-yl]-3-difluoromethyl-1-methyl-1H-pyrazole-4-carboxamide and N-[4′-(trifluoromethylthio)-biphenyl-2-yl]-1-methyl-3-trifluoromethyl-1-methyl-1H-pyrazole-4-carboxamide; heterocyclic compounds, including fluazinam, pyrifenox, bupirimate, cyprodinil, fenarimol, ferimzone, mepanipyrim, nuarimol, pyrimethanil, triforine, fenpiclonil, fludioxonil, aldimorph, dodemorph, fenpropimorph, tridemorph, fenpropidin, iprodione, procymidone, vinclozolin, famoxadone, fenamidone, octhilinone, proben-azole, 5-chloro-7-(4-methyl-piperidin-1-yl)-6-(2,4,6-trifluorophenyl)-[1,2,4]triazolo[1,5-a]pyrimidine, anilazine, diclomezine, pyroquilon, proquinazid, tricyclazole, 2-butoxy-6-iodo-3-propylchromen-4-one, acibenzolar-S-methyl, captafol, captan, dazomet, folpet, fenoxanil, quinoxyfen, N,N-dimethyl-3-(3-bromo-6-fluoro-2-methylindole-1-sulfonyl)-[1,2,4]triazole-1-sulfonamide, 5-ethyl-6-octyl-[1,2,4]triazolo[1,5-a]pyrimidin-2,7-diamine, 2,3,5,6-tetrachloro-4-methanesulfonyl-pyridine, 3,4,5-trichloro-pyridine-2,6-di-carbonitrile, N-(1-(5-bromo-3-chloro-pyridin-2-yl)-ethyl)-2,4-dichloro-nicotinamide, N-((5-bromo-3-chloropyridin-2-yl)-methyl)-2,4-dichloro-nicotinamide, diflumetorim, nitrapyrin, dodemorphacetate, fluoroimid, blasticidin-S, chinomethionat, debacarb, difenzoquat, difenzoquat-methylsulphat, oxolinic acid and piperalin; carbamates, including mancozeb, maneb, metam, methasulphocarb, metiram, ferbam, propineb, thiram, zineb, ziram, diethofencarb, iprovalicarb, benthiavalicarb, propamocarb, propamocarb hydrochlorid, 4-fluorophenyl N-(1-(1-(4-cyanophenyl)-ethanesulfonyl)but-2-yl)carbamate, methyl 3-(4-chloro-phenyl)-3-(2-isopropoxycarbonylamino-3-methyl-butyrylamino)propanoate; and other fungicides, including guanidine, dodine, dodine free base, iminoctadine, guazatine, antibiotics: kasugamycin, oxytetracyclin and its salts, streptomycin, polyoxin, validamycin A, nitrophenyl derivatives: binapacryl, dinocap, dinobuton, sulfur-containing heterocyclyl compounds: dithianon, isoprothiolane, organometallic compounds: fentin salts, organophosphorus compounds: edifenphos, iprobenfos, fosetyl, fosetyl-aluminum, phosphorous acid and its salts, pyrazophos, tolclofos-methyl, organochlorine compounds: dichlofluanid, flusulfamide, hexachloro-benzene, phthalide, pencycuron, quintozene, thiophanate, thiophanate-methyl, tolylfluanid, others: cyflufenamid, cymoxanil, dimethirimol, ethirimol, furalaxyl, metrafenone and spiroxamine, guazatine-acetate, iminoc-tadine-triacetate, iminoctadine-tris(albesilate), kasugamycin hydrochloride hydrate, dichlorophen, pentachlorophenol and its salts, N-(4-chloro-2-nitro-phenyl)-N-ethyl-4-methyl-benzenesulfonamide, dicloran, nitrothal-isopropyl, tecnazen, biphenyl, bronopol, diphenylamine, mildiomycin, oxincopper, prohexadione calcium, N-(cyclopropylmethoxyimino-(6-difluoromethoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluormethyl-4-(3-trimethylsilanyl-propoxy)-phenyl)-N-ethyl-N-methylformamidine and N′-(5-difluormethyl-2-methyl-4-(3-trimethylsilanyl-propoxy)-phenyl)-N-ethyl-N-methyl formamidine.

25. The product of claim 23, wherein the fungicide is one or a combination of fluopyram, tebuconazole, chlorothalonil, thiophanate-methyl, prothioconazole, fludioxonil, metalaxyl, or copper hydroxide.

26. The product of claim 23, wherein the insecticide is one or a combination of thiamethoxam, pyrethroids, bifenthrin, tefluthrin, zeta-cypermethrin, organophosphates, chlorethoxyphos, chlorpyrifos, tebupirimphos, cyfluthrin, fiproles, fipronil, nicotinoids, or clothianidin.

27. The product of claim 23, wherein the insecticide comprises bifenthrin.

28. The product of claim 23, wherein the first composition further comprises one or a combination of a carrier, a surfactant, a dispersant, or a yeast extract.

29. The product of claim 23, wherein the first composition is in the form of a liquid and the Bacillus amyloliquefaciens RTI301 is present at a concentration of from about 1.0×10⁸ CFU/ml to about 1.0×10¹² CFU/ml.

30. The product of claim 23, wherein the first composition is in the form of a dust, a dry wettable powder, a spreadable granule, or a dry wettable granule and the Bacillus amyloliquefaciens RTI301 is present in an amount of from about 1.0×10⁸ CFU/g to about 1.0×10¹² CFU/g.
